(19) United States
(12) Patent Application Publication
(10) Pub. No.: US 2026/0007774 A1
(43) Pub. Date: Jan. 8, 2026

(54) AAV-MEDIATED DELIVERY OF RGS10 TO MICROGLIA

(71) Applicants: NEURONITY THERAPEUTICS, INC., Mansfield, MA (US); UNIVERSITY OF GEORGIA RESEARCH FOUNDATION, INC., Athens, GA (US)

(72) Inventors: Jae-Kyung LEE, Athens, GA (US); Jaegwon CHUNG, Athens, GA (US); Hyun Joon LEE, Mansfield, MA (US)

(21) Appl. No.: 18/993,682

(22) PCT Filed: Jul. 21, 2023
(86) PCT No.: PCT/US2023/028419

§ 371 Date: Jan. 13, 2025

Related U.S. Application Data
(60) Provisional application No. 63/391,500, filed on Jul. 22, 2022.

(51) Int. Cl. A61K 48/00; C12N 15/86

(52) U.S. Cl. CPC A61K 48/0058; C12N 15/86; C12N 2750/14122; C12N 2750/14143; C12N 2830/008

(57) ABSTRACT

Provided herein are compositions and kits for delivery of RGS10 to microglia via an engineered AAV vector. Also provided here are methods to protect neurons and methods for use of an engineered AAV vector that delivers RGS10 to microglia to treat a disease or disorder in a subject.

### CROSS-REFERENCE

This application claims the benefit of U.S. Provisional Patent Application No. 63/391,500, filed on Jul. 22, 2022, which is incorporated by reference herein in its entirety.

### STATEMENT AS TO FEDERALLY SPONSORED RESEARCH

This invention was made with government support under 1R21NS118224-01 awarded by the National Institute of Neurological Disorders and Stroke (NINDS). The government has certain rights in the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### TECHNICAL FIELD

In one aspect, the present disclosure relates to compositions and kits for delivery of RGS10 via an engineered AAV vector to cells in the central nervous system (CNS) of a subject. The present disclosure also relates to methods for use of the compositions and kits described herein including methods for treatment of a neurodegenerative condition, a metabolic condition, pathological inflammation of the CNS, or a combination thereof in a subject.

### BACKGROUND

Microglia are cells of the brain and spinal cord responsible for immune surveillance and become activated in response to injury, infection, environmental toxins, and other stimuli that threaten neuronal survival. Microglia play a homeostatic role in the CNS and respond to environmental stresses and immunological challenges by scavenging excess neurotoxins and removing dying cells and cellular debris. Animals deficient in Regulator of G-protein Signaling 10 (RGS10), a GTPase activating protein (GAP) for G-protein α subunits develop a parkinsonian phenotype after exposure to chronic systemic inflammation. RGS10 is known to be expressed in both microglia and neurons but functional roles of RGS10 in various cell types is unclear at this time.

### SUMMARY

There remains a need for improved therapies and treatments for conditions including neurodegenerative disorders, metabolic disorders, pathological inflammation of the CNS, and any combination thereof. AAV vector-mediated targeting of RGS10 expression to a subject, including targeted administration of AAV-RGS10 vectors to the CNS provides a solution to the need for improved therapies and treatments for these conditions.

Provided herein are compositions and methods of use thereof for modulating the expression of an endogenous gene, such as increasing RGS10 expression. In some cases, modulating expression of RGS10 mRNA refers to increase in RGS10 mRNA expression. In some cases, modulating expression of RGS10 mRNA refers to a maintenance in RGS10 mRNA expression levels. In some cases, the expression cassette contains a cell-specific promoter sequence. In some cases, the cell-specific promoter sequence drives expression of RGS10 mRNA the CNS. In some cases, the CNS expression of RGS10 mRNA is increased in microglia. In some cases, the CNS expression of RGS10 mRNA is increased in neurons. In some cases, the non-naturally occurring nucleic acid vector refers to a single-stranded DNA vector. In some cases, the single-stranded DNA vector is part of a viral vector. In some cases, the viral vector is an adeno-associated virus (AAV) virus. In some cases, the AAV virus is an AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, or AAV2/AAV12 virus.

Provided herein are methods of enhancing neuron protection. In some aspects, the present disclosure contemplates methods using viral vectors for modulating the expression of an endogenous gene, such as increasing RGS10 expression. In some cases, modulating the expression of an endogenous gene includes modulating expression of an RGS10 mRNA. In some cases, modulating expression of RGS10 mRNA refers to increase in RGS10 mRNA expression. In some cases, modulating expression of RGS10 mRNA refers to a maintenance in RGS10 mRNA expression levels. In some cases, increasing expression of RGS10 mRNA in the CNS enhances neuron protection. In some cases, increasing expression of RGS10 mRNA in microglia, in neurons, or in a combination thereof in the CNS enhances neuron protection. In some cases, increasing expression of RGS10 mRNA in the CNS modulates neuronal activity. In some cases, increasing expression of RGS10 mRNA in the CNS improves cognitive ability. In some cases, the improved cognitive ability will include improved learning, improved memory, or a combination thereof. In some cases, the improved learning or improved memory can be seen as an increase in the extent of long term potentiation in neurons. In some cases, the modulated neuronal activity can be assayed to determine the effect of increasing expression of RGS10 mRNA in the CNS of a subject. In some cases, the modulation of neuronal activity can be assayed by using magnetic resonance imaging (MRI), by measuring field excitatory postsynaptic potential (fEPSP), or by other methods described herein. In some cases, modulating expression of RGS10 mRNA in the CNS to enhance neuron protection, to modulate neuronal activity, or to improve cognitive ability is accomplished in a subject that has a disease or disorder. In some cases, the disease or disorder is neurodegenerative. In some cases, the disease or disorder is a synucleinopathy. In some cases, a subject may also have a metabolic condition. In some cases, the metabolic condition may be obesity, Type 2 diabetes, or another condition described herein. In some cases, a neurodegenerative condition or a metabolic condition may increase inflammation in the CNS. In some cases, a neurodegenerative condition or a metabolic condition may increase oxidative stress in the CNS. In some cases, a neurodegenerative condition or a metabolic condition may disrupt metabolic homeostasis. In some cases, increasing expression of RGS10 mRNA in microglia in a subject may improve or maintain a functional characteristic of the microglia. In some cases, the improved functional characteristic of microglia may include a reduction in excessive response to inflammation. In some cases, the improved functional characteristic of microglia may include an improved response to metabolic stress. In some cases, the improved functional characteristic of microglia may include an improved response to oxidative stress.

Provided herein are methods of treating a disease or disorder including administering an AAV vector which can modulate the amount of RGS10 protein or a functional fragment thereof in the CNS. In some cases, the AAV vector modulates expression of RGS10 mRNA in order to increase the amount of RGS10 protein or a functional fragment thereof in the CNS. In some cases, administering an AAV vector increases the amount of RGS10 protein or a functional fragment thereof in microglia, neurons, or a combination thereof. In some cases, increasing the amount of RGS10 protein or a functional fragment thereof in microglia produces a functional response in the microglia. In some cases, the functional response includes a decreased production or secretion of a proinflammatory cytokine, an increased production or secretion of a neuroprotective substance, or a combination thereof. In some cases, the functional response includes an improvement in an age-related loss of a function. In some cases, the functional response includes an improved response to cellular or environmental stresses. In some cases, the improved response to cellular or environmental stresses includes improved metabolic homeostasis. In some cases, the improved response to cellular or environmental stresses includes improved responses to oxidative stress. In some cases, the improved response to cellular or environmental stresses includes improved responses to inflammation. In some cases, the inflammation is chronic or acute. In some cases, the functional response includes an improvement in microglia uptake of α-synuclein (α-Syn). In some cases, the improvement in microglia uptake of α-Syn includes clearance of extracellular α-Syn aggregates. In some cases, the improvement in microglia uptake of α-Syn includes digestion extracellular α-Syn aggregates after phagocytosis. In some cases, the improved clearance of extracellular α-Syn aggregates decreases an overall amount of α-Syn aggregates in the CNS of a subject. In some cases, the CNS of a subject has an elevated level of α-Syn aggregates compared to the CNS of a reference subject. In some cases, the elevated level of α-Syn aggregates in the subject is reduced following administration of an AAV vector. In some cases, the subject has a disease or disorder. In some cases, the disease or disorder is neurodegenerative. In some cases, the disease or disorder is a synucleinopathy. In some cases, the synucleinopathy is Parkinson's Disease, Dementia with Lewy Body, Essential Tremor, Multiple System Atrophy, Pure Autonomic Failure, or Alzheimer's Disease with Amygdalar Restricted Lewy Bodies (AD/ALB). In some cases, a subject may also have a metabolic condition. In some cases, the metabolic condition may be obesity, Type 2 diabetes, or another condition described herein. In some cases, the subject has not been diagnoses with a disease or disorder. In some cases, the subject is a risk for developing a disease or disorder including a neurodegenerative condition.

Thus, the composition and methods disclosed herein are useful for, among other things, (i) improving the function of microglia in a subject, (ii) restoring the function of microglia in a subject, (iii) decreasing cellular and environmental stresses on cells of the CNS, (iv) reducing the damaging effects of oxidative stress in the CNS, (v) reducing the damaging effects of inflammation in the CNS, (vi) improving a response to pathological metabolic homeostasis in the CNS, (vii) improving cognitive impairment or maintaining cognitive abilities in a subject, (viii), protecting CNS neurons from neurodegeneration, and (ix) treating a neurodegenerative condition.

In one aspect, disclosed herein are methods for increasing a Regulator of G-protein signaling 10 (RGS10) protein level in a brain of a subject in need thereof, the methods comprising administering an adeno-associated virus (AAV) vector comprising a nucleic acid sequence encoding a RGS10 protein or a functional fragment thereof to the subject, wherein the subject has been diagnosed with or is at risk for developing a neurodegenerative disease. In some embodiments, the subject has been diagnosed with a neurodegenerative disease. In some embodiments, the neurodegenerative disease is characterized by an amyloid aggregate pathology. In some embodiments, the amyloid aggregate pathology is α-synuclein pathology, β amyloid peptide (Aβ) pathology, or a combination thereof. In some embodiments, the neurodegenerative disease is Parkinson's Disease, Dementia with Lewy Body, Essential Tremor, Multiple System Atrophy, Pure Autonomic Failure, Alzheimer's Disease, or Alzheimer's Disease with Amygdalar Restricted Lewy Bodies (AD/ALB).

In one aspect, disclosed herein are methods for treating a disease or disorder, the methods comprising administering an adeno-associated virus (AAV) vector comprising a nucleic acid sequence encoding a Regulator of G-protein signaling 10 (RGS10) protein or a functional fragment thereof to a subject in need thereof. In some embodiments, the administering increases expression of RGS10 protein or a functional fragment thereof in a plurality of neurons and a plurality of microglia. In some embodiments, the increased expression of RGS10 protein or a functional fragment thereof decreases an amount of secretion of pro-inflammatory cytokines. In some embodiments, the increased expression of RGS10 protein or a functional fragment thereof increases an amount of secretion of a neuroprotective substance. In some embodiments, the disease or disorder comprises a synucleinopathy. In some embodiments, the synucleinopathy comprises Parkinson's Disease, Dementia with Lewy Body, Essential Tremor, Multiple System Atrophy, Pure Autonomic Failure, Alzheimer's Disease with Amygdalar Restricted Lewy Bodies (AD/ALB), or any combination thereof. In some embodiments, the administering decreases a level of α-syn aggregates in a brain by at least 5%. In some embodiments, the level of α-syn aggregates comprises intracellular α-syn aggregates, extracellular α-syn aggregates, or a combination thereof. In some embodiments, the level of α-syn aggregates is measured quantitatively using an antibody that detects phosphorylated α-syn. In some embodiments, the disease or disorder comprises a metabolic condition. In some embodiments, the subject has Type 2 diabetes, diabetic neuropathy, or obesity, or any combination thereof.

In one aspect, disclosed herein are methods for enhancing neuron protection, the methods comprising administering an adeno-associated virus (AAV) vector comprising a nucleic acid sequence encoding a Regulator of G-protein signaling 10 (RGS10) protein or a functional fragment thereof to a subject in need thereof. In some embodiments, the method further comprises determining a neuron activity in the subject before the administering, after the administering, or before and after the administering. In some embodiments, the neuron activity is determined by measuring long term potentiation (LTP). In some embodiments, the neuron activity is determined by measuring a field excitatory postsynaptic potential (fEPSP) in hippocampal neurons. In some embodiments, the neuron activity is determined through magnetic resonance imaging (MRI). In some embodiments, the subject has a disease or disorder. In some embodiments, the disease or disorder comprises a synucleinopathy. In some embodiments, the disease or disorder further comprises Type 2 diabetes, diabetic neuropathy, or obesity, or any combination thereof. In some embodiments, the synucleinopathy comprises Parkinson's Disease, Dementia with Lewy Body, Essential Tremor, Multiple System Atrophy, Pure Autonomic Failure, or Alzheimer's Disease with Amygdalar Restricted Lewy Bodies (AD/ALB). In some embodiments, a quantitative measurement of neuron activity comprises two or more measurements in the subject at different points in time.

In some aspects, methods for increasing a Regulator of G-protein signaling 10 (RGS10) protein level in a brain of a subject in need thereof, methods for treating a disease or disorder, or methods for enhancing neuron protection may be further contemplated. In some embodiments, a plurality of neurons are protected from a pathological rate of neurodegeneration in the subject. In some embodiments, a plurality of dopaminergic neurons of the midbrain are protected from a pathological rate of neurodegeneration in the subject. In some embodiments, the AAV vector is administered via peripheral injection. In some embodiments, the AAV vector is administered directly to the central nervous system (CNS) of the subject. In some embodiments, the AAV vector is administered directly to the CNS of the subject via intravenous delivery, intravascular delivery, intrathecal delivery, intracisternal delivery, intraspinal delivery, subpial delivery, or intracerebroventricular delivery. In some embodiments, the AAV vector is administered via stereotaxic injection into the brain parenchyma or the spinal cord parenchyma. In some embodiments, the methods comprise delivering the nucleic acid sequence to a plurality of neurons, a plurality of microglia, a plurality of astrocytes or a combination thereof. In some embodiments, the delivering comprises delivering the nucleic acid sequence to a plurality of microglia. In some embodiments, the administering increases a level of RGS10 protein in a plurality of neurons, a plurality of microglia, a plurality of astrocytes, or a combination thereof by at least 5%. In some embodiments, the administering increases a level of RGS10 protein in a plurality of neurons, in a plurality of microglia, or a combination thereof to an extent that compensates for an age-related decrease in brain RGS10 protein levels. In some embodiments, the administering reduces the secretion of proinflammatory cytokines in the plurality of microglia. In some embodiments, the proinflammatory cytokine is tumor necrosis factor alpha (TNFα). In some embodiments, the administering reduces an amount of intracellular reactive oxygen species (ROS) in the plurality of microglia. In some embodiments, the subject demonstrates an improvement in a result from a glucose tolerance test. In some embodiments, the administering increases long term potentiation (LTP) in a plurality of neurons. In some embodiments, the increase in LTP is measured in the hippocampus. In some embodiments, the increase in LTP is measured in a hippocampus. In some embodiments, the increase in LTP is not abrogated by metabolic stress, by inflammatory stress, or by a combination thereof. In some embodiments, the administering results in an improvement of α-syn aggregate pathology in a preformed fibrils (PFFs) model. In some embodiments, the AAV vector is selected from AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, and AAV2/AAV12. In some embodiments, the AAV vector is AAV-9. In some embodiments, the AAV vector is a single stranded AAV (ssAAV). In some embodiments, the ssAAV vector comprises a single chain vector of serotype AAV9. In some embodiments, the AAV vector further comprises an AAV capsid. In some embodiments, the AAV capsid comprises an AAV serotype of AAV-9. In some embodiments, the AAV capsid comprises a pseudotyped AAV capsid. In some embodiments, the AAV vector comprises a cell-specific promoter sequence. In some embodiments, the cell-specific promoter sequence is a pan promoter. In some embodiments, the cell-specific promoter sequence is a pan neuronal or neuron subtype-specific promoter. In some embodiments, the neuron subtype-specific promoter is a dopaminergic neuron-specific promoter. In some embodiments, the cell-specific promoter sequence is a cell-type specific promoter for microglia. In some embodiments, the cell- specific promoter sequence is selected from the group consisting of CamKII, CD68, CMV, F4/80, CX3CR1, CSFR1, Ms4a3, Tmem119, CAG, and IBA1. In some embodiments, the cell-type specific promoter for microglia comprises an IBA1 promoter. In some embodiments, the IBA1 promoter is a human IBA1 promoter. In some embodiments, the IBA1 promoter is a rodent Iba1 promoter. In some embodiments, the IBA1 promoter drives sufficient expression of RGS10 in cells of the CNS. In some embodiments, the IBA1 promoter drives sufficient expression of RGS10 in a plurality of cells of the midbrain. In some embodiments, the IBA1 promoter drives sufficient expression of RGS10 in a plurality of microglia cells of the midbrain. In some embodiments, sufficient expression of RGS10 is maintained for at least 1, 2, 3, 4, 5, 6, 7, 8, 9, 10, 11, or 12 weeks following the administering.

In some embodiments, sufficient expression of RGS10 is maintained for at least 4 weeks following the administering. In some embodiments, the nucleic acid sequence comprises a selected from SEQ ID NO: 1-5. In some embodiments, the nucleic acid sequence comprises a sequence that is at least 80%, 85%, 90%, 95% or 99% identical to SEQ ID NO: 1. In some embodiments, the nucleic acid sequence comprises a sequence that is at least 80%, 85%, 90%, 95% or 99% identical to SEQ ID NO: 2 or SEQ ID NO: 4. In some embodiments, the nucleic acid sequence comprises a sequence that is at least 80%, 85%, 90%, 95% or 99% identical to SEQ ID NO: 3 or SEQ ID NO: 5e. In some embodiments, the nucleic acid sequence comprises a sequence encoding an amino acid sequence selected from SEQ ID NO: 6-8, or a functional fragment thereof. In some embodiments, the nucleic acid sequence comprises a sequence having at least 80%, 81%, 82%, 83%, 84%, 85%, 86%, 87%, 88%, 89%, 90%, 91%, 92, 93%, 94%, 95%, 96%, 97%, 98%, or 99% identity to a sequence encoding an amino acid sequence selected from SEQ ID NO: 6-8, or a functional fragment thereof. In some embodiments, an RGS10 mRNA expression level is elevated in a plurality of neurons, in a plurality of microglia, or in a combination thereof by at least 5% after the administering of the viral vector. In some embodiments, a level of RGS10 protein in the microglia of the subject is increased by at least 5%, 10%, 20%, 30%, 50%, or 100% after the administering of the viral vector. In some embodiments, the administering comprises confining to or concentrating within one or more targeted sites in the CNS of the subject an RGS10 mRNA sequence by administering an AAV6-RGS10 vector to the one or more targeted sites in the CNS of the subject. In some embodiments, the administering comprises spreading an RGS10 mRNA sequence to one or more CNS regions adjacent to one or more targeted sites in the CNS of the subject by administering an AAV9-RGS10 vector to the one or more targeted sites in the CNS of the subject.

In one aspect, disclosed herein are adeno-associated virus (AAV) vectors, the AAV vectors comprising a nucleic acid sequence encoding a Regulator of G-protein signaling 10 (RGS10) protein or a functional fragment thereof. In some embodiments, the AAV vector is selected from AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, and AAV2/AAV12. In some embodiments, the AAV vector is AAV-9. In some embodiments, the AAV vector is a single stranded AAV (ssAAV). In some embodiments, the ssAAV vector comprises a single chain vector of serotype AAV9. In some embodiments, the AAV vector further comprises an AAV capsid. In some embodiments, the AAV capsid comprises an AAV serotype of AAV-9. In some embodiments, the AAV capsid comprises a pseudotyped AAV capsid. In some embodiments, the viral vector comprises a cell-specific promoter sequence. In some embodiments, the cell-specific promoter sequence is a pan neuronal promoter. In some embodiments, the cell-specific promoter sequence is a neuron subtype-specific promoter. In some embodiments, the cell-specific promoter sequence is cell-type specific promoter for microglia. In some embodiments, the cell-specific promoter sequence is selected from the group consisting of CamKII, CD68, CMV, F4/80, CX3CR1, CSFR1, Ms4a3, CAG, and IBA1. In some embodiments, the cell-type specific promoter for microglia comprises an IBA1 promoter. In some embodiments, the IBA1 promoter is a human IBA1 promoter or a rodent Iba1 promoter. In some embodiments, the cell-specific promoter sequence comprises a nucleic acid promoter sequence at least about 90%, 91%, 92%, 93%, 94%, 95%, 96%, 97%, 98%, or 99% identical to a contiguous portion between 15-5522 bp in length of a sequence selected from SEQ ID NO: 9-19. In some embodiments, the cell-specific promoter sequence comprises a nucleic acid promoter sequence identical to a contiguous portion between 15-5522 bp in length of a sequence selected from SEQ ID NO: 9-19. In some embodiments, the cell-specific promoter sequence comprises a nucleic acid promoter sequence at least about 90%, 91%, 92%, 93%, 94%, 95%, 96%, 97%, 98%, or 99% identical to a sequence selected from SEQ ID NO: 9-19. In some embodiments, the cell-specific promoter sequence comprises a nucleic acid promoter sequence identical to a sequence selected from SEQ ID NO: 9-19. In some embodiments, the cell-specific promoter sequence comprises the nucleic acid promoter sequence of SEQ ID NO: 18. In some embodiments, the cell-specific promoter sequence comprises the nucleic acid promoter sequence of SEQ ID NO: 19. In some embodiments, the nucleic acid sequence encoding the RGS10 protein or functional fragment thereof comprises one of SEQ ID NO: 1-5. In some embodiments, the nucleic acid sequence encoding the RGS10 protein or functional fragment thereof comprises a sequence that is at least 80%, 85%, 90%, 95% or 99% identical to SEQ ID NO: 1. In some embodiments, the nucleic acid sequence encoding the RGS10 protein or functional fragment thereof comprises a sequence encoding an amino acid sequence selected from SEQ ID NO: 6-8, or a functional fragment thereof. In some embodiments, the nucleic acid sequence encoding the RGS10 protein or functional fragment thereof comprises a sequence having at least 80%, 81%, 82%, 83%, 84%, 85%, 86%, 87%, 88%, 89%, 90%, 91%, 92, 93%, 94%, 95%, 96%, 97%, 98%, or 99% identity to a sequence encoding an amino acid sequence selected from SEQ ID NO: 6-8, or a functional fragment thereof. In some embodiments, the AAV vector is formulated for direct administration to a central nervous system (CNS) of a subject. In some embodiments, the AAV vector is formulated for direct administration to the microglia of a subject.

In one aspect, disclosed herein are kits comprising i) a composition comprising and AAV vector described herein, and ii) instructions for use. In some embodiments, the kit further comprises a device for delivery of the AAV vector. In some embodiments, the device comprises a catheter or other device for intrathecal administration of the composition. In some embodiments, the device comprises a catheter or other device for intravascular administration of the composition.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph showing expression levels of RGS proteins in primary microglia from mice.

FIG. 2 is a graph showing the effects of RGS10 reduction on TNFα production in a microglia cell line in response to inflammatory and metabolic stresses.

FIG. 3 is a graph showing the effects of RGS10 reduction on TNFα production in a microglia cell line in response to antioxidant treatment.

FIGS. 4A-4B demonstrate impaired α-syn phagocytosis in RGS10 knockdown microglia FIG. 4A is an immunoblot of cell lysates from a microglia cell line demonstrating the effects of RGS10 reduction on levels of α-synuclein (α-syn) phagocytosis. FIG. 4B is a graph of quantitated results from FIG. 4A.

FIGS. 5A-5C demonstrate that loss of RGS10 function leads to weight gain and a reduction in metabolic responsiveness in aged mice. FIG. 5A is a graph and accompanying photograph of weight profile of young and old RGS10 knockout mice demonstrating increased weight gain in aged RGS10 knockout animals. FIG. 5B is a graph of results for a glucose tolerance test in aged RGS10 knockout mice and aged-matched wild-type (WT) controls. FIG. 5C is a graph of results for an insulin sensitivity test in aged RGS10 knockout mice and aged-matched WT controls.

FIGS. 6A-6C demonstrate a role for RGS10 in regulating long term potentiation (LTP) that is impacted by metabolic and inflammatory stress. FIG. 6A shows graphs of field excitatory postsynaptic potentials (fEPSPs) in hippocampal neurons from WT and RGS10 KO mice (male and female graphed separately). FIG. 6B shows graphs of fEPSP slopes versus neuronal stimulation intensity demonstrating reduced hippocampal LTP in RGS10 KO mice compared to WT (male and female graphed separately). FIG. 6C show graphs of the magnitude of normalized hippocampal LTP responses in WT and RGS10 KO mice (male and female graphed separately).

FIGS. 7A-7B show AAV-mediated expression of the green fluorescent protein (GFP) marker in the mouse midbrain following injection of an AAV6-GFP vector. Scale bars across panels indicate 100 μm. FIG. 7A shows GFP fluorescence found mainly in a focal area in the substantia nigra of the midbrain in WT mice 3 weeks following injection of the AAV6-GFP vector demonstrating successful targeting and marker expression using AAV6-based vector. DAPI labeling of nuclei allows for visualization and confirmation of neuroanatomical site of injection in this coronal section of the brain. FIG. 7B shows a higher magnification of the white boxed area in FIG. 7A indicating GFP expression within nuclei of Tyrosine hydroxylase (TH) positive dopaminergic neurons. Panels in FIG. 7B were separated by channel of fluorescence signal. Left panel includes GFP and TH channels as an overlay. Center panel shows channel for GFP only. Right panel shows channel for TH only.

FIG. 8 shows AAV-mediated expression of the GFP marker in the mouse midbrain following injection of an AAV9-GFP vector into the substantia nigra of the midbrain of WT mice. 4 weeks following injection, extensive spreading of GFP is found in the dorsal part of the midbrain. The scale bar across the upper panel indicates 100 μm. TH-positive stained dopaminergic neurons are also visualized in relation to the extent of GFP expression. Panels in FIG. 8 were separated by channel of fluorescence signal. Top panel includes GFP and TH channels as an overlay. Center panel shows channel for GFP only. Bottom panel shows channel for TH only. GFP expression was noted having spread dorsally in the midbrain beyond close proximity to the injection site and beyond labeled TH-positive stained dopaminergic neurons.

FIGS. 9A-9B show AAV9-mediated expression of RGS10 in the mouse midbrain following injection of an AAV9-RGS10 vector into the substantia nigra of the midbrain in RGS10 knockout (KO) mice. Scale bars across panels indicate 100 μm. FIG. 9A shows widespread AAV9-mediated RGS10 expression throughout the midbrain 4 weeks following injection. FIG. 9B shows a high resolution magnification of the white boxed areas in FIG. 9A displaying the expression pattern of RGS10.

### DETAILED DESCRIPTION

### Overview

Synucleinopathies are neurodegenerative diseases that are characterized by abnormal and often excessive accumulation of aggregates of α-Syn protein. These α-Syn aggregates can accumulate in neurons, nerve fibers, glia, and also in the extracellular spaces near these cells. Although many of the underlying biological processes leading to the development of a synucleinopathy in a subject are unknown, the abnormal and excessive accumulation of α-Syn protein aggregates is believed to be a pathological feature that contributes to disease progression and further neurodegeneration. For instance, excessive α-Syn aggregates can be toxic to neurons. However, neurons can also be vulnerable to other insults that can lead to neuronal cell death. Among these, are oxidative stress, inflammation, and the body's response to excessive or extensive inflammation. Exposure, including prolonged exposure, to these various insults can lead to combined or synergistic influences that promote neurodegeneration.

Microglia, the resident macrophages of the CNS, are the first acting and form the main component of active immune defense in the CNS. As such, microglia can be mediators of overall brain maintenance. They can remove infectious agents, remove damaged cells, remove foreign materials, remove nucleic acid fragments, and scavenge the extracellular spaces in the CNS for abnormal proteins or plaques. These removal events can be facilitated by phagocytic actions of microglia followed by digestion of removed material. Promoting inflammation in damaged or infected tissue can be a means by which microglia initiate and facility immune responses. However, overactive microglia can produce unwanted effects. These can include elevated production and secretion of proinflammatory cytokines that serve as extracellular signaling molecules to mediate downstream immune system effects directed toward a given immunogenic insult. These cytokines and the resulting inflammation can produce an added stress on neurons some of which might then undergo apoptosis or necrosis, contributing to a neurodegenerative condition.

Besides removal and clearance of abnormal proteins, cells, and cellular debris, microglia can also help to promote a favorable homeostatic environment in the CNS by other means. One of these is the response of microglia to oxidative stresses. Oxidative stress is known to damage many cell types including neurons. Microglia can produce antioxidant molecules that suppress the formation of free radicals including reactive oxygen species (ROS) and reactive nitrogen species (RNS). Non-limiting examples include glutathione peroxidase, glutathione-s-transferase, phospholipid hydroperoxide glutathione peroxidase (PHGPX), and peroxidase that are known to decompose lipid bydroperoxides to corresponding alcohols. Other non-limiting examples include glutathione peroxidase and catalase which reduce hydrogen peroxide to water. Microglia are also capable of scavenging active free radical species to suppress chain initiation or break the chain propagation reactions that ROS and RNS mediate that have the effect of causing cell damage. Microglia also possess an ability to recognize, degrade and remove oxidatively modified proteins and prevent the accumulation of oxidized proteins, hence serving their ability to repair intracellular damage within their own cells caused by ROS and RNS.

Microglia may provide other means of establishing and supporting a healthy homeostatic environment in the CNS. Microglia can be sensitive in their monitoring of the CNS milieu. Microglia have been found to have direct somatic contacts with neurons and may use these connections in part to potentially monitor neuronal functions. Microglia may be capable of providing neuroprotective effects and secreting neuroprotective molecules as part of homeostatic maintenance of the CNS. Microglia have been shown to engulf synapses which may result in the refinement of synaptic circuitry and may mediate synaptic pruning.

Described herein, in certain embodiments, are compositions and methods for increasing RGS10 levels in the CNS. In some embodiments, compositions and methods described herein may be used to improve, restore, or maintain the function of microglia. In some embodiments, compositions and methods described herein may be used to decrease cellular and environmental stresses on cells of the CNS. In some embodiments, compositions and methods described herein may be used to reduce oxidative stress in the CNS. In some embodiments, compositions and methods described herein may be used to decrease inflammation in the CNS. In some embodiments, compositions and methods described herein may be used to decrease a damaging cellular response to inflammation in the CNS. In some embodiments, compositions and methods described herein may be used to improve a response to pathological metabolic homeostasis in the CNS. In some embodiments, compositions and methods described herein may be used to improve cognitive impairment or maintaining cognitive abilities in a subject. In some embodiments, compositions and methods described herein may be used to protect CNS neurons from neurodegeneration. In some embodiments, compositions and methods described herein may be used to treat a neurodegenerative condition.

### Compositions

Provided herein are compositions for modulating expression of Regulator of G- protein signaling 10 (RGS10). In some embodiments, the composition comprises a viral vector for modulating expression of RGS10. In some embodiments, the viral vector is an AAV. In one aspect, provided herein is an adeno-associated virus (AAV) vector comprising a nucleic acid sequence encoding a RGS10 protein or a functional fragment thereof. In one aspect, provided herein is an adeno-associated virus (AAV) vector comprising a nucleic acid sequence encoding RGS10 mRNA or a functional fragment thereof. In some embodiments described herein, the RGS10 mRNA expressed from the AAV vector is translated in an RGS10 protein or a functional fragment thereof. In some embodiments described herein, the nucleic acid is a DNA. In some embodiments, the nucleic acid is a cDNA. In some embodiments, the protein level of an RGS10 protein or a functional fragment thereof is increased in a plurality of cells receiving the AAV vector. In some embodiments, the plurality of cells receiving the AAV vector comprising target cells. In some embodiments the target cells comprise microglia. In some embodiments, the target cells comprise CNS neurons. In some embodiments, the target cells comprises microglia and CNS neurons. In some embodiments, the nucleic acid vector comprises a cell-specific promoter sequence. In some embodiments, the cell-specific promoter sequence is capable of driving expression of RGS10 mRNA in certain cell types. In some embodiments, the certain cell types comprise microglia. In some embodiments, the certain cell types comprise CNS neurons. In some embodiments, the certain cell types comprise microglia and CNS neurons. In some embodiments, the cell-specific promoter sequence driving expression of RGS10 mRNA in certain cell types modulates a protein level of an RGS10 protein or a functional fragment thereof in the certain cell types. In some embodiments, protein level of an RGS10 protein or a functional fragment thereof in the certain cell types is increased. In some embodiments, the AAV vector is selected from AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, and AAV2/AAV12. In some embodiments, the AAV vector is an AAV9 vector. In some embodiments, the AAV vector is an AAV6 vector.

RGS10 Gene, mRNA, and Protein

RGS10 (regulator of G protein signaling 10) is a gene comprising 6 exons that resides on human Chromosome 10q26.11. The gene encodes RGS10 protein. Three mRNA transcripts encoding RGS10 protein have been predicted. Alternative splicing produces several mRNA transcripts comprising 5 exons. An example of a 181 amino acid sequence derived from a RGS10 mRNA transcript, and further description of RGS10 is included at uniprot.org under accession no. O43665 (last modified Apr. 10, 2019). Regulator of G protein signaling (RGS) family members are regulatory molecules that act as GTPase activating proteins (GAPs) for G alpha subunits of heterotrimeric G proteins. RGS family proteins regulate the activity of small guanine nucleotide-binding (G) proteins to control cellular functions. The activity of RGS proteins can deactivate G protein subunits of the Gi alpha, Go alpha and Gq alpha subtypes. RGS GAPs can drive G proteins into their inactive GDP-bound forms. Regulator of G protein signaling 10 belongs to this family. All RGS proteins share a conserved 120-amino acid sequence termed the RGS domain. RGS10 can regulate G protein-coupled receptor signaling cascades. This regulation can be mediated by inhibition of signal transduction by increasing the GTPase activity of G protein alpha subunits, thereby driving them into their inactive GDP-bound form. RGS10 protein associates specifically with the activated forms of the two related G-protein subunits, G-alphaᵢ₃ and G-alpha_{z} but fails to interact with the structurally and functionally distinct G-alpha subunits. Regulator of G protein signaling 10 protein has been found localized in the nucleus, localized in the cytosol, and associated with the plasma membrane.

In one aspect, provided herein is an AAV vector comprising a nucleic acid sequence encoding RGS10 mRNA or a functional fragment thereof. In some embodiments, the nucleic acid sequence encoding RGS10 mRNA or a functional fragment thereof is transcribed into RGS10 mRNA or a functional fragment thereof. In some embodiments provided herein is an AAV vector comprising a nucleic acid sequence transcribed into RGS10 mRNA or a functional fragment thereof.

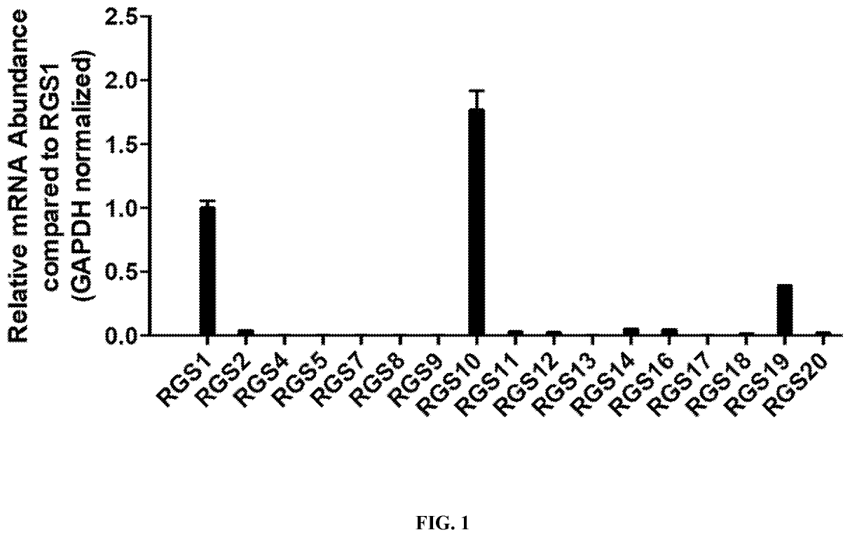

In some embodiments, RGS10 mRNA is endogenously expressed in a plurality of tissue types. In some embodiments, tissue types endogenously expressing RGS10 mRNA comprise adrenal gland, appendix, brain, bone marrow, endometrium, gall bladder, heart, kidney, liver, lung, lymph node, thyroid, spleen, prostate, placenta, ovary, small intestine, stomach, testis, or urinary bladder. In some embodiments, RGS10 mRNA is endogenously expressed in a plurality of cell types. In some embodiments, the cell types endogenously expressing RGS10 mRNA comprise microglia, CNS neurons, macrophages, Hofbauer cells, Langerhans cells, Kupffer cells, monocytes, or granulocytes. In some embodiments, endogenously expressed RGS10 mRNA can be demonstrated to be the most abundant endogenously expressed RGS family member mRNA in microglial cells (FIG. 1).

In some embodiments, the RGS10 mRNA derived from the transcription of an AAV vector comprising a nucleic acid sequence is expressed in a plurality of tissue types. In some embodiments, the plurality of tissue types comprises brain tissue. In some embodiments, the plurality of tissue types comprises neuronal tissue. In some embodiments, the plurality of tissue types comprises glial tissue.

In some embodiments, described herein is an AAV vector comprising a nucleic acid sequence encoding a Regulator of G-protein signaling 10 (RGS10) protein or a functional fragment thereof, wherein the AAV vector is configured such that RGS10 protein is expressed in a plurality of regions of the brain in a subject. In some embodiments, the AAV vector comprising a nucleic acid sequence transcribed into RGS10 mRNA or a functional fragment thereof drives expression of RGS10 mRNA in a plurality of regions of the brain. In some embodiments, the AAV vector is configured such that RGS10 protein is expressed in microglia in a plurality of regions of the brain in a subject. In some embodiments, the plurality of regions of the brain comprise dentate gyrus of the hippocampus, neocortex, cerebellum, dorsal raphe, and dorsal striatum. In some embodiments, the plurality of regions of the brain comprises the forebrain. In some embodiments, the plurality of regions of the brain comprises the midbrain. In some embodiments, the plurality of regions of the brain comprises the forebrain and midbrain. In some embodiments, the plurality of regions of the brain comprises the hindbrain. In some embodiments, the plurality of regions of the brain comprises the spinal cord. In some embodiments, the plurality of regions of the brain comprises the cerebral cortex. In some embodiments, the plurality of regions of the brain comprises the neocortex. In some embodiments, the plurality of regions of the brain comprises the cerebral cortex. In some embodiments, the plurality of regions of the brain comprises the neocortex and hippocampus. In some embodiments, the plurality of regions of the brain comprises a frontal lobe or frontal lobes of the cerebral cortex. In some embodiments, the plurality of regions of the brain comprises a parietal lobe or parietal lobes of the cerebral cortex. In some embodiments, the plurality of regions of the brain comprises a temporal lobe or temporal lobes of the cerebral cortex. In some embodiments, the plurality of regions of the brain comprises an occipital lobe or occipital lobes of the cerebral cortex. In some embodiments, the plurality of regions of the brain comprises a plurality of temporal lobes and a plurality of frontal lobes of the cerebral cortex. In some embodiments, the plurality of regions of the brain comprises the motor cortex. In some embodiments, the plurality of regions of the brain comprises the motor cortex. In some embodiments, the plurality of regions of the brain comprises the primary motor cortex. In some embodiments, the plurality of regions of the brain comprises the supplementary motor cortex. In some embodiments, the plurality of regions of the brain comprises the primary motor cortex. In some embodiments, the plurality of regions of the brain comprises the premotor cortex. In some embodiments, the plurality of regions of the brain comprises the primary motor cortex and the premotor cortex. In some embodiments, the plurality of regions of the brain comprises the primary sensory cortex. In some embodiments, the plurality of regions of the brain comprises the auditory cortex. In some embodiments, the plurality of regions of the brain comprises the posterior parietal cortex. In some embodiments, the plurality of regions of the brain comprises the prefrontal cortex. In some embodiments, the plurality of regions of the brain comprises the ventromedial prefrontal cortex. In some embodiments, the plurality of regions of the brain comprises the lateral prefrontal cortex. In some embodiments, the plurality of regions of the brain comprises the dorsolateral prefrontal cortex. In some embodiments, the plurality of regions of the brain comprises the ventrolateral prefrontal cortex. In some embodiments, the plurality of regions of the brain comprises the medial prefrontal cortex. In some embodiments, the plurality of regions of the brain comprises the ventral prefrontal cortex. In some embodiments, the plurality of regions of the brain comprises the entorhinal cortex. In some embodiments, the plurality of regions of the brain comprises the cingulate cortex. In some embodiments, the plurality of regions of the brain comprises the hippocampus. In some embodiments, the plurality of regions of the brain comprises the dentate gyrus of the hippocampus. In some embodiments, the plurality of regions of the brain comprises the hippocampus. In some embodiments, the plurality of regions of the brain comprises the amygdala. In some embodiments, the plurality of regions of the brain comprises the insula. In some embodiments, the plurality of regions of the brain comprises the basal ganglia. In some embodiments, the plurality of regions of the brain comprises the striatum. In some embodiments, the plurality of regions of the brain comprises the pallidum. In some embodiments, the plurality of regions of the brain comprises the dorsal striatum. In some embodiments, the plurality of regions of the brain comprises the posterior dorsomedial striatum (pDMS). In some embodiments, the plurality of regions of the brain comprises the caudate. In some embodiments, the plurality of regions of the brain comprises the putamen. In some embodiments, the plurality of regions of the brain comprises the substantia nigra. In some embodiments, the plurality of regions of the brain comprises the substantia nigra pars compacta. In some embodiments, the plurality of regions of the brain comprises the substantia nigra pars reticulata. In some embodiments, the plurality of regions of the brain comprises the globus pallidus. In some embodiments, the plurality of regions of the brain comprises the thalamus. In some embodiments, the plurality of regions of the brain comprises the globus pallidus. In some embodiments, the plurality of regions of the brain comprises the subthalamic nucleus. In some embodiments, the plurality of regions of the brain comprises the ventral tegmentum. In some embodiments, the plurality of regions of the brain comprises the cerebellum. In some embodiments, the AAV vector comprising a nucleic acid sequence transcribed into RGS10 mRNA or a functional fragment thereof drives expression of RGS10 mRNA in regions of the brain in which RGS10 mRNA is endogenously expressed. In some embodiments, the AAV vector comprising a nucleic acid sequence transcribed into RGS10 mRNA or a functional fragment thereof is configured to be administered to one or more sites in the CNS and expression of the sequence transcribed into RGS10 mRNA or a functional fragment thereof is intended to spread to a plurality of adjacent regions of the brain in the subject. In some embodiments, an AAV9 vector is configured to be administered to one or more sites in the CNS and expression of the sequence transcribed into RGS10 mRNA or a functional fragment thereof is intended to spread to a plurality of adjacent regions of the brain in the subject.

In some embodiments, described herein is an AAV vector comprising a nucleic acid sequence encoding a Regulator of G-protein signaling 10 (RGS10) protein or a functional fragment thereof, wherein the AAV vector is configured such that RGS10 protein is expressed in a single region of the brain in a subject. In some embodiments, the AAV vector comprising a nucleic acid sequence transcribed into RGS10 mRNA or a functional fragment thereof drives expression of RGS10 mRNA in a single region of the brain. In some embodiments, the AAV vector is configured such that RGS10 protein is expressed in microglia in a subject in a single region of the brain. In some embodiments, the single region of the brain is selected from dentate gyrus of the hippocampus, neocortex, cerebellum, dorsal raphe, and dorsal striatum. In some embodiments, the single region of the brain comprises the forebrain. In some embodiments, the single region of the brain comprises the midbrain. In some embodiments, the single region of the brain comprises the forebrain and midbrain. In some embodiments, the single region of the brain comprises the hindbrain. In some embodiments, the single region of the brain comprises the spinal cord. In some embodiments, the single region of the brain comprises the cerebral cortex. In some embodiments, the single region of the brain comprises the neocortex. In some embodiments, the single region of the brain comprises the cerebral cortex. In some embodiments, the single region of the brain comprises the neocortex and hippocampus. In some embodiments, the single region of the brain comprises a frontal lobe or both frontal lobes of the cerebral cortex. In some embodiments, the single region of the brain comprises a parietal lobe or both parietal lobes of the cerebral cortex. In some embodiments, the single region of the brain comprises a temporal lobe or both temporal lobes of the cerebral cortex. In some embodiments, the single region of the brain comprises an occipital lobe or both occipital lobes of the cerebral cortex. In some embodiments, the single region of the brain comprises the motor cortex. In some embodiments, the single region of the brain comprises the primary motor cortex. In some embodiments, the single region of the brain comprises the supplementary motor cortex. In some embodiments, the single region of the brain comprises the premotor cortex. In some embodiments, the single region of the brain comprises the primary motor cortex and the premotor cortex. In some embodiments, the single region of the brain comprises the primary sensory cortex. In some embodiments, the single region of the brain comprises the auditory cortex. In some embodiments, the single region of the brain comprises the posterior parietal cortex. In some embodiments, the single region of the brain comprises the prefrontal cortex. In some embodiments, the single region of the brain comprises the ventromedial prefrontal cortex. In some embodiments, the single region of the brain comprises the lateral prefrontal cortex. In some embodiments, the single region of the brain comprises the dorsolateral prefrontal cortex. In some embodiments, the single region of the brain comprises the ventrolateral prefrontal cortex. In some embodiments, the single region of the brain comprises the medial prefrontal cortex. In some embodiments, the single region of the brain comprises the ventral prefrontal cortex. In some embodiments, the single region of the brain comprises the entorhinal cortex. In some embodiments, the single region of the brain comprises the cingulate cortex. In some embodiments, the single region of the brain comprises the hippocampus. In some embodiments, the single region of the brain comprises the dentate gyrus of the hippocampus. In some embodiments, the single region of the brain comprises the amygdala. In some embodiments, the single region of the brain comprises the insula. In some embodiments, the single region of the brain comprises the basal ganglia. In some embodiments, the single region of the brain comprises the striatum. In some embodiments, the single region of the brain comprises the pallidum. In some embodiments, the single region of the brain comprises the dorsal striatum. In some embodiments, the single region of the brain comprises the posterior dorsomedial striatum (pDMS). In some embodiments, the single region of the brain comprises the caudate. In some embodiments, the single region of the brain comprises the putamen. In some embodiments, the single region of the brain comprises the substantia nigra. In some embodiments, the single region of the brain comprises the substantia nigra pars compacta. In some embodiments, the single region of the brain comprises the substantia nigra pars reticulata. In some embodiments, the single region of the brain comprises the globus pallidus. In some embodiments, the single region of the brain comprises the thalamus. In some embodiments, the single region of the brain comprises the globus pallidus. In some embodiments, the single region of the brain comprises the subthalamic nucleus. In some embodiments, the single region of the brain comprises the ventral tegmentum. In some embodiments, the single region of the brain comprises the cerebellum. In some embodiments, the AAV vector comprising a nucleic acid sequence transcribed into RGS10 mRNA or a functional fragment thereof drives expression of RGS10 mRNA in a single region of the brain in which RGS10 mRNA is endogenously expressed. In some embodiments, the AAV vector comprising a nucleic acid sequence transcribed into RGS10 mRNA or a functional fragment thereof is configured to be administered to one or more sites in the CNS and expression of the sequence transcribed into RGS10 mRNA or a functional fragment thereof is intended to remain confined to or concentrated within a single region of the brain in the subject. In some embodiments, an AAV6 vector is configured to be administered to one or more sites in the CNS and expression of the sequence transcribed into RGS10 mRNA or a functional fragment thereof is intended to remain confined to or concentrated within a single region of the brain in the subject.

In some embodiments, described herein is an AAV vector comprising a nucleic acid sequence encoding a Regulator of G-protein signaling 10 (RGS10) protein or a functional fragment thereof, wherein the AAV vector is configured such that the RGS10 protein is expressed in a plurality of cell types of the brain in a subject. In some embodiments, the AAV vector comprising a nucleic acid sequence transcribed into RGS10 mRNA or a functional fragment thereof drives expression of RGS10 mRNA in a plurality of cell types of the brain. In some embodiments, the plurality of cell types of the brain comprises glial cells. In some embodiments, the plurality of cell types of the brain comprises microglia. In some embodiments, the plurality of cell types of the brain comprises astrocytes. In some embodiments, the plurality of cell types of the brain comprise oligodendrocytes. In some embodiments, the plurality of cell types of the brain comprises ependymal cells. In some embodiments, the plurality of cell types of the brain comprises neurons. In some embodiments, the plurality of cell types of the brain comprises dopaminergic neurons. In some embodiments, the plurality of cell types of the brain comprises GABAergic neurons. In some embodiments, the plurality of cell types of the brain comprises dopaminergic neurons and GABAergic neurons. In some embodiments, the plurality of cell types of the brain comprises glutamatergic neurons. In some embodiments, the plurality of cell types of the brain comprises upper motor neurons. In some embodiments, the plurality of cell types of the brain comprises projection neurons. In some embodiments, the plurality of cell types of the brain comprises interneurons. In some embodiments, the plurality of cell types of the brain comprises pyramidal cells. In some embodiments, the plurality of cell types of the brain comprises Purkinje cells. In some embodiments, the plurality of cell types of the brain comprises granule cells. In some embodiments, the plurality of cell types of the brain comprises stellate cells. In some embodiments, the plurality of cell types of the brain comprises fusiform cells. In some embodiments, the plurality of cell types of the brain comprises basket cells. In some embodiments, the plurality of cell types of the brain comprises dentate gyrus granule cells. In some embodiments, the plurality of cell types of the brain comprises cortical projection neurons. In some embodiments, the plurality of cell types of the brain comprises dentate gyrus granule cells and cortical projection neurons. In some embodiments, the plurality of cell types of the brain comprises cortical projection neurons. In some embodiments, the plurality of cell types of the brain comprises forebrain interneurons. In some embodiments, the plurality of cell types of the brain comprises striatal projection neurons. In some embodiments, the plurality of cell types of the brain comprises indirect spiny projection neurons (iSPNs) in the pDMS. In some embodiments, the iSPNs project to the substantia nigra pars reticulata (SNr) indirectly via the external segment of the globus pallidus (GPe) and subthalamic nucleus. In some embodiments, the iSPNs prominently express dopamine D2 receptor. In some embodiments, the AAV vector comprising a nucleic acid sequence transcribed into RGS10 mRNA or a functional fragment thereof drives expression of RGS10 mRNA in cell types in which RGS10 mRNA is endogenously expressed.

In some embodiments, described herein is an AAV vector comprising a nucleic acid sequence encoding a Regulator of G-protein signaling 10 (RGS10) protein or a functional fragment thereof, wherein the AAV vector is configured such that the RGS10 protein is expressed in a single cell type of the brain in a subject. In some embodiments, the AAV vector comprising a nucleic acid sequence transcribed into RGS10 mRNA or a functional fragment thereof drives expression of RGS10 mRNA in a single cell type of the brain. In some embodiments, the single cell type of the brain comprises glial cells. In some embodiments, the single cell type of the brain comprises microglia. In some embodiments, the single cell type of the brain comprises astrocytes. In some embodiments, the single cell type of the brain comprise oligodendrocytes. In some embodiments, the single cell type of the brain comprises ependymal cells. In some embodiments, the single cell type of the brain comprises neurons. In some embodiments, the single cell type of the brain comprises dopaminergic neurons. In some embodiments, the single cell type of the brain comprises GABAergic neurons. In some embodiments, the single cell type of the brain comprises glutamatergic neurons. In some embodiments, the single cell type of the brain comprises upper motor neurons. In some embodiments, the single cell type of the brain comprises projection neurons. In some embodiments, the single cell type of the brain comprises interneurons. In some embodiments, the single cell type of the brain comprises pyramidal cells. In some embodiments, the single cell type of the brain comprises Purkinje cells. In some embodiments, the single cell type of the brain comprises granule cells. In some embodiments, the single cell type of the brain comprises stellate cells. In some embodiments, the single cell type of the brain comprises fusiform cells. In some embodiments, the single cell type of the brain comprises basket cells. In some embodiments, the single cell type of the brain comprises dentate gyrus granule cells. In some embodiments, the single cell type of the brain comprises cortical projection neurons. In some embodiments, the single cell type of the brain comprises cortical projection neurons. In some embodiments, the single cell type of the brain comprises forebrain interneurons. In some embodiments, the single cell type of the brain comprises striatal projection neurons. In some embodiments, the single cell type of the brain comprises indirect spiny projection neurons (iSPNs) in the pDMS. In some embodiments, the iSPNs project to the substantia nigra pars reticulata (SNr) indirectly via the external segment of the globus pallidus (GPe) and subthalamic nucleus. In some embodiments, the iSPNs prominently express dopamine D2 receptor. In some embodiments, the AAV vector comprising a nucleic acid sequence transcribed into RGS10 mRNA or a functional fragment thereof drives expression of RGS10 mRNA in a single cell type in which RGS10 mRNA is endogenously expressed.

In some embodiments, provided herein is an AAV vector comprising a nucleic acid sequence encoding an RGS10 protein. In some embodiments, the nucleic acid sequence comprises a sequence selected from AF368902, AF045229, AF493934, AK290773, CR457008, and BC009361, see Uniprot entry 043665. In some embodiments, the nucleic acid sequence comprises a sequence having at least 80%, 85%, 90%, 95%, or 99% identity to any one of AF368902, AF045229, AF493934, AK290773, CR457008, and BC009361. In some embodiments, the nucleic acid sequence comprises a sequence selected from SEQ ID NO: 1-5. In some embodiments, the nucleic acid sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1-5. In some embodiments, the nucleic acid sequence comprises a functional fragment of any sequence of SEQ ID NO: 1-5. In some embodiments, the nucleic acid sequence comprises SEQ ID NO: 1. In some embodiments, the nucleic acid sequence comprises SEQ ID NO: 2. In some embodiments, the nucleic acid sequence comprises SEQ ID NO: 3. In some embodiments, the nucleic acid sequence comprises SEQ ID NO: 4. In some embodiments, the nucleic acid sequence comprises SEQ ID NO: 5. Table 1 lists exemplary nucleic acid sequences that can be used in an AAV vector described herein. The nucleic acid sequences listed in Table 1 are RGS10cDNA sequences.

TABLE 1
RGS10 cDNA sequences

| SEQ ID NO:/ | |
|---|---|
| Sequence Name | Nucleic acid sequence |
| SEQ ID NO: 1 / | ATGCAGTCTGAACTTTGCTTTGCAGACATCCACGACAGCGATGGCAGTTCCAGCA |
| AF368902.1 | GCAGCCACCAGAGCCTCAAGAGCACAGCCAAATGGGCGGCATCCCTGGAGAATCT |
| | GCTGGAAGACCCAGAAGGCGTGAAAAGATTTAGGGAATTTTTAAAAAAGGAATTC |
| | AGTGAAGAAAATGTTTTGTTTTGGCTAGCATGTGAAGATTTTAAGAAAATGCAAG |
| | ATAAGACGCAGATGCAGGAAAAGGCAAAGGAGATCTACATGACCTTTCTGTCCAG |
| | CAAGGCCTCATCACAGGTCAACGTGGAGGGGCAGTCTCGGCTCAACGAGAAGATC |
| | CTGGAAGAACCGCACCCTCTGATGTTCCAGAAACTCCAGGACCAGATCTTTAATC |
| | TCATGAAGTACGACAGCTACAGCCGCTTCTTAAAGTCTGACTTGTTTTTAAAACA |
| | CAAGCGAACCGAGGAAGAGGAAGAAGATTTGCCTGATGCTCAAACTGCAGCTAAA |
| | AGAGCTTCCAGAATTTATAACACATGA |
| SEQ ID NO: 2/ | TTTCTTCCTCCTCGGGCGCCCGCGGCGATGTTCAACCGCGCCGTGAGCCGGCTGA |
| AK290773.1 | GCAGGAAGCGGCCGCCGTCAGACATCCACGACAGCGATGGCAGTTCCAGCAGCAG |
| | CCACCAGAGCCTCAAGAGCACAGCCAAATGGGCGGCATCCCTGGAGAATCTGCTG |
| | GAAGACCCAGAAGGCGTGAAAAGATTTAGGGAATTTTTAAAAAAGGAATTCAGTG |
| | AAGAAAATGTTTTGTTTTGGCTAGCATGTGAAGATTTTAAGAAAATGCAAGATAA |
| | GACGCAGATGCAGGAAAAGGCAAAGGAGATCTACATGACCTTTCTGTCCAGCAAG |
| | GCCTCATCACAGGTCAACGTGGAGGGGCAGTCTCGGCTCAACGAGAAGATCCTGG |
| | AAGAACCGCACCCTCTGATGTTCCAGAAACTCCAGGACCAGATCTTTAATCTCAT |
| | GAAGTACGACAGCTACAGCCGCTTCTTAAAGTCTGACTTGTTTTTAAAACACAAG |
| | CGAACCGAGGAAGAGGAAGAAGATTTGCCTGATGCTCAAACTGCAGCTAAAAGAG |
| | CTTCCAGAATTTATAACACATGAGCCCCCAAAAAGCCGGGACTGGCAGCTTTAAG |
| | AAGCAAAGGAATTTCCTCTCAGGACCGTGCCGGGTTTATCATTGCTTTGTTATTT |
| | GTAAGGACTGAAATGTACAAAACCCTTCAATGGGATGTGTGTTTTATTAACTGCT |
| | TCACCAGTAAATTTTGCATGATGGCTAAGCTAA |
| SEQ ID NO: 3/ | GGATTGTTGGTCTGCGTGGAACTTCTCAGGTGGACACCAGAGCATGGAACACATC |
| AF045229.2 | CACGACAGCGATGGCAGTTCCAGCAGCAGCCACCAGAGCCTCAAGAGCACAGCCA |
| | AATGGGCGGCATCCCTGGAGAATCTGCTGGAAGACCCAGAAGGCGTGAAAAGATT |
| | TAGGGAATTTTTAAAAAAGGAATTCAGTGAAGAAAATGTTTTGTTTTGGCTAGCA |
| | TGTGAAGATTTTAAGAAAATGCAAGATAAGACGCAGATGCAGGAAAAGGCAAAGG |
| | AGATCTACATGACCTTTCTGTCCAGCAAGGCCTCATCACAGGTCAACGTGGAGGG |
| | GCAGTCTCGGCTCAACGAGAAGATCCTGGAAGAACCGCACCCTCTGATGTTCCAG |
| | AAACTCCAGGACCAGATCTTTAATCTCATGAAGTACGACAGCTACAGCCGCTTTC |
| | TTAAGTCTGACTTGTTTTTAAAACACAAGCGAACCGAGGAAGAGGAAGAAGATTT |
| | GCCTGATGCTCAAACTGCAGCTAAAAGAGCTTCCAGAATTTATAACACATGAGCC |
| | CCCAAAAAGCCGGGACTGGCAGCTTTAAGAAGCAAAGGAATTTCCTCTCAGGACG |
| | TGCCGGGTTTATCATTGCTTTGTTATTTGTAAGGACTGAAATGTACAAAACCCTT |
| | CAAT |
| SEQ ID NO: 4/ | TTTCTTCCTCCTCGGGCGCCCGCGGCGATGTTCAACCGCGCCGTGAGCCGGCTGA |
| RGS10 cDNA | GCAGGAAGCGGCCGCCGTCAGACATCCACGACAGCGATGGCAGTTCCAGCAGCAG |
| v4-(resides | CCACCAGAGCCTCAAGAGCACAGCCAAATGGGCGGCATCCCTGGAGAATCTGCTG |
| within | GAAGACCCAGAAGGCGTGAAAAGATTTAGGGAATTTTTAAAAAAGGAATTCAGTG |
| AK290773.1) | AAGAAAATGTTTTGTTTTGGCTAGCATGTGAAGATTTTAAGAAAATGCAAGATAA |
| | GACGCAGATGCAGGAAAAGGCAAAGGAGATCTACATGACCTTTCTGTCCAGCAAG |
| | GCCTCATCACAGGTCAACGTGGAGGGGCAGTCTCGGCTCAACGAGAAGATCCTGG |
| | AAGAACCGCACCCTCTGATGTTCCAGAAACTCCAGGACCAGATCTTTAATCTCAT |
| | GAAGTACGACAGCTACAGCCGCTTCTTAAAGTCTGACTTGTTTTTAAAACACAAG |
| | CGAACCGAGGAAGAGGAAGAAGATTTGCCTGATGCTCAAACTGCAGCTAAAAGAG |
| | CTTCCAGAATTTATAACACATGA |
| SEQ ID NO: 5/ | GGATTGTTGGTCTGCGTGGAACTTCTCAGGTGGACACCAGAGCATGGAACACATC |
| RGS10 cDNA | CACGACAGCGATGGCAGTTCCAGCAGCAGCCACCAGAGCCTCAAGAGCACAGCCA |
| v5-(resides | AATGGGCGGCATCCCTGGAGAATCTGCTGGAAGACCCAGAAGGCGTGAAAAGATT |
| within | TAGGGAATTTTTAAAAAAGGAATTCAGTGAAGAAAATGTTTTGTTTTGGCTAGCA |
| AF045229.2) | TGTGAAGATTTTAAGAAAATGCAAGATAAGACGCAGATGCAGGAAAAGGCAAAGG |
| | AGATCTACATGACCTTTCTGTCCAGCAAGGCCTCATCACAGGTCAACGTGGAGGG |
| | GCAGTCTCGGCTCAACGAGAAGATCCTGGAAGAACCGCACCCTCTGATGTTCCAG |
| | AAACTCCAGGACCAGATCTTTAATCTCATGAAGTACGACAGCTACAGCCGCTTTC |
| | TTAAGTCTGACTTGTTTTTAAAACACAAGCGAACCGAGGAAGAGGAAGAAGATTT |
| | GCCTGATGCTCAAACTGCAGCTAAAAGAGCTTCCAGAATTTATAACACATGA |

In some embodiments of an AAV vector provided herein a nucleic acid sequence encoding an RGS10 protein comprises a sequence encoding an amino acid sequence selected from SEQ ID NO: 6-8, or a functional fragment thereof. In some embodiments, the nucleic acid sequence comprises a sequence having at least 80%, 81%, 82%, 83%, 84%, 85%, 86%, 87%, 88%, 89%, 90%, 91%, 92, 93%, 94%, 95%, 96%, 97%, 98%, or 99% identity to a sequence encoding an amino acid sequence selected from SEQ ID NO: 6-8, or a functional fragment thereof. In some embodiments, the nucleic acid sequence comprises a sequence encoding the amino acid sequence selected from AAK52979.1, AAC03783.1, AAM12648.1, BAF83462.1, CAG33289.1, EAW49389.1, EAW49390.1, and AAH09361.1 (see Uniprot entry 043665). In some embodiments, the nucleic acid sequence comprises a sequence encoding the amino acid sequence selected from AAK52979.1, AAC03783.1, AAM12648.1, BAF83462.1, CAG33289.1, EAW49389.1, EAW49390.1, and AAH09361.1, or a functional fragment thereof. In some embodiments, the nucleic acid sequence comprises a sequence encoding the amino acid sequence SEQ ID NO: 6. In some embodiments, the nucleic acid sequence comprises a sequence encoding the amino acid sequence SEQ ID NO: 7. In some embodiments, the nucleic acid sequence comprises a sequence encoding the amino acid sequence SEQ ID NO: 8. Table 2 lists exemplary RGS10 protein sequences in which an AAV vector comprising a nucleic acid sequence described herein encodes. The RGS10 protein amino acid sequences listed in Table 2 can be encoded by RGS10 cDNA sequences.

TABLE 2
RGS10 protein amino acid sequences

| SEQ ID NO:/ | |
|---|---|
| (Sequence Name) | Amino acid sequence |
| SEQ ID NO: 6/ | MQSELCFADIHDSDGSSSSSHQSLKSTAKWAASLENLLEDPEGVKRFREFL |
| (AAK52979.1) | KKEFSEENVLFWLACEDFKKMQDKTQMQEKAKEIYMTFLSSKASSQVNVEG |
| | QSRLNEKILEEPHPLMFQKLQDQIFNLMKYDSYSRFLKSDLFLKAKRTEEE |
| | EEDLPDAQTAAKRASRIYNT |
| SEQ ID NO: 7/ | MFNRAVSRLSRKRPPSDIHDSDGSSSSSHQSLKSTAKWAASLENLLEDPEG |
| (NP_001005339.1) | VKRFREFLKKEFSEENVLFWLACEDFKKMQDKTQMQEKAKEIYMTFLSSKA |
| | SSQVNVEGQSRLNEKILEEPHPLMFQKLQDQIFNLMKYDSYSRFLKSDLFL |
| | KHKRTEEEEEDLPDAQTAAKRASRIYNT |
| SEQ ID NO: 8/ | MEHIHDSDGSSSSSHQSLKSTAKWAASLENLLEDPEGVKRFREFLKKEFSE |
| (NP_002916.1) | ENVLFWLACEDFKKMQDKTQMQEKAKEIYMTFLSSKASSQVNVEGQSRLNE |
| | KILEEPHPLMFQKLQDQIFNLMKYDSYSRFLKSDLFLKHKRTEEEEEDLPD |
| | AQTAAKRASRIYNT |

### AAV Vector

The present disclosure contemplates an AAV vector comprising a nucleic acid sequence that encodes an RGS10 protein. In some embodiments, the nucleic acid comprises SEQ ID NO: 1, SEQ ID NO: 2, SEQ ID NO: 3, SEQ ID NO: 4, or SEQ ID NO: 5, or a fragment thereof. In some embodiments, the nucleic acid comprises a functional fragment of SEQ ID NO: 1, SEQ ID NO: 2, or SEQ ID NO: 3, SEQ ID NO: 4, or SEQ ID NO: 5, or any combination thereof. In some embodiments, the nucleic acid comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NO: 1-5.

In some embodiments, the AAV vector comprises a nucleic acid sequence that encodes an RGS10 protein and a promoter. In some embodiments, the nucleic acid is a cDNA. In some embodiments, the promoter can be located at any position within an expression vector or cassette. In some embodiments, the promoter can be located upstream (5′) to the nucleic acid sequence that encodes an RGS10 protein. In some embodiments, the promoter can be located in a forward orientation relative to the nucleic acid sequence that encodes an RGS10 protein or a functional fragment thereof. In some embodiments, the promoter can be located in a reverse orientation relative to the nucleic acid sequence that encodes an RGS10 protein. In some embodiments, the promoter is operably linked to the nucleic acid sequence that encodes the RGS10 protein. In some embodiments, the nucleic acid sequence that encodes the RGS10 protein is not linked to a protein and does not create a tagged fusion protein. In some embodiments, the nucleic acid sequence that encodes the RGS10 protein is not linked to a protein fragment and does not create a tagged fusion protein. In some embodiments, the operably linked promoter can modulate transcription of an RGS10 mRNA. In some embodiments, the operably linked promoter can regulate transcription of an RGS10 mRNA. In some embodiments, the operably linked promoter can initiate transcription of an RGS10 mRNA. In some embodiments, the operably linked promoter can maintain transcription of an RGS10 mRNA. In some embodiments, the operably linked promoter can sustain transcription of an RGS10 mRNA. In some embodiments, the operably linked promoter can increase transcription of an RGS10 mRNA. In some embodiments, the operably linked promoter can decrease the transcription of an RGS10 mRNA. In some embodiments, the operably linked promoter can fluctuate transcription of an RGS10 mRNA. In some embodiments, the operably linked promoter can recruit RNA polymerase to enable transcription of an RGS10 mRNA. In some embodiments, the modulated transcription of an RGS10 mRNA leads to an increase in RGS10 protein levels.

In some embodiments, the promoter comprises a cell-specific promoter. In some embodiments, the promoter comprises a cell-specific promoter sequence. In some embodiments, the cell-specific promoter preferentially modulates expression in specific cell types. In some embodiments, modulated expression comprises an increase in expression. In some embodiments, modulated expression comprises a decrease in expression. In some embodiments, modulated expression comprises a repression of expression. In some embodiments, modulated expression comprises an activation of expression. In some embodiments, the modulated expression occurs over a period of time. In some embodiments, the period of time is at least 1, 2, 3, 4, 5, 6, 7, 8, 9, 10, 11, 12, 13, 14, 21, 28, 35, 42, 49, 56, 63, 70, 77, 84, 91, 98, 105, 125, 150, 175, 200, 225, 250, 275, 300, 325, 350, 365, 548, 730, or 1095 days. In some embodiments, the cell-specific promoter is a cell-type specific promoter. In some embodiments, a cell-specific promoter sequence comprises a cell-type specific promoter. In some embodiments, the cell-type specific promoter is preferentially transcriptionally active in one or more cell types. In some embodiments, the cell-type specific promoter confers an increase in transcription in one or more cell types. In some embodiments, the cell-type specific promoter initiates transcription in one or more cell types. In some embodiments, the cell-type specific promoter confers preferential transcription in one or more cell types. In some embodiments, the cell-type specific promoter maintains preferential transcription in one or more cell types. In some embodiments, the cell-type specific promoter modulates preferential transcription in one or more cell types. In some embodiments, the cell-type specific promoter initiates, confers, or maintains transcription at a higher level of expression in one or more cell types compared to one or more other cell types. In some embodiments, the cell-type specific promoter initiates, confirms, maintains, or modulates transcription that is restricted to one or more cell types. In some embodiments, the cell-type specific promoter preferentially initiates transcription in neurons. In some embodiments, the cell-type specific promoter preferentially initiates transcription in microglia. In some embodiments, the cell-type specific promoter preferentially initiates transcription in granulocyte-monocyte progenitor cells (GMPs). In some embodiments, the cell-type specific promoter preferentially initiates transcription in B cells. In some embodiments, the cell-type specific promoter preferentially initiates transcription in macrophages. In some embodiments, the cell-type specific promoter preferentially initiates transcription in brain parenchyma.

In some embodiments, the cell-type specific promoter preferentially confers transcription in neurons. In some embodiments, the cell-type specific promoter preferentially confers transcription in microglia. In some embodiments, the cell-type specific promoter preferentially confers transcription in granulocyte-monocyte progenitor cells (GMPs). In some embodiments, the cell-type specific promoter preferentially confers transcription in B cells. In some embodiments, the cell-type specific promoter preferentially confers transcription in macrophages. In some embodiments, the cell-type specific promoter preferentially confers transcription in brain parenchyma.

In some embodiments, the cell-type specific promoter preferentially maintains transcription in neurons. In some embodiments, the cell-type specific promoter preferentially maintains transcription in microglia. In some embodiments, the cell-type specific promoter preferentially maintains transcription in granulocyte-monocyte progenitor cells (GMPs). In some embodiments, the cell-type specific promoter preferentially maintains transcription in B cells. In some embodiments, the cell-type specific promoter preferentially maintains transcription in macrophages. In some embodiments, the cell-type specific promoter preferentially maintains transcription in brain parenchyma. In some embodiments, the cell-type specific promoter preferentially initiates, maintains, or initiates and maintains transcription restricted to neurons. In some embodiments, the cell-type specific promoter preferentially initiates, maintains, or initiates and maintains transcription restricted to microglia. In some embodiments, the cell-type specific promoter preferentially initiates, maintains, or initiates and maintains transcription restricted to granulocyte-monocyte progenitor cells (GMPs). In some embodiments, the cell-type specific promoter preferentially initiates, maintains, or initiates and maintains transcription restricted to B cells. In some embodiments, the cell-type specific promoter preferentially initiates, maintains, or initiates and maintains transcription restricted to macrophages. In some embodiments, the cell-type specific promoter preferentially initiates, maintains, or initiates and maintains transcription restricted to brain parenchyma. In some embodiments, the cell-type specific promoter preferentially initiates, confirms, maintains, or modulates transcription in any cell type. In some embodiments, the promoter is a pan promoter. In some embodiments, the pan promoter can initiate transcription in all cells. In some embodiments, the pan promoter is a pan neuronal promoter. In some embodiments, the pan neuronal promoter is a neuron subtype-specific promoter. In some embodiments, the neuron subtype-specific promoter is a dopaminergic neuron-specific promoter. In some embodiments, the neuron subtype-specific promoter is a glutamatergic neuron-specific promoter. In some embodiments, the neuron subtype-specific promoter is a GABAergic neuron-specific promoter. In some embodiments, the promoter initiates, maintains, or initiates and maintains transcription in microglia. In some embodiments, the promoter modulates transcription in microglia. In some embodiments, the promoter increases transcription in microglia. In some embodiments, the promoter decreases transcription in microglia. In some embodiments, the promoter represses transcription in microglia. In some embodiments, the promoter activates transcription in microglia. In some embodiments, the promoter maintains transcription in microglia. In some embodiments, the promoter sustains transcription in microglia. In some embodiments, the promoter reactivates transcription in microglia. In some embodiments, the promoter is specific for initiating transcription in microglia. In some embodiments, the cell-specific promoter sequence comprises CamKII. In some embodiments, the cell-specific promoter sequence comprises CD68. In some embodiments, the cell-specific promoter sequence comprises CMV. In some embodiments, the cell-specific promoter sequence comprises F4/80. In some embodiments, the cell-specific promoter sequence comprises CX3CR1. In some embodiments, the cell-specific promoter sequence comprises CSFR1. In some embodiments, the cell-specific promoter sequence comprises Ms4a3. In some embodiments, the cell-specific promoter sequence comprises Tmem119. In some embodiments, the cell-specific promoter sequence comprises CAG. In some embodiments, the cell-specific promoter sequence comprises mammalian IBA1. In some embodiments, the cell-specific promoter sequence comprises rodent Iba1. In some embodiments, the cell-specific promoter sequence comprises mouse Iba1. In some embodiments, the cell-specific promoter sequence comprises rat Iba1. In some embodiments, the cell-specific promoter sequence comprises human IBA1. In some embodiments, the cell-type specific promoter sequence comprises mouse Iba1. In some embodiments, the cell-type specific promoter sequence comprises rat Iba1. In some embodiments, the cell-type specific promoter sequence comprises human IBA1. In some embodiments, the cell-specific promoter sequence or the cell-type specific promoter sequence is derived from a non-human sequence. In some embodiments, the non-human sequence is a mouse sequence or a rat sequence. In some embodiments, the cell-specific promoter sequence or the cell-type specific promoter sequence is derived from a human sequence. In some embodiments, the cell-specific promoter sequence or the cell-type specific promoter sequence derived from a non-human sequence is capable of driving sufficient transcription of RGS10 in human cells of the CNS. In some embodiments, the cell-specific promoter sequence or the cell-type specific promoter sequence derived from a non-human sequence is capable of driving sufficient transcription of RGS10 in mouse cells of the CNS. In some embodiments, the cell-specific promoter sequence or the cell-type specific promoter sequence derived from a human sequence is capable of driving sufficient transcription of RGS10 in human cells of the CNS. In some embodiments, the cell-specific promoter sequence or the cell-type specific promoter sequence derived from a human sequence is capable of driving sufficient transcription of RGS10 in mouse cells of the CNS. In some embodiments, the cell-specific promoter sequence or the cell-type specific promoter sequence derived from a non-human sequence is capable of driving sufficient transcription of RGS10 in human cells residing in the midbrain. In some embodiments, the cell-specific promoter sequence or the cell-type specific promoter sequence derived from a human sequence is capable of driving sufficient transcription of RGS10 in human cells residing in the midbrain. In some embodiments, the human cells residing in the midbrain receiving sufficient transcription of RGS10 comprise microglia. In some embodiments, the human cells residing in the midbrain receiving sufficient transcription of RGS10 comprise dopaminergic neurons. In some embodiments, the human cells residing in the midbrain receiving sufficient transcription of RGS10 comprise microglia and dopaminergic neurons. In some embodiments, an IBA1 promoter derived from a human sequence is capable of driving sufficient transcription of RGS10 in human cells residing in the midbrain. derived from a non-human sequence is capable of driving sufficient transcription of RGS10 in human cells residing in the midbrain. derived from a mouse sequence is capable of driving sufficient transcription of RGS10 in human cells residing in the midbrain.

In some cases, the cell-specific promoter sequence or the cell-type specific promoter sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to that of CamKII, CD68, CMV, F4/80, CX3CR1, CSFR1, Ms4a3, Tmem119, CAG, or IBA1 promoter sequence. In some cases, the cell-specific promoter sequence or the cell-type specific promoter sequence comprises a nucleic acid sequence identical in homology to that of CamKII, CD68, CMV, F4/80, CX3CR1, CSFR1, Ms4a3, Tmem119, CAG, human IBA1, mouse Iba1, or rat Iba1 promoter sequence. Table 3 lists non-limiting examples of promoter sequences that may be used to regulate, initiate, maintain, enhance, or any combination thereof, RGS10 transcription in the AAV vector. In some embodiments, the cell-specific promoter sequence or the cell-type specific promoter sequence used in the AAV vector to regulate RGS10 transcription is at least about 90%, 91%, 92%, 93%, 94%, 95%, 96%, 97%, 98%, or 99% identical to a sequence listed in Table 3. In some embodiments, the cell-specific promoter sequence or the cell-type specific promoter sequence used in the AAV vector to regulate RGS10 transcription comprises a contiguous portion of a nucleic acid sequence that is about 90%, 91%, 92%, 93%, 94%, 95%, 96%, 97%, 98%, or 99% identical to a sequence listed in Table 3. In some embodiments, the cell-specific promoter sequence or the cell-type specific promoter sequence used in the AAV vector to regulate RGS10 transcription comprises a contiguous portion of a nucleic acid sequence that is identical to a sequence listed in Table 3. In some embodiments, the cell-specific promoter sequence or the cell-type specific promoter sequence used in the AAV vector to regulate RGS10 transcription comprises a sequence listed in Table 3. In some embodiments, the cell-specific promoter sequence or the cell-type specific promoter sequence used in the AAV vector to regulate RGS10 transcription comprises a nucleic acid sequence between 15-5522 bp in length that is about 90%, 91%, 92%, 93%, 94%, 95%, 96%, 97%, 98%, or 99% identical to a contiguous portion of a nucleic acid sequence selected from SEQ ID NO: 9-19. In some embodiments, the cell-specific promoter sequence or the cell-type specific promoter sequence used in the AAV vector to regulate RGS10 transcription comprises a nucleic acid sequence between 15-5522 bp in length that is identical to a contiguous portion of a nucleic acid sequence selected from SEQ ID NO: 9-19. In some embodiments, the cell-specific promoter sequence or the cell-type specific promoter sequence used in the AAV vector to regulate RGS10 transcription comprises a nucleic acid sequence between identical to a nucleic acid sequence selected from SEQ ID NO: 9-19. In some embodiments, the cell-specific promoter sequence or the cell-type specific promoter sequence used in the AAV vector to regulate RGS10 transcription comprises the nucleic acid sequence of SEQ ID NO: 9. In some embodiments, the cell-specific promoter sequence or the cell-type specific promoter sequence used in the AAV vector to regulate RGS10 transcription comprises the nucleic acid sequence of SEQ ID NO: 10. In some embodiments, the cell-specific promoter sequence or the cell-type specific promoter sequence used in the AAV vector to regulate RGS10 transcription comprises the nucleic acid sequence of SEQ ID NO: 11. In some embodiments, the cell-specific promoter sequence or the cell-type specific promoter sequence used in the AAV vector to regulate RGS10 transcription comprises the nucleic acid sequence of SEQ ID NO: 12. In some embodiments, the cell-specific promoter sequence or the cell-type specific promoter sequence used in the AAV vector to regulate RGS10 transcription comprises the nucleic acid sequence of SEQ ID NO: 13. In some embodiments, the cell-specific promoter sequence or the cell-type specific promoter sequence used in the AAV vector to regulate RGS10 transcription comprises the nucleic acid sequence of SEQ ID NO: 14. In some embodiments, the cell-specific promoter sequence or the cell-type specific promoter sequence used in the AAV vector to regulate RGS10 transcription comprises the nucleic acid sequence of SEQ ID NO: 15. In some embodiments, the cell-specific promoter sequence or the cell-type specific promoter sequence used in the AAV vector to regulate RGS10 transcription comprises the nucleic acid sequence of SEQ ID NO: 16. In some embodiments, the cell-specific promoter sequence or the cell-type specific promoter sequence used in the AAV vector to regulate RGS10 transcription comprises the nucleic acid sequence of SEQ ID NO: 17. In some embodiments, the cell-specific promoter sequence or the cell-type specific promoter sequence used in the AAV vector to regulate RGS10 transcription comprises the nucleic acid sequence of SEQ ID NO: 18. In some embodiments, the cell-specific promoter sequence or the cell-type specific promoter sequence used in the AAV vector to regulate RGS10 transcription comprises the nucleic acid sequence of SEQ ID NO: 19.

In some embodiments, AAV vectors based on AAV9 are advantageous for the delivery of RGS10 to the human cells residing in the midbrain. In some embodiments, AAV vectors based on AAV9 are advantageous for the delivery of RGS10 to neuron cells or microglia cells. In some embodiments, certain AAV vectors have improved delivery and expression efficiency than other AAV vectors. In some embodiments, an AAV vector comprising AAV9 provides improved delivery and expression efficiency of RGS10 to neuron cells or microglia cells, compared to other AAV vectors. In some embodiments, an AAV vector comprising AAV9 provides 1.1 fold, 1.2 fold, 1.5 fold, 2 fold, 3 fold, 5 fold, 10 fold or 20 fold expression efficiency of RGS10 to neuron cells or microglia cells, compared to a corresponding non-AAV9 based AAV vector. In some embodiments, an AAV vector comprising AAV9 provides 1.1 fold, 1.2 fold, 1.5 fold, 2 fold, 3 fold, 5 fold, 10 fold or 20 fold expression efficiency of RGS10 to neuron cells or microglia cells, compared to a corresponding AAV6-based AAV vector. In some embodiments, an AAV vector comprising AAV9 provides 2 fold expression efficiency of RGS10 to microglia cells, compared to a corresponding non-AAV9 based AAV vector. In some embodiments, an AAV vector comprising AAV9 provides 2 fold expression efficiency of RGS10 to microglia cells, compared to a corresponding AAV6-based AAV vector. In some embodiments, the expression efficiency is measured by intensity of the expression. In some embodiments, the expression efficiency is measured by relative fluorescent units (RFU). In some embodiments, the expression efficiency is measured by relative fluorescent comparison with a DAPI stain assay that labels nuclei. In some embodiments, the expression efficiency is measured by an assay described herein in Example 8. In some embodiments, the expression efficiency is measured by mean fluorescence intensity (MFI). A method of calculating MFI to determine expression efficiency may be used as provided by the following reference which is herein incorporated by reference with regard to procedures and protocols for quantitating fluorescent images using MFI (Shihan M H et al. A simple method for quantitating confocal fluorescent images. Biochem Biophys Rep. 2021 Feb. 1;25:100916). In some embodiments, the expression efficiency is measured about 1 week, 1 month, 3 months, 6 months, 1 year, 2 years, or 5 years after the administering of the AAV vector.

TABLE 3
Exemplary promoter sequences

| | SEQ | |
|---|---|---|
| Promoter | ID | |
| name | NO: | Nucleic acid sequence |
| CamKII | 9 | CATTATGGCCTTAGGTCACTTCATCTCCATGGGGTTCTTCTTCTGATTTTCT |
| (resides within | | AGAAAATGAGATGGGGGTGCAGAGAGCTTCCTCAGTGACCTGCCCAGGGTCA |
| AJ222796.1) | | CATCAGAAATGTCAGAGCTAGAACTTGAACTCAGATTACTAATCTTAAATTC |
| | | CATGCCTTGGGGGCATGCAAGTACGATATACAGAAGGAGTGAACTCATTAGG |
| | | GCAGATGACCAATGAGTTTAGGAAAGAAGAGTCCAGGGCAGGGTACATCTAC |
| | | ACCACCCGCCCAGCCCTGGGTGAGTCCAGCCACGTTCACCTCATTATAGTTG |
| | | CCTCTCTCCAGTCCTACCTTGACGGGAAGCACAAGCAGAAACTGGGACAGGA |
| | | GCCCCAGGAGACCAAATCTTCATGGTCCCTCTGGGAGGATGGGTGGGGAGAG |
| | | CTGTGGCAGAGGCCTCAGGAGGGGCCCTGCTGCTCAGTGGTGACAGATAGGG |
| | | GTGAGAAAGCAGACAGAGTCATTCCGTCAGCATTCTGGGTCTGTTTGGTACT |
| | | TCTTCTCACGCTAAGGTGGCGGTGTGATATGCACAATGGCTAAAAAGCAGGG |
| | | AGAGCTGGAAAGAAACAAGGACAGAGACAGAGGCCAAGTCAACCAGACCAAT |
| | | TCCCAGAGGAAGCAAAGAAACCATTACAGAGACTACAAGGGGGAAGGGAAGG |
| | | AGAGATGAATTAGCTTCCCCTGTAAACCTTAGAACCCAGCTGTTGCCAGGGC |
| | | AACGGGGCAATACCTGTCTCTTCAGAGGAGATGAAGTTGCCAGGGTAACTAC |
| | | ATCCTGTCTTTCTCAAGGACCATCCCAGAATGTGGCACCCACTAGCCGTTAC |
| | | CATAGCAACTGCCTCTTTGCCCCACTTAATCCCATCCCGTCTGTTAAAAGGG |
| | | CCCTATAGTTGGAGGTGGGGGAGGTAGGAAGAGCGATGATCACTTGTGGACT |
| | | AAGTTTGTTCGCATCCCCTTCTCCAACCCCCTCAGTACATCACCCTGGGGGA |
| | | ACAGGGTCCACTTGCTCCTGGGCCCACACAGTCCTGCAGTATTGTGTATATA |
| | | AGGCCAGGGCAAAGAGGAGCAGGTTTTAAAGTGAAAGGCAGGCAGGTGTTGG |
| | | GGAGGCAGTTACCGGGGCAACGGGAACAGGGCGTTTCGGAGGTGGTTGCCAT |
| | | GGGGACCTGGATGCTGACGAAGGCTCGCGAGGCTGTGAGCAGCCACAGTGCC |
| | | CTGCTCAGAAGCCCCAAGCTCGTCAGTCAAGCCGGTTCTCCGTTTGCACTCA |
| | | GGAGCACGGGCAGGCGAGTGGCCCCTAGTTCTGGGGGCAGC |
| CD68 (resides | 10 | AAACTGCCTGTTTGGGCTTCTCATTTCTTACCTCCCCTTCCCTCTCCCACCT |
| within | | GCTACTGGGTGCATCTCTGCTCCCCCCTTCCCCAGCAGATGGTTACCTTTGG |
| AF060540.1) | | GCTGTTGCTTTCTTGTCACCATCTGAGTTCTCAGACGCTGGAAAGCCATGTT |
| | | CTCGGCTCTGTGAATGACAATGCTGACTGGAGTGCTGCCCCTCTGTAAAGGG |
| | | CTGGGTGTGGATGGTCACAAGCCCCTCACATGCCTCAGCCAAGAGGAAGTAG |
| | | TACAGGGGTCAGCCCAGAGGTCCAGGGGAAAGGAGTGGAAACCGATTTCCCC |
| | | ACCAAGGGAGGGGCCTGTACCTCAGCTGTTCCCATAGCTACTTGCCACAACT |
| | | GCCAAGCAAGTTTCGCTGAGTTTGACACATGGATCCCTGTGGATCAACTGCC |
| | | CTAGGACTCCGTTTGCACCCATGTGACACTGTTGACTTTGCCCTGACGAAGC |
| | | AGGGCCAACAGTCCCCTAACTTAATTACAAAAACTAATGACTAAGAGAGAGG |
| | | TGGCTAGAGCTGAGGCCCCTGAGTCAGGCTGTGGGTGGGATCATCTCCAGTA |
| | | CAGGAAGTGAGACTTTCATTTCCTCCTTTCCAAGAGAGGGCTGAGGGAGCAG |
| | | GGTTGAGCAACTGGTGCAGACAGCCTAGCTGGACTTTGGGTGAGGCGGTTCA |
| | | GCCATGAGGCTGGCTGTGCTTTTCTCGGGGGCCCTGCTGGGGCTACTGGCAG |
| | | GTAAGGAGGAAGGAGGCTGAGGGGAGGGGGCCCCTGGGAGGGAGCCTGCCCT |
| | | GGGTTGCTAACCATCTCCTCTCTGCCAAAAGCCCAGGGGAC |
| CMV (resides | 11 | CGTTACATAACTTACGGTAAATGGCCCGCCTGGCTGACCGCCCAACGACCCC |
| within | | CGCCCATTGACGTCAATAATGACGTATGTTCCCATAGTAACGCCAATAGGGA |
| MT267310.1 | | CTTTCCATTGACGTCAATGGGTGGAGTATTTACGGTAAACTGCCCACTTGGC |
| | | AGTACATCAAGTGTATCATATGCCAAGTACGCCCCCTATTGACGTCAATGAC |
| | | GGTAAATGGCCCGCCTGGCATTATGCCCAGTACATGACCTTATGGGACTTTC |
| | | CTACTTGGCAGTACATCTACGTATTAGTCATCGCTATTACCATGGTGATGCG |
| | | GTTTTGGCAGTACATCAATGGGCGTGGATAGCGGTTTGACTCACGGGGATTT |
| | | CCAAGTCTCCACCCCATTGACGTCAATGGGAGTTTGTTTTGGCACCAAAATC |
| | | AACGGGACTTTCCAAAATGTCGTAACAACTCCGCCCCATTGACGCAAATGGG |
| | | CGGTAGGCGTGTACGGTGGGAGGTCTATATAAGCAGAGCT |
| F4/80 (resides | 12 | CAATGAAAGAAGGTGTTGAGAGATGGGAGATAGATGTTGATGGGAAA |
| within | | GAAAAAGGGAGAGACCATGGGAAGGAGAGAGAGAGAGAATGATATAC |
| DQ217942.1) | | ATTAGGCAGATTAACTTCTCTTTATTGTGAAGAAAGGGGGAAATTAG |
| | | GATTTTCTAGGCTGTCTTTTAGTTGCAGCAAGTCAGCAAAAAGGGCT |
| | | ATGATGACCAGGCAGCATTGAGCAACTCTGACTCCGCCTTCCCCTCT |
| | | TCGCACACCTTCTGGTTACTGAAAACCCAGCGTTAGTAGAAAAGTTT |
| | | CTTTTCTTTGAATGACAGAACTACAGCATAATG |
| CX3CR1 | 13 | GCAACCTCCACCTTCCGGTTTCAAGCGATTCTCCTGCCTCAGCCTCC |
| (GQ258357.1) | | CAAGTAGTTGGGATTACAGGCACCCGCCACCACGCCTGGCTAATTTT |
| | | TATATTTTTAGTAAAGACAGGGTTTCACCATTTTGGCCAGGCTGGTC |
| | | TTGAACCCCTGATCTCGTGATCCACCCACGTTGGCCTCCCAAAGTGC |
| | | TGGAATTACAGGCGTGAGCCACCATGCCTGGCCCACATTACATTCTT |
| | | ACTCACCTCCCCCTACCATGGAATTTTATTCCACAGATATGCTATTG |
| | | GTTTAGCTACTATATGTATATCTGTGTTTTATACATAAAGCACAAGA |
| | | ACCTTCCAGAACCAATTTTCGCCACCTTGGAAGTAATACCACCTCTA |
| | | CTAAGAATGCACAGCATAGACCATAAAACCTCAATGCTAAGTTCAAA |
| | | TATTGGCCCTACCACACATGAGCTGTGTGGTCTTGTACAAGTTACAT |
| | | AACTTCTCCTCCTTGTCTCAAACTCCTCACATATAAGATGAGGATAA |
| | | TAATAGTACCTGCGGCCACACACAGTGGCTTAAACGTGTAATCCCAG |
| | | CACTTTGGGAGGCTGCGGCAGAAGGATCACTCAAACTCAGGAGTTCA |
| | | AGAGCAGCCTGGGTACATGGCGAAACTCTGTCTCTACAAAAAATACA |
| | | AAAATTAGCTGGGTGTGGTGATGTGTGCCTGTAGTTCCAGCTACTTG |
| | | GGAGGCTGAGGTGAGAGGATCGTTCGAGCCCAGGAGATCAAGGATGC |
| | | AGTGAGCTATGATCATGTGGCTGCACTCCAGCCTGGATAACAGAGCC |
| | | AGACCCTGTCTGAAAGAAACAAAAACAAAAACATTAGCACCTGCATC |
| | | ATAGGGTCACTGGGGGCACTACATGAGTTCATGTACATCGAGGACTT |
| | | AGGACATTGCCTCAGGCAGACCTAGTGCTGCACAATTGCTTATGTAA |
| | | TTATTCCCAAATTTCTCCAGGGCCCACAGAAGAACATGGAAGTATCT |
| | | TGGTTTGGCAATTAAGGTGAATCACATTCTCACTCTCCTTTTCTGCA |
| | | TCTCTACCCCACATTCCCACAAAGCTTTATTCACACCAAGTCTCCAG |
| | | TCCTTGCCTGCATTGTGTGATGGGTGCCTGCAGTGATGGGTGGGGAC |
| | | ACCCATCACTGTCCAGGGCGTCCCCACCATCCTCACAGCCTCTCTGT |
| | | CTGGCCTCCTGCCTTTGAGCCAGCCCACCACACTCTCATTTCTCTGC |
| | | CCAGCAGAAACCAAACTGTCCTCTGCATTTACTGTCTCAACTGGAAG |
| | | AGAAATGCAGAATGACAAAGAACTTGTGAACAAGGGTCAGCTCCAAC |
| | | AGAGAGTGAAGCCAAAGGGGCTGGGCAGAAAGAGAGATGAAGACGGG |
| | | GGTCTGAGGAATAAGGCTGTACCAGAGTGAGAGTACGGGGGAGGGGT |
| | | TGAACAAGAGTTCAGGGAGGAGAGAATTCCCAGCGCTGAGCCAGAGA |
| | | CTCCTTTACAGAGGCCCAAGGAGGCGTGGAGGGAGGGGGAAGGCTGC |
| | | CAAGGCTCTTTCTGTCTCCATGAGTGTGTCAAGAATGCAAAGCACTA |
| | | ATGCTCTTCACTTGGTCCATCTTGCAGGGTTGAGTTTGCAGTGAGCA |
| | | ACCTTGAAGGATGAGCTGACATCTCGCTCAGGGCCAAATAACCGACT |
| | | TGCTTACTGCTTGCTATAAAATGGCACGTTACCCAAGGTCAGAGTTC |
| | | CCTTCCTATAACCTCCCCATCCCTCACACATTCACAGGTATCTATCC |
| | | AAGCCATGGCATCACTCTGTGGGGCTTGGGGGCAAGGCAACTGACAC |
| | | TGCACGCTGGTTCTCATGCTTGCCAAGCATGAAGCCCTGTGCTGCTA |
| | | GCAGCTGTGGAACATAGCCGTTAGCTTTAAAAGAGGGTAAAATCACG |
| | | TCCTGGACAGGACAGCCAGGTGAGTTGGGAAGGGAAGAGAGCCTGCC |
| | | ACGGGCACAGGCATGTTGGGGGAAGTGGAAGTGGTGAGAGCACAGTA |
| | | GGAAGTGAGAAGGGGGGGGCCGTGCTTACCAGGCCGTGGACTTAAAC |
| | | CAGG |
| CSFR1 | 14 | CCAAGTCCTCAGCTTAATTCATATGGGAAAGAAAAAAAAAGAGATGT |
| (NG_055559.1) | | AGGATCTTAGCCCAGCAGAGTTAGAGAGGTCCCAAGAATCTCCTGGT |
| | | GTGCCCGCTCCCACCAATGGACATCTGGTCTGCAGCACAGTGGGATG |
| | | GGGAGGTGCCATGCCTCAGTTTCCCCACCTATAAAACAGGGACAATC |
| | | ATCGTACCCACTCATTGGTATGTTAGGCATTTTAAATGCATGGGAAG |
| | | ACTTTAGAACATCACCTGTCACCAAATAAATGGCTCAGTAAGAGTTA |
| | | GTCACTATGTTTATTACCTAGTCTACACCCAGCAGCTCACCCCGGCA |
| | | CCCCCACACATCTGTGTTTACTGGGAAGCCAATCTCTTTTAAGGAAA |
| | | ATTTTCTTGAAGGAGAAAAGAGAACTTGAAACCCCTATCAGGCTGTC |
| | | CGGGAAGAAGGCCCGTTGCCTCTGCAGCGTCAGCATTTTTCCAGGGC |
| | | CTAAAATAGGGGCAAAGGAGGGAAGTGAGAGCTGATTCAAGCCTCCG |
| | | AGGGGCTGCCAGGCGCCCCCCAGCCTGTGTGGGCGGAAACACATTGT |
| | | TGGCGTCAGGCTCAGCCCCCGGTTTCCGCTCAGCCTGTGGCCCTTCT |
| | | ACAGGGATGTTCTCACTTCTGCTTCCCCAGCCTCCTAGCCACTCTCC |
| | | TCTCTCCAGGCAGGGAGAGGGAGTGCCACTTGCGGGGCAGCTGTGAG |
| | | GCTGGGCTCTGACGCTGACCCACTGGCTGACCCTGGCCATCCAACTC |
| | | CTCAGCTCTGGCCCTTCCTCTTCCCAGTGATGGGGGCACAATGACAG |
| | | GCCAGCCCCGAGGGCTTTCGGTGAATTCAGGGTATCTCCATTGTTCT |
| | | CTGCAAACCCCCTTGAAATGGAAATTCCCCCTGCCCCACCATAGAGC |
| | | CCAGAATTGGAAAGGTTTCCTCTCTCAAGTCCTTTCAGGATGGCTTA |
| | | ATTCACGTTTTGATTTTGTATTCATCAGAATGTGCATCCCTTCTCCC |
| | | CAAGACTCTGATCAGCAAACGAGATTGAGTGAATTGTGAATACTGCC |
| | | AAGCCTGCCACCTGGGGTTGTAGCCCTCCTATGCAGTGCAACTGCCA |
| | | GGAAGAGGGACAAGTGTCATTTCTAGCAGCCTTTTTAGCAAATCTCA |
| | | ACAGAGCTGGGGCTGCCCTATCACTGCTGCAGCAATCTAGGGCCCCC |
| | | AGGCCGGGGTTTTATAGCAGGAAACAGCTTGGAGTGTGGGCTCACAC |
| | | CAACTTGGGTTTGGATCTTGGTTCTTCCCTTACCAGCTGGGTTAGTT |
| | | TCTTAACCTCTTTGAGCCTCAGGCTATTCTTCTGTCTGATGGGAATA |
| | | ATCCCAGCTCCCACCCCAAAAACTTCAGCTGAAGGTTGAGTGAGATG |
| | | ATGTATAGAACTACTCCTGCCCTGGGCACCTGCITTAATTGCTACAT |
| | | TTCTAATGTCCCTTCTGGGCCAACTCTGATCGCTCAAGATTTTAACC |
| | | TTGGATTCTGTGATTCCATTTGTTCCTTTTTTACACCTGCCTCACAC |
| | | CTTACAGTGGGCTTCTCCCATCCCTTCCCCCAGCCTGTCTCTCTAGA |
| | | CCCTGCCTTGGCCTCCAGGAAATGTGGTCAGGGAGCGGTGTCTGCCC |
| | | TATTTTGTCCTATCCTATTGTCCCAGGTATTCACGCAGGATAAGGCT |
| | | GATACCCGCAACTTCCGCAGCCAACTTCCCCTGCTGGCTTGGCTAGC |
| | | AGGGAAGGAAGCCGGCTTGGCCCCCATCCTCCTCGGGGTGGGGGCAT |
| | | GGGCTTATGAACATTAACTGAGGTCCCCTTCTGTGCCTGTCCATTCT |
| | | TCCCCCCTCTCATCTCCACTCCCCAATTCTCCCCATGCCCCACACCT |
| | | CTCCACTAGCTCCCACTTGCCTGGAAGCCCCTCCCTCATCACTTCCA |
| | | GAATGTGCCCCAATCTCTACTCTTCTCTCCCAACACACACACACACA |
| | | CACACACACACACACACACACACACACACACACACACTGCACACTAA |
| | | GGCTGGACACAGCCCCTCTTCAGCCCAGGATCTCATCGCAGTAATGA |
| | | AGAGAGAGCCCCCCTCTCTCTCTAGGGTCCTCCAATACCATCCCCAG |
| | | TCTCCTCACAGTGGCCCCTCATCCACTCATCATTGGTCTGTTGGGCT |
| | | GAAAACTTAGGTTTCCAGCCTCCCCATCTCTTCTTTCTCTTGGGACT |
| | | GGACCCTGGAAAACCATCTGGCCCCCCTCCCACACACCATGCCCCTG |
| | | TGAAGGGAAAATAAAAACTTGGGACCCTAATTCACTCTGCCAAAAGG |
| | | AAAAATCAAGCTGGAAACTGAGTCATGCAAGAAGCTGCCTTGCCTTT |
| | | TATTCCTAAGCAGATAGCGACAGATAAAAGGTAAAATATCCCACAGG |
| | | CAGCTCCTCTATGTTCACTTTCCCTATGTAAAGAGCCAATTTACTGA |
| | | ACCGAGATAATTGACTGTTCCCCTACCTGCTGCTTTTCTTTTGCAAC |
| | | ATGTGGATTACCACAACCTCCCCTTTTCCCCTCCAGCCCACTTTCCC |
| | | CTTTAAATATTAAAGCCCTCGAATTCATCCAGAATGGCACAGACCAC |
| | | AGAATGTTTTTGTGACTCCATGTTTTTTTCTCGCCAGCATGTCCTTA |
| | | ACCTTGGCAAAATAAACTTCTAAATTGATTGAAGCCAGGCACAGTGG |
| | | CTCATGCCTGTAATCCCAGCACTTTGGGACGCCAAGGCAGGGGGATC |
| | | GCTTGAGTTCAGGAGTTCGAGACCAGCATGGCCAGCATGATAAAACC |
| | | CTGTCTCTACCAAAAATAAAAAAATTAGCTGGGTGTGGTGGCAGGCA |
| | | CCAGTAATCACAGCTACTCAGGAGTGGCAGAATTGCTTGAACCCCGG |
| | | AGGCGGAGGTTGCAGTGAGCCGAGATCCATGCCATTGCACTCCAGCC |
| | | TGGGCAACAGAGCAAGACTCGGTCTCAAAAAGATAGAAAAAAGAAAG |
| | | AAAGAAAGAAAGAAAGAAAGAAAGAAAGAAAGAAAGAAAGAAGGAAG |
| | | GAAGGAAGGAAGGAAGGAAGGAAGGAAGGAAGGAAGGAAGGAAGGAA |
| | | GGAAGGAAGGAAGAAAGAAAGGAAGGAGATGGAGTCTTGCTCTGTCG |
| | | CCTAGGCTGGAGTGCAGTGGCATGATCTCAGCTCACTGCAACCTCTG |
| | | CCTCCCGGGTTCAAGTGATTCTCCCACCTCAGCCTCCTGAGTAGCTG |
| | | GAACTACAGGAACCCACCACCACACCCGGCTAATTTTTGTATTTTTT |
| | | AGTAGAGAGGGGGCTTCACCATATTGGCGAGGCTGGTCTGAAACTCC |
| | | TGATCTTGTGATCCACCCGCCTCGGCCTCCCAGAGTGCTGGGATTAC |
| | | AGGTGTGAGCCACCATGCCTGGCCACAAGACTCCATCTCAATAAAAA |
| | | AAATAAAATTAAATTGAGACCTGGCTCAGATACTTTTCCGTTTTTGC |
| | | CCCACAGGCAGGTTGTGGGTGTAAGGATAGGACAAGGCAGTGAAATC |
| | | AAACCTGTGGGACTCTCTGAGATGAAGGGGGTGGCGGGTACACTGTG |
| | | TGTGCATTTGTCAGACCTCATTGAACTTAAGAACCTGTGCTTTCTAC |
| | | TGCATGTAAGTTACATTGCAAAATAAGAAGTCCAGGCCGGGCGCCGT |
| | | GACTCACACCAGTAATCCCAACACTTTGGGAGGCCAAGGCAGGTGGA |
| | | TCACCTGAGGTCAGGAGTTCGAGACCAGCTTGGCCAACATGGTGAAA |
| | | TCCCATCTCTACTAAAAATACAAAAATTAGCCAGTCATGATGGTGTA |
| | | TGCCTGTAATCCCAGCTTGGGAGGCTGAGACAGGAGAAACACTTGAA |
| | | CCCGGGAGGTGGAGGTTGCAGTGAGCCGAGATTGTGCCACTGTACTC |
| | | CAGCCTGGTGACAGAGAGAGACTCTGTCTCAGGAAAAAAAAAAAAAA |
| | | AACCCAAATACTCAAGCTCAGTGTGATTGCCTGGGCCAGAGAAGGAT |
| | | GGTCAGTGGGCCATCTCCTCCTCATCAGGGACCAGAAGCGCCACGGC |
| | | CTTAGGGTCCTTCTGTGAAGAGGCCAGTATGAGGCTGTCCTTTCTCC |
| | | CCTTCAATCCCCATCCTCCCCCAAGGTCCCTTTGAATAAGCAGGCAA |
| | | TAGAAATGTGTTTGTTGGGCCAACAAGCCAAGCACAGCAGCCCCTCA |
| | | CCTCTAGTCATGTTCCCTCCCAGATGGAAATTCTGCAGAACTGCTAC |
| | | AGCCTGGGGACCTCCTGAGACTTGGCAGCCTGTGTCTGGGAGGGCTC |
| | | TGGTCCAGGAGACCCTCACTGGCCTCTTCCCAATCCCTCCTCTAGCC |
| | | CTATACCTATTTCACCCCGAATTCCCCTCTCCTGAGCCCTGGGCCAG |
| | | AGAGGCCAACCTCTTGTCTCTCCCCTCTGACCCCTTACACAAGCCAC |
| | | CCAAGGAAGAACCACCACCCCTATCAGGCCCTTCCAAATCAGATCTT |
| | | GCATAGAATCATCGAATCACAGGCTGGCAGAGCTGCCAGAGCCATCA |
| | | GGAGCTCTCCAGGTCCTCCCAACTCAGTGTAGGTCTGGACCAGCAGT |
| | | GCCGGCATCTCACATCTCCCAGGAGTGTGTTGGATATACAGAATCTC |
| | | AGGTCGCACCTGAGACCGACTGAGTCTGAATCTGCATTGTAACAAAA |
| | | TCCTCAGGTAACTTGTATGCACAGGAAAGTATGAAAAGCACTGCTCT |
| | | GGTCTAGTCTATCACTGTCCCCCTGCCCCCAACACCTGCAATTGAAT |
| | | AGAAGTGGACACTGAAGCCTCAAGAGTATCAGAGGCTTGCTCAGGAC |
| | | ACACAGTGAGTCGCTGGCAGAGCTGGGACAGATCCACAGCACCAGGT |
| | | GGACCAGTTCCACCACACCCATCTCTTTTCACCCCACTACCTCTCCA |
| | | AAGCAGATACCCACAAGCCTGCAGTCCAGTGTTGGGGAGACTAAAAC |
| | | AGCCTGAGGACACCCAGTGAGGGGCAAGGACTGAATCCTTTCACAGG |
| | | GGTCTTCTCCATCACACCCCAACAAAGTCCCCCACCCCCCGTTCTGC |
| | | TCTTACCATGCCAAGCTGTGGCCACCAGCAGGAGCAGCAGAACTCCT |
| | | GGGCCCATGGCCTCGGTGGGGAAGTGGCAGGCAGGTGCAGGGCTGCA |
| | | AGGTGCCCAGGGCACAGAGCTCTCAGCTACTAGCTCCGCAGGGATCG |
| | | GGACACTGGACACACGTTCCTCTCCTCTGCACTGGCTGTTTGTCTTG |
| | | TTTTCCTCTTCCTCCTCCTTGGGCTGATCCTCTTCTTCCCCTTTTAG |
| | | CTAGGCAAGGCAACCACAGAGTTTGGAAATCTTGGGTCTTTAAGAAG |
| | | AAAACAAAGCCTTGAACCCAAGAGGGAGGGGTCGCAGTCTGGGGCAG |
| | | GGCCAGCCTGAGCTTGGGGGGAGCTAGCTAAGTTTGGGGCCCTTCTA |
| | | AAGCCATCTTCAAGGGGTCTTGGGAGAAGAGACATGTCCAAGAGCAG |
| | | CCAGACCGTTAGTCAGTAGCACAAATGAGGGTCTTCAGAGTGATCAG |
| | | GTGGCTCTGGCAGATCCCAAAGAGAAGTTAGGTTGCATGATATTGTG |
| | | ACATATATATTTTTTCTTTACCCTGATGTCCTGGCTTACAACTCCTA |
| | | AAATCCTTGGAATCTCCAAAGTA |
| Ms4a3 (resides | 15 | TTGTTGCTTGTGTTTCCTGTTCCAGCAATGTGTTAAGCAACATCTGAAAGCC |
| within | | AGAAGACAGTTATCCCTCACACCAGTTTCCTAATCATCACTAAGCCCTGGGG |
| AP000790.4) | | AAACACAATGGATCAGCGAGGCCCCAAATGCAGATGTGATTAGCACACTGGT |
| | | CAGTCAGCCCGCAAAACACTTTCACCAAGTCCTCTAAAGTGTTGCATAAGGC |
| | | AAAGATAACGTTAAGTCTGTGGAGGCTTCCGTTATCGGGAAAAGATGCTGTA |
| | | GTGATCTTTTCTGAGTGTCTCCTACTTGCGACAAGGTGGACTTGGGAGGAAA |
| | | GCCGTCTGCCAAAGCCTGAAGCCTCCAAGGTATAGATTCCCTCTCCATAGAC |
| | | ATTCCCCTTTCTTTTATATCTTTCGTTAAAATGCCTCCCAGTGGACTACTGG |
| | | GGATGTCTGCAAAGGTCTCTGTTTGGTTGCTATGTAGAGGTTTGGAACATTT |
| | | AACTGTGAACATGGCTTCTCTTTAGGATGTGATGATTCCTTTGGAGCCATGA |
| | | TAAAAAGTGTTAGACATAATAATACAGCAATTTCTTTGGGAAAATGTGGGTA |
| | | TGGATACACACACATGCACACGCACCTCTTAGGTACATTAAATCCCACTGGC |
| | | TGCACCTGAGTTTGTCTGCCAGCTCTGATGATGAGGGGTGGTCTCGATATGC |
| | | CTGAAGTATGGGGTCTGCAAAGTGCATTTTAGCATAGCTCAGGGGACAGTGG |
| | | AGTGTAATGATAGCTTGAGGTTCTGATTTTTAGCTTCTCAAATTGCTGGCTG |
| | | TGTGGGCTTGGGAAAGCTACTTAACTTCTTTGTTCCTCAGCTTCTTCATCTG |
| | | TTAGATGGAAAGGATCATAGCAGTAGTACCCATTCATCTGATGTTGTGAGAA |
| | | CTAAATTAGTAAACATGGATAAAGCCCTTAGAACAAATGCCTGCCACATACT |
| | | AAGAGCTGGAGAGATGAACTTAACTGGCAGTTTCCTAGAAAATCTACATTTC |
| | | TCTGATTTTGTGTGTGTGTGTGTGTGTGTGAGAGAGAGACAGAGTCTCGCTC |
| | | TGTCACCCAGGCTGGAGTGCAATGGCACGATTTCGGCTCACTGCAAACTCCG |
| | | CCTCATGGGTTCAAACAATTCTCCTGCCTCAGCCTCCTGAGTAGCTGGGATT |
| | | ACAGGCGCGTGCTGTCACAACCAGCTAATTTTATGTGTTTTTAGTAGAAACA |
| | | GGGTTTCACCATGTTAGCAAGGATGATCTCAATCTCCTGACCTTGAGATCCT |
| | | CCCGCCTCTGCCTCCCAAAGTGCTGGGATTACAGGCATGAGCCACTGCACCC |
| | | GGACCATTTCTCTGATTTTTTTAAAAATAAATAACTGATTTAACCAAATTTT |
| | | AGAAAACATAGTTTGTTTCACCTGGAGGCTGAAAATTCTCCCTGATTATT |
| Tmem119 | 16 | CACATGCACACACCACACATATATGCATACACTCCACATACACACAACCATA |
| (resides within | | CACATACACCACACACACTCACCTACACAACCACACACACATGCACACACCA |
| AC008119.6) | | CACACTCTGAGCCAGGAATGGCTTGTTCCCGCTGGTGGACATCACCTGTCAG |
| | | ATGCTAAATGAGGAAAGGACCCCCTCACCTCTCTGGCACCTGTTAGCAAAGG |
| | | TTCCTACAGACAACCTTGAGCTTTGGGGTCTGTGACCTGCCAGTCACCTCCC |
| | | TGCCTCCCACCCCCAGTCAAGCCTAGGAGAGAGCTTTTGTGGCAACAACTAC |
| | | CACTTCTCACTGCAGAGGGCAGCTGGGACCATGGGTGGCTGGAGACCCACTC |
| | | AAGTTCTGGCTGGGTCAAGAATCCCGGCTCCATCGCATCCAGCTCTGTGGTC |
| | | TGGCTCCTGTGGGCCTCACTTTCCCCATCCGTAAGTTGGGACAGGGTGACCA |
| | | CAGGTAGCACCTCTCAGAGCTGTTAGGGCCAAATGAGACAACTTTCAGAGAG |
| | | TGCCTGGTGCGAAGTAGCTGAGCCCCGAGGAAAGATAGGGTCTGGGGTAGCT |
| | | GAATTCCTTCCCTAGCCGAACTCCAGGTTCAGCCAGCAATTTCCCACCCTGA |
| | | GTCGACAAATGAGCACCATCGCCGGATGGGAACATGCGCTCACTGTCTGGTC |
| | | CGGCCGCCACCCAGTGAAAGACCAACCTTTTCACCCCGTTCAGTGAAACCTC |
| | | TTTAGCTCTGGCCGCCAAGGAATGCAGAACCAAACACACACATGCACACACA |
| | | TACACATACACACACATGCACACATACACCTACACACACATATACATACAAC |
| | | ACACACACACACACACACACGAAGTCAGAGGCAGAGGGCAAGAAAGGAGAAC |
| | | ACACAAACTGCCACTGGCTCTTTAAGGCTGAGACCCTGACTCTCAGAGTCCC |
| | | CTGCACCCCACAACCCCTCCTGGCTTCCAGGGAAAGGTGGGGGCAACGTATG |
| | | GAGGACCCAGCCCCACCTGCAGTCAGCAGAGCCTCAAAAAAGATGGTTGTGT |
| | | GGCCCTGGACGGCCCCTGCCCTCTCTGGGCCTCAGTTTCCCATCTGTGGAAT |
| | | GAAGCACTGTTCTGGGCAGCTCTGAAGTCATTATTCTGCCTACCTCCCCCCG |
| | | GACCCTAATTTTTTCAAGGACTCAGAATCTAACAGGCCATTAGGAGCTGGGG |
| | | TCAGGCCCAACGCCTCTTGCTTCACCTTAGAGGTAACTCAGGTCCAGAGACT |
| | | GGAAACAGCCTGCCCAGGTCACACAGAAAGTCAGGGGTGAGCCCAGCAGAGC |
| | | CCTGGACAGAGAAGCCTGTCTTGGTATATCCCAGAGGAGGTCACTTCCCGGC |
| | | TATGTGTCCTTGGGCTGGGCATGTCACCTCTCTGAGCCTCGGTTTCCATATC |
| | | TGTACAATGGGGATAAGAGCCACCTCCCTGGACTGCTGTTAGGACAAGATGC |
| | | GGCCATGCCTGGTGCACAGTGGGTGCCAATTAGCAGGTACCACGTGATTGCC |
| | | CAGGACTCCCTGCCTGGGGACATGTCAGGCAGGGCTGGCCGTGTGTAAGTGT |
| | | GGCAAATGCCCTAAGATGGTCCTGAGTCTGTGGAGGAGTGCAGATGCACCCC |
| | | TGAGGCCTGGGGGCCCCAAGACTTGAAGCTAGGGGACCGGGGGCTGCCCCAC |
| | | CCCCACCCAGAGACTGGTGGCCTTTGGGGGAGGGGCGGCCCACGGAAACCTG |
| | | GCTTCAATCAGTGGCCTTGTGGGGGAGGGGTGTTCTGGTTGGAGCACTGCAC |
| | | ATGCCCCCGACCCCAGGGCCAGCCAAGGACAGGGACCTGGGCCCTGGAGGTC |
| | | TGGATGGGGAGGGGGGTCCTGGGGGCACAATCCCCCTCCACACACACACACA |
| | | CACACAGAGCAGCATGGACACACAATATAAAGACACACAACACAGGCACACC |
| | | GACTCACACAGCACTGAGACACCCAACAGCACTCTCATGGCCCATGCACACA |
| | | ATACACAGAGAACACACACGGACACACAACACATATACATACACAACCCCCT |
| | | GACACACAGCCGATCCCTGCACACAGGACAATACCGTCCACATCACCCTCAC |
| | | ACAGATCCTGGAAGCAACAGCCTGGCATCCACTTGCAGAAAAGTGGAAGCAA |
| | | CACACCCCCTACACCAAGGCCCCAGCCGCACACACACAACACTCCGGCCCAC |
| | | CCTGGCCCACCCCAGCACCGCACACGGGACTCAGATTCCACCACACTCATGC |
| | | ACACAACACCAAACACACACTCAAAGCCCACACACACTTAGAGAGGCCCCCC |
| | | AGAGTGCTCCAACAGTCCCCAGTCCCTAAGCTCCAAGATAGCTCCAGGCTCT |
| | | CAGGACAGAGGAGGGGACCCCAGGACTCACCAGTTCCTTGGCGTACAGGACT |
| | | GGGTGCTCCCGCCGAATGGAGCGAGAGTGGGGGGCACCTGGGCTGGAAGAGA |
| | | GAGTCCGTGCTGGTGCCCGGGGCTGCTCTGGGCTGGCGCCTGCCCGCCCTCC |
| | | CTCCTCCCGTCCCTCCTCCCGTCCCTCCTCCTTCCTCCCTCCCCGGCTGGGC |
| | | CGCAGGGCCAGCTTGATTTCATTAAGAGGGTTTGGGGTGGGGACAGGGGGAG |
| | | GGGAGGGGCTACTCTCGGGCCACACGCAATCCCACCCTTGCTCACACTCTCG |
| | | CTCTGGGGAAGACCAGATGCGGCACTTCCTCCCTGACCTCGGGTCCCCAGGG |
| | | CAGGCTGAGACAGACAGACAAGGCTGTCCTGCAGCGCTCCCCCAGACAGTGA |
| | | GCTGGGGTTCTGCCCACGCTGCTGCCCAATACCCTCCAAAGGGGACAAGCTC |
| | | AGGGAGGTTCAGAGGCCTCTGGAAGTGGGAAGGAAAATTCCCCGTCCTTGTG |
| | | TGGCTCAGGCTTCCAGGCTCAGGGTGCAGCGTGGGAGCTGGGATTCGTTTTA |
| | | GAGACAGGCCTGATGGTGGGTCCGAGCCTGAGACAGGGAGCCCAACTCTGCC |
| | | CTTCCCTCTCCAGACCTCAGTTTCCCATTTGTAGGGAT |
| CAG (resides | 17 | GCGTTACATAACTTACGGTAAATGGCCCGCCTGGCTGACCGCCCAACGACCC |
| within | | CCGCCCATTGACGTCAATAATGACGTATGTTCCCATAGTAACGCCAATAGGG |
| LT727314.1) | | ACTTTCCATTGACGTCAATGGGTGGAGTATTTACGGTAAACTGCCCACTTGG |
| | | CAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTT |
| | | CCTACTTGGCAGTACATCTACGTATTAGTCATCGCTATTACCATGGTCGAGG |
| | | TGAGCCCCACGTTCTGCTTCACTCTCCCCATCTCCCCCCCCTCCCCACCCCC |
| | | AATTTTGTATTTATTTATTTTTTAATTATTTTGTGCAGCGATGGGGGCGGGG |
| | | GGGGGGGGGGGGCGCGCGCCAGGCGGGGGGGGGCGGGGCGAGGGGCGGGGCG |
| | | GGGCGAGGCGGAGAGGTGCGGCGGCAGCCAATCAGAGCGGCGCGCTCCGAAA |
| | | GTTTCCTTTTATGGCGAGGCGGCGGCGGCGGCGGCCCTATAAAAAGCGAAGC |
| | | GCGCGGCGGGCG |
| human IBA1 | 18 | ATATCTGTTGAGTGAACGTATGAATAAAGAAGTGAGTTCCTCCCAGC |
| (resides within | | AGGCACTGAGAACATTGGGAGTACAGGGTTGCAGCTCTCTCTGCAGC |
| BX248305.13) | | AGGAGAATGTAGCTGCAATAAAGGGAAGTCAAGAAGCCAGAGTCCAG |
| | | CCAGGTGCAGTGGCTCATGCCTGTAATCCCAGCACTTTGGGAGGCTG |
| | | AGGTGGGTGGATCACAAGGTCAAGAGATAGAGACCATCCTGGCCAAC |
| | | ATGGCGAAACCCCATCTGTACTAAAAATACAAAAATTAGCTGGGCGT |
| | | GGTGGTGGGCGCCTGTAGTCCCAGCTACTCAGGAGGCTGAGGTAGGA |
| | | GAATTGCTTGAACCCAGGAGGCAGTGGTTGCAGTGAGCCGAGATTGC |
| | | ACCATTGCACTCCCGCCTGGGCGACAGAGCAAGACTCCGACTCAAAA |
| | | AAAAAAAAAAAAGCAGCAGCAGCAGCCAGAGGCCACTCCAGCATCTC |
| | | CCCTACCTGGCTTGGGTCAGGGAGAGGGCAGTGAGAAGTGAAAACTC |
| | | CCAGCTACAGAAAAGGAAATATGTTGGGGGGAAGGGAGAAGGAAAGG |
| | | TGTCTTCATCAATGCCGGGGCAGGGTAGATGGAGCCCTGGGCAGGGA |
| | | GTTTGGACCAGGAAATCTCAATGAGGGAAATGTGCTGTCCTCACCTC |
| | | TCCAAGAAGCGACTGGCCAAACAGAGTGACAGAGGGGATAAAGGTTA |
| | | TGCCTAGGGAGGCATGTGTCAGAGGCTATCATCCACTCTGTTGAACC |
| | | CACAGTGACCAGCACCACCATCACACAAACATGCCTGCATGTGTGCA |
| | | CGCACGTGCAGTGTGCAAACCTGATGTCAGCCTCACTCCCTGGCTCT |
| | | TCTGTCCACAAACGCTGTTTCTTTAAGTACCACTTTCAGTTCCTCCA |
| | | AAGAATCTACTTAAACTCTTAAATTCCTGATCTCTATAGATTTTACT |
| | | AAAGATTTCAAAGGAGATAAGATGAGAGGGTTACGTTGCACATTCTA |
| | | AAGCAAACAAATTAAAATGTTTTGTTAGACATTTCCATATTTTTAAG |
| | | GGCCTCCTTGGAGCTGCCAGGCTGGGAGTGAGGTTTCTCTCCCTTTC |
| | | TAAACCCTGTGCCCATCTTGTCACCCTCCTGGAGCTGCCAGCAGACT |
| | | TCAGATTCTTCTCCGATCTACAGAGCAGAAAAATTCAGCCAGCCCTT |
| | | CCTTGTCTTCCTATCCACAGCTGCCTGCCCAGACTCATGAAACCTGA |
| | | CAAAATGCAAGGTCTTATCATTACCTGAACCTTGGACCTGTTCAAAA |
| | | ATACTAGTTCCTGAGAATAAATATCCCTGGTGTCTTCCTGCCCTTCC |
| | | TGCACACCTCCAGTGGCTTATCAAAATATTTGTTTCATGCGCACACT |
| | | GGGCTCTCATTTAAGAGGAATTTGGGAGAATGTTATTTTCTAATCTG |
| | | CATTTCACACCAGGCTCCCCCTCCTTCCTGGGGTGCTAGTGTCAGCA |
| | | GAACCTGATGGGGAAGTGAGGTCTGGGAGGCAGAGGAGGAAGGAATG |
| | | AGGGGAAAGGGGAAGTTTGGGAGGAAGGCTTCTGAGAAGACTGGTGG |
| | | GAGAGAAGGAGAGCCTGCAGACAGAGGCCTCCAGCTTGGTCTGTCTC |
| | | CCCACCTCTACCAGCATCTGCTGAGCTATGAGCCAAACCAGGGATTT |
| | | ACAGGGTAGGGAGGGTGGGATAGGCAGCGGCATTAGATCGGAGGAAT |
| | | GAGATGGACAGACCTGGGCTGTGGGCTAGGAGGGCAGTCAGCTGGCC |
| | | TAGGGTAGCCCGGGCTGGTGT |
| mouse Iba1 | 19 | TACTATAGGATGCATCGTGAAAACCTCGTTTCCACCAGGAACTGAGG |
| (resides within | | TTGCTGCTGGAGGAAATCTCCCATGAAGAGCTGTTAACTGGGGAACT |
| AB036423.1) | | GGTTGGATCCAGCTCCTACAAGCTCTCCTTTAATTTCCATACACATG |
| | | CCAAGCTGTGAACACCTGCACACACAGTACAAATAAAACAGTAGAAA |
| | | TGAAATGAAAATTAAAACAAAAACAGCATACTTTCTCCTTTCTACCC |
| | | ACATATCTCCTGCATCCCTGAGAGCAATTTCTTGGCCATCTTCCATA |
| | | ATCATGCTATTCAAACCTGCCCTCTAAGTACAGAGCTCTGAATGGAG |
| | | ACCACGGGAGCAGACTTTTCATCTGTTGGCTCCCTGGGCCTAGCAGT |
| | | GTACCCTGCAAACAGGAAACCTTCGCTCATCATTTATCACATGAATG |
| | | TGGCTAGAGAACTGTCCCCACCAGACACTGAGAGCCTCTCACAGGGC |
| | | CAGAGGGTGGCTGCCTCAAAGGCAGGCGAGAGGCTGCAGTCTTCTGC |
| | | AGAGTGGACAGATACTGCCTGCCATACAAGGGGACATAGCCTGGGGC |
| | | GGGGGGCACAAGGGAAGGGGCCTGAGTGCATTGGAGTCACAGCCCTC |
| | | CCTCCCCTGGTGACCCAGTGACTGAGGGGACCAAGGCTATCCCTGGT |
| | | ATGAGGGGAGGGCATTTGCATGAGTTAGACCCCTGACAGCCCATGGT |
| | | GGGGACAGGAAGTAGCTCTGTCTACTGTCTCTTTCAGTCTCACTTTC |
| | | TGTTCCTCCAAAGTGTCTCTTCAAACTCTCAAATGACTTTGTAGATT |
| | | CAGCCTAAGATCTCAAAGCAGGTGAGGCAGGAGGCTGGCACTCAAGA |
| | | GCCTGACGAACACAAACCCAAGTCCTTCTGGGACATGTCTCCATGTG |
| | | TCCCCAGTGCTCCTCTGTAGCCCCTGAGAGAGGTTTCTTTCCTTTCC |
| | | AGGGCGCTAGGCTCAGCTCACCCCATTCCTGGAGCAGCCTGCAGACT |
| | | TCATCCTCTCTCTTCCATCCCGGGGAAAGTCAGCCAGTCCTCCTCAG |
| | | CTGCCTGTCTTAACCTGCATCATGAAGCCTGAGGAGATTTCAAGTAA |
| | | ACCCTCCCAAGCCCCACCTCAGGATCTGGGGAAAGCCACTGTCTACC |
| | | GCATCCTTGGTTTGAGACAGGTTCTTACTGTGTTGGCCTGGCTGGGT |
| | | TCCAACTCCCAGGATCCTCTAGCCTCAGCCTCTCTTAGGTGCTAGAT |
| | | TGCAGGCCTAGGGTATCACGCTCGGTCCCTCAGATCCTGGTGCCTTT |
| | | CCACACCTCCGGGAGCTGATCTAAGTCTTTCTCCCACGTACAGTCTG |
| | | CTTCTTTGGAGGAGCTGTGTGGAGCTAGGTGTGTTACAGGGCTGTAG |
| | | CTCCAGCCATCTGGGAGGCTGAGGCAGGATGGCTACTTCAATTCTCA |
| | | AGAGCCTGGATAGGAGACCCTGCCCCCACCCCACCCCCAAAGTAATT |
| | | TTTCAGCACATTACTTCTTCATCTCCTCTCTCAACCCGTTCTGCCTT |
| | | CTCCTGGGGTGCTGGTGTCAGCAGAAGCTGATGTGGAAGTGATGCCT |
| | | GGGAGTTAGCAAGGGAATGAGTGGAAAGGGGAAGTGTGAGAACGGTC |
| | | CCAGAAGAGACTGGGGAGCTGGTGGAGAGAGGACCCAGCGGACAGAC |
| | | TGCCAGCCTAAGACAACCAGCGTCTGAGGAGCC |

In some embodiments, the promoter comprising a cell-specific promoter sequence is operably linked to a nucleic acid sequence that encodes an RGS10 protein or a functional fragment thereof. In some embodiments, the promoter comprises a length of between 15-5522 bp. In some embodiments, the promoter comprises a length of between 15-3002. In some embodiments, the promoter comprises a length of between 15-2025 bp. In some embodiments, the promoter comprises a length of between 15-1760 bp. In some embodiments, the promoter comprises a length of between 15-1700 bp. In some embodiments, the promoter comprises a length of between 15-1678 bp. In some embodiments, the promoter comprises a length of between 15-1402 bp. In some embodiments, the promoter comprises a length of between 15-1289 bp. In some embodiments, the promoter comprises a length of between 15-821 bp. In some embodiments, the promoter comprises a length of between 15-584 bp. In some embodiments, the promoter comprises a length of between 15-508 bp. In some embodiments, the promoter comprises a length of between 15-315 bp. In some embodiments, the promoter comprises a length of between 1600-2025 bp. In some embodiments, the promoter comprises a length of between 1678-1760 bp. In some embodiments, the promoter comprises a length of between 15-1500 bp. In some embodiments, the promoter comprises a length of between 15-1000 bp. In some embodiments, the promoter comprises a length of between 15-500 bp. In some embodiments, the promoter comprises a length of between 15-300 bp. In some embodiments, the promoter comprises a length of less than 200 bp. In some embodiments, the promoter comprises a length of less than 150 bp. In some embodiments, the promoter comprises a length of less than 100 bp. In some embodiments, the promoter comprises a length of less than 75 bp. In some embodiments, the promoter comprises a length of less than 70 bp. In some embodiments, the promoter comprises a length of less than 60 bp. In some embodiments, the promoter comprises a length of less than 55 bp. In some embodiments, the promoter comprises a length of less than 50 bp. In some embodiments, the promoter comprises a length of less than 45 bp. In some cases, a promoter sequence operably linked to the nucleic acid sequence that encodes the RGS10 protein or a functional fragment thereof drives high expression in microglia. In some cases, a promoter sequence operably linked to the nucleic acid sequence that encodes the RGS10 protein or a functional fragment thereof drives high expression in cortical neurons. In some cases, a promoter sequence operably linked to the nucleic acid sequence that encodes the RGS10 protein or a functional fragment thereof drives high expression in neurons of the dorsal striatum. In some cases, a promoter sequence operably linked to the nucleic acid sequence that encodes the RGS10 protein or a functional fragment thereof drives high expression in hippocampal neurons.

In some embodiments, the promoter is operably linked to an enhancer element. In some embodiments, the enhancer element regulates transcription of the nucleic acid sequence that encodes an RGS10 protein or a functional fragment thereof. In some embodiments, the enhancer element mediates cell-type specific expression of the nucleic acid sequence that encodes an RGS10 protein or a functional fragment thereof. In some embodiments, the enhancer element mediates RGS10 protein expression in microglia. In some embodiments, the enhancer element mediates RGS10 protein expression in neurons.

The combined length of a promoter sequence and an enhancer sequence of this disclosure can still be short. The combined promoter and enhancer can have a total length of about 50 to 600, 100 to 500, 50 to 300, 100 to 250, or 150 to 300 base pairs in length. The combined promoter and enhancer can have a combined length of less than about 600, 500, 450, 350, 300, 250, 200, 150 or 100 base pairs in length. In some cases, promoter is 38-50 bp, 45-50, 49, 50-63, 56, 50-59, or 45-60 bp.

In some cases, a promoter sequence and an enhancer sequence are combined directly with no additional linker sequence. In some cases, a promoter sequence and an enhancer sequence are combined with one or more short linker sequences which can be either deliberate or cloning artifacts. In other examples, the combined promoter sequence and enhancer sequence can include short sequences, generally less than 50 bp or less than 15 bp, from cloning plasmids or restriction enzyme recognition sites. In some cases, one or more promoter sequence and enhancer sequence operably linked to the nucleic acid sequence that encodes the RGS10 protein or a functional fragment thereof drives high expression in microglia. In some cases, one or more promoter sequence and enhancer sequence operably linked to the nucleic acid sequence that encodes the RGS10 protein or a functional fragment thereof drives high expression in cortical neurons. In some cases, one or more promoter sequence and enhancer sequence operably linked to the nucleic acid sequence that encodes the RGS10 protein or a functional fragment thereof drives high expression in neurons of the dorsal striatum. In some cases, one or more promoter sequence and enhancer sequence operably linked to the nucleic acid sequence that encodes the RGS10 protein or a functional fragment thereof drives high expression in hippocampal neurons.

A promoter or enhancer element can increase expression of an RGS10 mRNA at the transcriptional level. A promoter or enhancer element can increase expression of an RGS10 mRNA at the posttranscriptional level. For example, at the transcriptional level, a promoter or enhancer element can increase expression by recruiting transcription factors, and/or RNA polymerase, increasing initiation of transcription or recruiting DNA and/or histone modifications that increase the level of transcription of RGS10. Such increase in expression can be detected by measuring an increase in the amount of RNA that is representative of the RGS10 transgene. At the posttranscriptional level, a regulatory element can increase expression by increasing the amount of protein that is translated into protein. This can be achieved through various mechanisms, for example, by increasing the stability of the mRNA or increasing recruitment and assembly of proteins required for translation. Such increase of expression can be detected by measuring the amount of protein expressed that is representative of the RGS10 transgene. The amount of protein produced can be measured directly, for example by an enzyme linked immunosorbent assay (ELISA) and western blotting, or indirectly, for example, by a functional assay. In some embodiments, the amount of protein produced can be measured by western blot. A protein commonly measured in a functional assay is luciferase. In some embodiments, the promoter or enhancer element described herein increases expression of RGS10 protein by at least 1.3, 1.4, 1.5, 1.6, 1.7, 1.8, 1.9, 2, 2.5, 3, 3.5, 4, 5, 6, 7, 8, 9, or 10 fold, or more than 3 fold, or more than 5 fold, or more than 10 fold, as compared to expression of RGS10 protein without the promoter or enhancer element.

Expression of mRNAs and proteins can be measured using any technique known in the art. In some instances, the relative and higher expression of a RGS10 mRNA when controlled by a promoter or enhancer element of the disclosure is measured using RNA quantification/sequencing techniques, such as quantitative PCR, northern blotting, or next generation sequencing. The same methods can also be used to measure a decrease in expression in RGS10.

In some instances, changes in expression can be measured by the concentration of the protein produced/expressed from the transgene/gene of interest before or after an event, such as an administrative of a composition described herein. The concentration of the protein can be measured by any method known in the art. Non-limiting examples of methods for measuring protein expression include, but not limited to, ELISA, radioimmunoassay, electrochemiluminescence immunoassays, western blotting, or high performance liquid chromatography. See, for examples, Noble J E, Quantification of protein concentration using UV absorbance and Coomassie dyes, Methods Enzymol. 2014;536:17-26; Kurien B T, Scofield R H. A brief review of other notable protein detection methods on acrylamide gels, Methods Mol Biol. (2012) 869:617-20; and Daniel M. Bollag, Michael D. Rozycki and Stuart J. Edelstein, Protein Methods, 2 ed., Wiley Publishers (1996). Protein expression can be measured in vitro, ex vivo, or in vivo.

In some examples, a promoter or enhancer element of this disclosure results in expression of an operably linked RGS10 nucleic acid at a level of at least 0.3, 0.4, 0.5, 1.0, 1.1, 1.2, 1.3, 1.4, 1.5, 1.6, 1.7, 1.8, 1.9, 2.0, 2.5, 3, 3.5, or 4.0 IU/ml in a relevant cell type as measured by western blotting or ELISA. In an in vivo example, a promoter or enhancer element of the disclosure can result in expression of an operably linked transcriptional expression of RGS10 or a decrease in RGS10 expression at an overall level of at least 0.3, 0.4, 0.5, 1.0, 1.1, 1.2, 1.3, 1.4, 1.5, 1.6, 1.7, 1.8, 1.9, 2.0, 2.5, 3, 3.5, or 4.0 IU/ml in microglia or CNS neurons of a mouse or other organism, as measured by western blotting or ELISA.

When assessing the activity of a vector, the activity or expression can be represented as an activity or expression level per unit dose, or normalized to a dose of vector administered or delivered into a cell, a tissue, an organ, or a subject such as a mouse, rat, or human. In some cases, expression or activity of an RGS10 protein or mRNA is normalized to an amount of plasmid or DNA (e.g., μg/kg per mouse), or viral particles (e.g., normalized to an amount of genome copies/kg per mouse) used to allow comparison across different expression vectors or cassettes with or without a regulatory element. For example, when assessing a regulatory element's activity in a mouse, expression or activity assayed can be normalized to a dose of about 5, 6, 7, 8, 9, 10, 11, 12, 13, 14, 15, 16, 17, 18, 19, 20, or greater than 10 or 500 μg of expression vector, cassette, or plasmid per mouse. In some cases, the expression level or activity can be normalized to 10⁷, 10⁸, 10⁹, 10¹⁰, 10¹¹, 10¹², 10¹³, 10¹⁴, or 10¹⁵ gc/kg (genome copies per kilogram) of viral particles containing an expression vector or cassette as disclosed herein per mouse.

The promoter sequences and enhancer sequences of this disclosure can be combined together or combined with other regulatory elements. Such other regulatory elements include, for examples, a constitutive promoter, an inducible promoter, a repressor, an enhancer or a posttranscriptional stability element. In one case, a vector comprises a minCMV promoter, upstream of a linked nucleic acid (e.g., SEQ ID NO: 1-5). Examples of promoters contemplated herein include: minCMV promoter, super core promoter, iCumate, PGK promoter, TTR promoter, Proto 1 promoter, UCL-HLP promoter, CMVe enhancer/CMV promoter combination, AAT promoter, EF1 α promoter, EFS promoter, CBA promoter, mouse Iba1 promoter, rat Iba1 promoter, or human IBA1 promoter.

The combined promoter sequence and enhancer sequence can come from different species. In preferred examples, at least one part of a combined regulatory element is human derived. Non-human derived elements can be derived from mammalian, viral, or synthetic sequences.

In some cases, a promoter sequence and enhancer sequence comprise a high expressing promoter or a sequence that increases mRNA stability and protein expression. In some cases, one or more promoter sequences and enhancer sequences are combined with a human, a non-human, or a non-mammalian sequence, for example a hSyn1 promoter, CBA promoter, a CMV promoter, an EF1α promoter, a polyA signal (e.g., the SV40 polyA signal), or a post-transcriptional regulatory element such as woodchuck hepatitis virus post-transcriptional regulatory element (WPRE).

Any known technique can be used to deliver the promoter sequence and RGS10 nucleic acid sequence, or compositions comprising the promoter sequence and RGS10 sequence, to the cells of interest (or target cell or cell type) to confer or induce in vitro, in vivo, or ex vivo expression of the nucleic acid in a cell-type specific manner. Techniques contemplated herein for gene therapy of brain cells include via a viral vector (e.g., retroviral, adenoviral, adeno-associated (AAV), herpes simplex, and lentivirus), and non-viral systems, such as physical systems (naked DNA, DNA bombardment, electroporation, hydrodynamic, ultrasound, and acoustic transduction), and chemical system (cationic lipids, different cationic polymers, and lipid polymers).

One limitation of using some common viral delivery systems is the size limitation of the viral vectors. For example, in some cases, the adeno-associated viruses (AAVs) have a capacity of approximately 4.4 kb. Thus, in some cases, there may be an advantage for using shorter promoter sequences and enhancer sequences to allow for delivery of a larger transgene, or shorter promoter sequences and enhancer sequences that drive expression of a transgene, or improve transgene expression, in a cell type specific manner.

In some cases, a promoter sequence or an enhancer sequence of the disclosure is (i) one that drives high expression of a RGS10 protein in a cell-type of interest, (ii) includes a human derived sequence, and/or (iii) and wherein the entire cloned insert into the vector is smaller than 3.0 kb, 2.5 kb, or 2 kb.

Also contemplated herein are AAV vectors, which can be a circular or linear nucleic acid molecule. A vector can be an integrating or non-integrating vector, referring to the ability of the vector to integrate the AAV vector and/or RGS10 nucleic acid sequence into a genome of a cell. Either an integrating vector or a non-integrating vector can be used to deliver an AAV vector containing a promoter sequence operably linked to a nucleic acid encoding a RGS10 protein. Other examples of vectors include, but are not limited to, viral vectors such as adeno-associated viral vectors, retroviral vectors, lentiviral vectors, adenoviral vectors, and herpes viral vectors.

### Serotype

Several serotypes of AAV, have been engineered for the purposes of gene delivery. Some AAV serotypes are known to have tropism for particular tissues or cell types. Viruses used for various gene-therapy applications can be engineered to be replication-deficient or to have low toxicity and low pathogenicity in a subject or a host. Such virus-based vectors can be obtained by deleting all, or some, of the coding regions from the viral genome, and leaving intact those sequences (e.g., inverted terminal repeat sequences) that are necessary for functions such as packaging the vector genome into the virus capsid or the integration of vector nucleic acid (e.g., DNA) into the host chromatin. An expression cassette comprising a transgene, for example a nucleic acid sequence that encodes an RGS10 protein, can be cloned into a viral backbone such as a modified or engineered viral backbone lacking viral genes, and used in conjunction with additional vectors (e.g., packaging vectors), which can, for example, when co-transfected, produce recombinant viral vector particles. In some embodiments, the AAV vector comprises AAV9. In some embodiments, the AAV vector comprises AAV1. In some embodiments, the AAV vector comprises AAV2. In some embodiments, the AAV vector comprises AAV3. In some embodiments, the AAV vector comprises AAV4. In some embodiments, the AAV vector comprises AAV5. In some embodiments, the AAV vector comprises AAV6. In some embodiments, the AAV vector comprises AAV7. In some embodiments, the AAV vector comprises AAV8. In some embodiments, the AAV vector comprises AAVrh10. In some embodiments, the AAV vector comprises AAV11. In some embodiments, the AAV vector comprises AAV12. In some embodiments, the AAV vector comprises AAV2/1. In some embodiments, the AAV vector comprises AAV2/2. In some embodiments, the AAV vector comprises AAV2/5. In some embodiments, the AAV vector comprises AAV2/6. In some embodiments, the AAV vector comprises AAV2/7. In some embodiments, the AAV vector comprises AAV2/8. In some embodiments, the AAV vector comprises AAV2/9. In some embodiments, the AAV vector comprises AAV2/rh10. In some embodiments, the AAV vector comprises AAV2/AAV11. In some embodiments, the AAV vector comprises AAV2/AAV12. In some embodiments, the AAV vector comprises a single stranded AAV (ssAAV). In some embodiments, the ssAAV comprises a single chain vector of serotype AAV2. In some embodiments, the ssAAV comprises a single chain vector of serotype AAV5. In some embodiments, the ssAAV comprises a single chain vector of serotype AAV9.

In some embodiments, the choice of AAV serotype determines in part the level of expression achievable of a target sequence of interest and an extent of spreading of expression of a target sequence of interest at a further distance from a site of administration. In some embodiments, the specific serotype of the AAV vector produces expression of a target sequence of interest (e.g., an RGS10 cDNA) that remains localized in close proximity to the site of administration of the AAV vector. In some embodiments, tightly localized expression of a target sequence of interest may be desirable to target delivery of a therapeutic protein (e.g., RGS10) expressed from a target sequence of interest to a CNS region occupying a small physical area. In some embodiments, the specific serotype of the AAV vector produces expression of a target sequence of interest (e.g., an RGS10 cDNA) that spreads to target regions of interest that are not in close proximity to the site of administration of the AAV vector. In some embodiments, widespread expression of a target sequence of interest may be desirable to target delivery of a therapeutic protein (e.g., RGS10) expressed from a target sequence of interest to a CNS region occupying a large physical area. In some embodiments, widespread expression of a target sequence of interest may be desirable to target delivery of a therapeutic protein (e.g., RGS10) expressed from a target sequence of interest to a CNS region in close proximity to a targeted site of injection and in more widespread, adjacent CNS regions. In some embodiments, the spreading of expression from the AAV vector produces a higher overall expression level of a target sequence of interest following injection of an equivalent volume and titer of AAV particles. In some embodiments, the AAV6 vector produces expression of a target sequence of interest (e.g., an RGS10 cDNA) that remains localized in close proximity to the site of administration of the AAV vector. In some embodiments, the AAV9 vector produces expression of a target sequence of interest (e.g., an RGS10 cDNA) that spreads to target regions of interest that are not in close proximity to the site of administration of the AAV vector. In some embodiments, the AAV9 vector produces a higher overall expression level of a target sequence of interest (e.g., an RGS10 cDNA) in areas of CNS surrounding a site of administration. In some embodiments, the AAV9 vector allows delivery of a higher overall expression level of a therapeutic protein of interest (e.g., an RGS10 protein) following expression from a target sequence of interest in areas of CNS adjacent to a site of administration.

In some embodiments, an AAV vector or an AAV viral particle, or virion, used to deliver an RGS10 transgene and a cell-specific promoter sequence into a cell, cell type, or tissue, in vivo or in vitro. In some embodiments, the AAV vector is replication-deficient. In some cases, an AAV vector is engineered so that it can replicate and generate virions only in the presence of helper factors.

In some embodiments, AAV tropism is determined by the capsid serotype. In some embodiment, an AAV vector comprising a nucleic acid sequence encoding a RGS10 protein or a functional fragment thereof further comprises an AAV capsid. In some embodiments, the AAV capsids comprises an AAV serotype AAV-2 capsid. In some embodiments, the AAV capsids comprises an AAV serotype AAV-5 capsid. In some embodiments, the AAV capsids comprises an AAV serotype AAV-9 capsid. In some embodiments, the AAV capsids comprises a pseudotyped AAV capsid. In some embodiments, the AAV-2 capsid allows for delivery of the AAV vector comprising a nucleic acid sequence encoding a RGS10 protein or a functional fragment thereof to microglia. In some embodiments, the AAV-2 capsid allows for delivery of the AAV vector comprising a nucleic acid sequence encoding a RGS10 protein or a functional fragment thereof to neurons. In some embodiments, the AAV-5 capsid allows for delivery of the AAV vector comprising a nucleic acid sequence encoding a RGS10 protein or a functional fragment thereof to microglia. In some embodiments, the AAV-5 capsid allows for delivery of the AAV vector comprising a nucleic acid sequence encoding a RGS10 protein or a functional fragment thereof to neurons. In some embodiments, the AAV-9 capsid allows for delivery of the AAV vector comprising a nucleic acid sequence encoding a RGS10 protein or a functional fragment thereof to microglia. In some embodiments, the AAV-9 capsid allows for delivery of the AAV vector comprising a nucleic acid sequence encoding a RGS10 protein or a functional fragment thereof to neurons. In some embodiments, the pseudotyped AAV capsid allows for delivery of the AAV vector comprising a nucleic acid sequence encoding a RGS10 protein or a functional fragment thereof to microglia. In some embodiments, the pseudotyped AAV capsid allows for delivery of the AAV vector comprising a nucleic acid sequence encoding a RGS10 protein or a functional fragment thereof to neurons. In some embodiments, the AAV vector comprising a capsid is formulated to be delivered to an CNS system of a subject.

### Microglial Cells

Compositions described herein, in some embodiments, may be used to target microglial cells for delivery of a therapeutic agent. In some embodiments, and AAV vector capable of expressing RGS10 protein or a functional fragment thereof is administered to the brain. In some embodiments, the AAV vector is administered to glial cells. In some embodiments, the glial cells comprise microglial cells. In some embodiments, the composition comprises a AAV serotype yielding high tropism toward the brain. In some embodiments, the high tropism toward the brain comprises high tropism to neurons, or microglia, or both neurons and microglia. In some embodiments, the high tropism toward microglia yields efficient delivery of AAV vector contents to microglia. In some embodiments, the efficient delivery of AAV vector contents to microglia produces modulated expression of RGS10 transgene in a plurality of microglial cells. In some embodiments, the efficient delivery of AAV vector contents to microglia produces increased expression of RGS10 transgene in a plurality of microglial cells. In some embodiments, the efficient delivery of AAV vector contents to microglia produces sustained expression of RGS10 transgene in a plurality of microglial cells. In some embodiments, the efficient delivery of AAV vector contents to microglia produces a restoration of expression of RGS10 protein levels in a plurality of microglial cells. In some embodiments, efficient delivery of AAV vector contents to microglia allows for initiation of expression of RGS10 transgene from a cell-specific promoter. In some embodiments, the cell-specific promoter initiates expression in cells of the brain. In some embodiments, the cell-specific promoter initiates expression in glial cells of the brain. In some embodiments, the cell-specific promoter modulates expression in microglia. In some embodiments, the cell-specific promoter initiates expression in microglia. In some embodiments, the cell-specific promoter maintains expression in microglia. In some embodiments, the cell-specific promoter increases expression in microglia. In some embodiments, the cell-specific promoter restores expression in microglia. In some embodiments, compositions used to increase expression of RGS10 in microglia can enhance neuron protection. In some embodiments, compositions used to increase expression of RGS10 in microglia modulate neuronal activity. In some embodiments, the modulation of neuronal activity comprises an increase in neuronal LTP. In some embodiments, the modulation of neuronal activity comprises a modulation in synaptic plasticity. In some embodiments, compositions used to increase expression of RGS10 in microglia increase phagocytosis of extracellular α-Syn aggregates. In some embodiments, compositions used to increase expression of RGS10 in microglia increase clearance of α-Syn aggregates. In some embodiments, compositions used to increase expression of RGS10 in microglia decrease quantities of brain α-Syn. In some embodiments, compositions used to increase expression of RGS10 in microglia decrease quantities of extracellular α-Syn aggregates. In some embodiments, compositions used to increase expression of RGS10 in microglia decrease an overproduction of a proinflammatory cytokine produced in response to an inflammatory stimulus. In some embodiments, compositions used to increase expression of RGS10 in microglia decrease an overproduction of a proinflammatory cytokine produced in response to a metabolic stress. In some embodiments, compositions used to increase expression of RGS10 in microglia decrease an overproduction of a proinflammatory cytokine produced in response to an inflammatory stimulus and a metabolic stress. In some embodiments, the proinflammatory cytokine comprises IL-1, IL-2, IL-6, IL-12, IL-17, IL-18, IFN-γ, or TNF-α. In some embodiments, compositions used to increase expression of RGS10 in microglia improves a response to antioxidant treatment. In some embodiments, compositions used to increase expression of RGS10 in microglia reduces oxidative stress. In some embodiments, compositions used to increase expression of RGS10 in microglia reduces neuronal inflammation. In some embodiments, compositions used to increase expression of RGS10 in microglia improves metabolic responses. In some embodiments, compositions used to increase expression of RGS10 in microglia improves metabolic responses in microglia and neurons. In some embodiments, compositions used to increase expression of RGS10 in microglia reduce ROS.

### Formulations

For administration of an injectable aqueous solution comprising an AAV vector, the solution may be suitably buffered, if necessary, and a liquid diluent first rendered isotonic with sufficient saline or glucose. For administration by injection, a suitable may include sterile aqueous solutions or dispersions. Dispersions may comprise glycerol, liquid polyethylene glycols, and mixtures thereof and in oils. Under ordinary conditions of storage and use, these preparations contain a preservative to prevent the growth of microorganisms. In some embodiments, AAV compositions are formulated to reduce aggregation of AAV particles in the composition. Methods for reducing aggregation of AAV include, for example, addition of surfactants, pH adjustment, salt concentration adjustment. Proper fluidity may be maintained, for example, by the use of a coating, such as lecithin or guar gum, by the maintenance of the required particle size in the case of dispersion and by the use of surfactants. In some embodiments, the AAV vector is formulated for direct administration to a central nervous system (CNS) of a subject. In some embodiments, the AAV vector is formulated for direct administration to the microglia of a subject.

A composition disclosed herein can be formulated in an artificial cerebrospinal fluid (CSF) formulation. The CSF formulation can comprise, for example, sodium phosphate, sodium chloride, potassium chloride, magnesium chloride, and calcium chloride. Disclosed herein is a composition comprising one or more pharmaceutically acceptable excipients, wherein the one or more pharmaceutically acceptable excipients comprise sodium phosphate, sodium chloride, potassium chloride, magnesium chloride, and calcium chloride. Disclosed herein is a composition, wherein the composition comprises 1 mM sodium phosphate, 148 sodium chloride, 3 mM potassium chloride, 0.8 mM magnesium chloride, 1.4 mM calcium chloride, pH 7.2.

### Dosages

In some embodiments, a dosage of a therapeutic agent is administered to a subject in an amount comprises an effective therapeutic dose. Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In one embodiment, the desired dose is conveniently presented in a single dose or in divided doses administered simultaneously (or over a short period of time) or at appropriate intervals, for example as two, three, four or more sub-doses per day. Non-limiting examples of effective dosages for intravenous administration of the therapeutic agent include at a rate between about 0.01 to 100 pmol/kg body weight/min. In various embodiments, dosages are altered depending on a number of variables including, but not limited to, the activity of the therapeutic agent used, the disease or disorder to be treated, the mode of administration, the requirements of the individual subject, the severity of the disease or disorder being treated, and the judgment of the practitioner.

The dose of AAV virion required to achieve a particular therapeutic effect, expressed as the units of dose in genome copies/per kilogram of body weight (gc/kg), may vary based on several factors. These factors include, but are not limited to: the route administration, the level of RGS10 protein expression required to achieve a therapeutic effect, the stability of the RGS10 mRNA, the stability of the RGS10 protein produced by the AAV vector, and the specific disease or disorder being treated. One of skill in the art can readily determine a AAV virion dose range to treat a patient having a particular disease or disorder based on the aforementioned factors, and other factors known in the art. An effective amount of an AAV is an amount sufficient to target infection in a subject. In some embodiments, the target infection is in a desired tissue. In some embodiments, the desired tissue is the brain of the subject. In some embodiments, the desired tissue is a region of the brain of the subject. In some embodiments, the target infection is a desired cell type. In some embodiments, the desired cell type is microglia. In some embodiments, the desired cell type is a neuron. An effective amount of the AAV vector comprising a nucleic acid sequence encoding a RGS10 protein is generally in the range from about 1 μL to about 80 mL of solution containing from about 10⁹ to 10¹⁶ genome copies. In some embodiments, a dosage of AAV vector comprises between about 10⁹-10¹⁰ gc. In some embodiments, a dosage of AAV vector comprises between about 10¹⁰-10¹¹ gc. In some embodiments, a dosage of AAV vector comprises between about 10¹¹-10¹² gc. In some embodiments, a dosage of AAV vector comprises between about 10¹²-10¹³gc. In some embodiments, a dosage of AAV vector comprises between about 10¹³-10¹⁴ gc. In some embodiments, a dosage of AAV vector comprises between about 10¹⁴-10¹⁵ gc. In some embodiments, a dosage of AAV vector comprises between about 10¹⁵-10¹⁶ gc. In some embodiments, 10¹¹-10¹² gc is effective to target tissue to treat a synucleinopathy. In some embodiments, 10¹²-10¹³ gc is effective to target tissue to treat a synucleinopathy. In some embodiments, 10¹³-10¹⁴ gc is effective to target tissue to treat a synucleinopathy. In some embodiments, 10¹²-10¹³ gc is effective to target cells to treat a synucleinopathy. In some embodiments, 10¹³-10¹⁴ gc is effective to target cells to treat a synucleinopathy.

### Methods

Provided herein are methods for enhancing neuron protection by administrating an AAV vector capable of modulating RGS10 expression in the brain. Also provided herein are methods for treating a disease or disorder by administrating an AAV vector capable of modulating RGS10 expression in the brain. In some embodiments, the disease or disorder is neurodegenerative.

Provided herein are methods for enhancing neuron protection comprising administering an AAV vector comprising a nucleic acid sequence encoding a RGS10 protein or a functional fragment thereof to a subject in need thereof. In some embodiments, enhancing neuron protection comprises slowing a rate of excessive neuronal loss. In some embodiments, enhancing neuron protection comprises converting a rate of excessive neuronal loss in a subject to a rate that is comparable for an age-matched reference subject. In some embodiments, enhancing neuron protection further comprises determining a neuron activity in the subject before and/or after the administrating. In some embodiments, neuron activity is determined by measuring LTP. In some embodiments, neuron activity is determined by measuring fEPSP in neurons. In some embodiments, neuron activity is determined by measuring fEPSP in hippocampal neurons. In some embodiments, neuron activity is determined by MRI. In some embodiments, neuron activity is determined by measuring electroencephalography (EEG) recordings. In some embodiments, LTP is assessed by examining human visual evoked responses using EEG recordings. In some embodiments, neuron activity is determined by Transcranial magnetic stimulation (TMS) to produce motor evoked potential (MEP).

In some embodiments, the quantitative measurement of neuron activity comprises two or more measurements in the subject at different points in time. In some embodiments, neuron activity is determined before and after the administering of a herein-described AAV vector. In some embodiments, neuron activity is determined about 1, 2, 3, 4, 5, 6, 7, 8, 9, 10, or 11 or 12 months after the administering of a herein-described AAV vector. In some embodiments, neuron activity is determined at least about 1, 2, 3, 4, 5, 6, 7, 8, 9, 10, or 11 or 12 months after the administering of a herein-described AAV vector. In some embodiments, neuron activity is determined at most about 1, 2, 3, 4, 5, 6, 7, 8, 9, 10, or 11 or 12 months after the administering of a herein-described AAV vector.

In some embodiments, methods for enhancing neuron protection comprising administering an AAV vector comprising a nucleic acid sequence encoding a RGS10 protein or a functional fragment thereof to a subject in need thereof comprise an improvement in microglial regulation of brain homeostasis. In some embodiments, the improvement in microglial regulation of brain homeostasis comprises a reduction in neuroinflammation. In some embodiments, the improvement in microglial regulation of brain homeostasis comprises a reduction in production of proinflammatory cytokines. In some embodiments, the improvement in microglial regulation of brain homeostasis comprises a reduction in secretion of proinflammatory cytokines. In some embodiments, the proinflammatory cytokine comprises IL-1, IL-2, IL-6, IL-12, IL-17, IL-18, IFN-γ, or TNF-α. In some embodiments, the improvement in microglial regulation of brain homeostasis comprises an improvement in a response to an antioxidant. In some embodiments, the improvement in microglial regulation of brain homeostasis comprises a reduction in oxidative stress. In some embodiments, the improvement in microglial regulation of brain homeostasis comprises a reduction in metabolic stress. In some embodiments, the improvement in microglial regulation of brain homeostasis comprises an increase in production of a neuroprotective agent. In some embodiments, the improvement in microglial regulation of brain homeostasis comprises an increase in secretion of a neuroprotective agent. In some embodiments, the improvement in microglial regulation of brain homeostasis comprises an increase in microglial ROS scavenging. In some embodiments, the improvement in microglial regulation of brain homeostasis comprises an increase microglial uptake of α-syn aggregates. In some embodiments, the improvement in microglial regulation of brain homeostasis comprises an increase microglial clearance of α-syn aggregates. In some embodiments, the improvement in microglial regulation of brain homeostasis comprises a decrease in brain α-syn levels.

Provided herein are methods for enhancing neuron protection comprising administering an AAV vector comprising a nucleic acid sequence encoding a RGS10 protein or a functional fragment thereof to a subject in need thereof. In some embodiments, the subject has a disease or disorder. In some embodiments, the subject has been diagnosed with neurodegeneration. In some embodiments, the subject is at risk of developing neurodegeneration. In some embodiments, the disease or disorder is a synucleinopathy. In some embodiments, the synucleinopathy further comprises Type 2 diabetes. In some embodiments, the synucleinopathy further comprises diabetic neuropathy. In some embodiments, the synucleinopathy further comprises obesity. In some embodiments, the synucleinopathy further Type 2 diabetes and diabetic neuropathy. In some embodiments, the synucleinopathy further Type 2 diabetes and obesity. In some embodiments, the synucleinopathy further diabetic neuropathy and obesity. In some embodiments, the synucleinopathy further Type 2 diabetes, diabetic neuropathy, and obesity. In some embodiments, the synucleinopathy comprises Parkinson's Disease. In some embodiments, the synucleinopathy comprises Dementia with Lewy Body. In some embodiments, the synucleinopathy comprises Essential Tremor. In some embodiments, the synucleinopathy comprises Multiple System Atrophy. In some embodiments, the synucleinopathy comprises Frontotemporal dementia. In some embodiments, the synucleinopathy comprises Alzheimer's Disease. In some embodiments, the synucleinopathy comprises AD/ALB.

Provided herein are methods for treating a disease or disorder comprising administering an AAV vector comprising a nucleic acid sequence encoding a RGS10 protein or a functional fragment thereof to a subject in need thereof. In some embodiments, the administering increases expression of RGS10 protein or a functional fragment thereof in the brain. In some embodiments, the administering increases expression of RGS10 protein or a functional fragment thereof in a certain region of the brain. In some embodiments, the administering increases expression of RGS10 protein or a functional fragment thereof in a plurality of neurons. In some embodiments, the administering increases expression of RGS10 protein or a functional fragment thereof in a plurality of microglia. In some embodiments, the administering increases expression of RGS10 protein or a functional fragment thereof in a plurality of neurons and microglia.

In some embodiments, methods comprising an increase in expression of RGS10 protein or a functional fragment thereof modulate an immune response in the brain of the subject. In some embodiments, the increased expression of RGS10 protein or a functional fragment thereof decreases an amount of production of a pro-inflammatory cytokine. In some embodiments, the increased expression of RGS10 protein or a functional fragment thereof decreases an amount of secretion of a pro-inflammatory cytokine. In some embodiments, the pro-inflammatory cytokine comprises IL-1. In some embodiments, the pro-inflammatory cytokine comprises IL-2. In some embodiments, the pro-inflammatory cytokine comprises IL-6. In some embodiments, the pro-inflammatory cytokine comprises IL-12. In some embodiments, the pro-inflammatory cytokine comprises IL-17. In some embodiments, the pro-inflammatory cytokine comprises IL-18. In some embodiments, the pro-inflammatory cytokine comprises IFN-γ. In some embodiments, the pro-inflammatory cytokine comprises TNF-α.

In some embodiments, methods comprising an increase in expression of RGS10 protein or a functional fragment thereof to treat a disease or disorder in a subject comprise enhanced neuron protection. In some embodiments, the increased expression of RGS10 protein or a functional fragment thereof increases an amount of production of a neuroprotective substance. In some embodiments, the increased expression of RGS10 protein or a functional fragment thereof increases an amount of secretion of a neuroprotective substance. In some embodiments, the neuroprotective substance is producing in microglia. In some embodiments, the neuroprotective substance is secreted by microglia. In some embodiments, the neuroprotective substance is assayed in microglia.

In some embodiments, the disease or disorder comprises a neurodegeneration. In some embodiments, the disease or disorder is a synucleinopathy. In some embodiments, the disease or disorder comprises a metabolic condition. In some embodiments, the disease or disorder further comprises Type 2 diabetes. In some embodiments, the disease or disorder further comprises diabetic neuropathy. In some embodiments, the disease or disorder further comprises obesity. In some embodiments, the disease or disorder further comprises Type 2 diabetes and diabetic neuropathy. In some embodiments, the disease or disorder further comprises Type 2 diabetes and obesity. In some embodiments, the disease or disorder further comprises diabetic neuropathy and obesity. In some embodiments, the disease or disorder further comprises Type 2 diabetes, diabetic neuropathy, and obesity. In some embodiments, the synucleinopathy comprises Parkinson's Disease. In some embodiments, the synucleinopathy comprises Dementia with Lewy Body. In some embodiments, the synucleinopathy comprises Essential Tremor. In some embodiments, the synucleinopathy comprises Multiple System Atrophy. In some embodiments, the synucleinopathy comprises Pure Autonomic Failure. In some embodiments, the synucleinopathy comprises AD/ALB. In some embodiments, the subject has been diagnosed with a disease or disorder. In some embodiments, the subject has not been diagnosed with a disease or disorder.

Provided herein are methods of increasing an RGS10 protein level in a brain of a subject in need thereof, the method comprising administering an AAV vector comprising a nucleic acid sequence encoding an RGS10 protein or a functional fragment thereof to the subject, wherein the subject has been diagnosed with or is at risk for developing a neurodegenerative disease. In some embodiments, the administering the AAV vector comprises administering to the brain. In some embodiments, the administering the AAV vector comprises administering to neurons. In some embodiments, the administering the AAV vector comprises administering to glia. In some embodiments, the glia comprise microglia. In some embodiments, the subject has been diagnosed with or is at risk for developing an amyloid pathology. In some embodiments, the amyloid pathology comprises a synuclein pathology. In some embodiments, the amyloid pathology comprises a β amyloid peptide (Aβ) pathology. In some embodiments, the amyloid pathology comprises a tau pathology. In some embodiments, the amyloid pathology comprises a TDP-43 pathology. In some embodiments, the subject has been diagnosed with or is at risk for developing a neurodegenerative disease. In some embodiments, the neurodegenerative disease is characterized by an amyloid aggregate pathology. In some embodiments, the amyloid aggregate pathology is α-synuclein pathology. In some embodiments, the amyloid aggregate pathology is β amyloid peptide (Aβ) pathology. In some embodiments, the amyloid aggregate pathology is tau pathology. In some embodiments, the amyloid aggregate pathology is FUS pathology. In some embodiments, the amyloid aggregate pathology is TDP-43 pathology. In some embodiments, the amyloid aggregate pathology comprises α-synuclein pathology, β amyloid peptide (Aβ) pathology, tau pathology, FUS pathology, or TDP-43 pathology, or a combination thereof. In some embodiments, the disease or disorder further comprises Type 2 diabetes, diabetic neuropathy, and obesity. In some embodiments, the neurodegenerative disease is Parkinson's Disease. In some embodiments, the neurodegenerative disease is Dementia with Lewy Body. In some embodiments, the neurodegenerative disease is Essential Tremor. In some embodiments, the neurodegenerative disease is Multiple System Atrophy. In some embodiments, the neurodegenerative disease is Pure Autonomic Failure. In some embodiments, the neurodegenerative disease is AD/ALB.

In some embodiments, the administering the AAV vector to microglia comprises an increase in microglial protein scavenging activity. In some embodiments, the increase in microglial protein scavenging activity comprises an increase in α-syn aggregate scavenging. In some embodiments, the increase in microglial protein scavenging activity comprises an increase in α-syn aggregate phagocytosis. In some embodiments, the increase in microglial protein scavenging activity comprises an increase in α-syn aggregate clearance. In some embodiments, the α-syn aggregates comprises intracellular α-syn aggregates. In some embodiments, the α-syn aggregates comprises extracellular α-syn aggregates. In some embodiments, the α-syn aggregates comprises intracellular and extracellular α-syn aggregates. In some embodiments, the increase in microglial protein scavenging activity comprises a decrease in brain α-syn levels. In some embodiments, the decrease in brain α-syn levels comprises of reduction of at least 1%, 2%, 3%, 4%, 5%, 6%, 7%, 8%, 9%, 10%, 11%, 12%, 13%, 14%, 15%, 16%, 17%, 18%, 19%, 20%, 22%, 25%, 27%, 30%, 35%, 40%, 45%, 50%, 60%, 70%, 80%, or 90% of α-syn levels. In some embodiments, administering decreases a level of α-syn in a plurality of neurons by at least 5%. In some embodiments, administering decreases a level of α-syn aggregates in a plurality of neurons by at least 5%. In some embodiments, the level of α-syn aggregates is measured quantitatively using an antibody that detects phosphorylated α-syn.

In some embodiments, administering the AAV vector comprising a nucleic acid sequence encoding a RGS10 protein or a functional fragment thereof to the subject comprises delivering the nucleic acid sequence to a plurality of neurons. In some embodiments, administering the AAV vector comprising a nucleic acid sequence encoding a RGS10 protein or a functional fragment thereof to the subject comprises delivering the nucleic acid sequence to a plurality of microglia. In some embodiments, administering the AAV vector comprising a nucleic acid sequence encoding a RGS10 protein or a functional fragment thereof to the subject comprises delivering the nucleic acid sequence to a plurality of astrocytes. In some embodiments, administering the AAV vector comprising a nucleic acid sequence encoding a RGS10 protein or a functional fragment thereof to the subject comprises delivering the nucleic acid sequence to a plurality of neurons and microglia. In some embodiments, the administering increases a level of RGS10 protein in a plurality of neurons by at least 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, 100%, 125%, 150%, 175%, 200%, 250%, 300%, 350%, 400%, 500%, 600%, 700%, 800%, 1000%, 1200%, 1500%, 2000%, 3000%, 4000% or 5000%. In some embodiments, the administering increases a level of RGS10 protein in a plurality of microglia by at least 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, 100%, 125%, 150%, 175%, 200%, 250%, 300%, 350%, 400%, 500%, 600%, 700%, 800%, 1000%, 1200%, 1500%, 2000%, 3000%, 4000% or 5000%. In some embodiments, the administering increases a level of RGS10 protein in a plurality of microglia and neurons by at least 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, 100%, 125%, 150%, 175%, 200%, 250%, 300%, 350%, 400%, 500%, 600%, 700%, 800%, 1000%, 1200%, 1500%, 2000%, 3000%, 4000% or 5000%. In some embodiments, the administering increases a level of RGS10 protein in a plurality of neurons by at least 5%. In some embodiments, the administering increases a level of RGS10 protein in a plurality of microglia by at least 5%. In some embodiments, the administering increases a level of RGS10 protein in a plurality of neurons and microglia by at least 5%. In some embodiments, the increase in a level of RGS10 protein in a plurality of microglia is increased to an extent that compensates for an age-related decrease in brain RGS10 protein levels. In some embodiments, the increase in a level of RGS10 protein in a plurality of neurons is increased to an extent that compensates for an age-related decrease in brain RGS10 protein levels. In some embodiments, the increase in a level of RGS10 protein in a plurality of microglia and neurons is increased to an extent that compensates for an age-related decrease in brain RGS10 protein levels.

In some embodiments, the administering an AAV vector comprising a nucleic acid sequence encoding a RGS10 protein or a functional fragment thereof to the subject results in a change in synaptic plasticity. In some embodiments, the administering an AAV vector comprising a nucleic acid sequence encoding a RGS10 protein or a functional fragment thereof to the subject results in an improvement in learning. In some embodiments, the administering an AAV vector comprising a nucleic acid sequence encoding a RGS10 protein or a functional fragment thereof to the subject results in an improvement in memory. In some embodiments, the administering an AAV vector comprising a nucleic acid sequence encoding a RGS10 protein or a functional fragment thereof to the subject results in an increase in LTP is measured in the hippocampus. In some embodiments, the increase in LTP is not abrogated by metabolic stress. In some embodiments, the increase in LTP is not abrogated by inflammatory stress. In some embodiments, the increase in LTP is not abrogated by oxidative stress. In some embodiments, the increase in LTP is not abrogated by metabolic stress or inflammatory stress.

In some embodiments, administration that increase the protein level of RGS10 in a plurality of microglia results in an improvement of α-syn aggregate pathology in a preformed fibrils (PFFs) model. In some embodiments, administration that increase the protein level of RGS10 in a plurality of neurons results in an improvement of α-syn aggregate pathology in a preformed fibrils (PFFs) model. In some embodiments, administration that increase the protein level of RGS10 in a plurality of neurons and microglia results in an improvement of α-syn aggregate pathology in a preformed fibrils (PFFs) model.

In some embodiments, the administering of the AAV vector increases an RGS10 mRNA expression level by at least 5%. In some embodiments, the administering of the AAV vector to a plurality of neurons increases an RGS10 mRNA expression level by at least 5% after. In some embodiments, the administering of the AAV vector to a plurality of neurons and microglia increases an RGS10 mRNA expression level by at least 5%. In some embodiments, the increase in RGS10 mRNA expression is maintained in a plurality of neurons, in a plurality of microglia cells, or in a plurality of neurons and microglia cells for at least about 1, 2, 3, 4, 5, 6, 7, 8, 9, 10, 11, 12, 13, 14, 15, or 16 weeks following the administering. In some embodiments, the increase in RGS10 mRNA expression is maintained in a plurality of neurons, in a plurality of microglia cells, or in a plurality of neurons and microglia cells for at least about 4 weeks following the administering. In some embodiments, a sufficient level of RGS10 mRNA expression is maintained to provide a therapeutically effective amount of RGS10 protein to a plurality of neurons, a plurality of microglia cells, or a plurality of neurons and microglia cells in the subject.

In some embodiments, a level of RGS10 protein in microglia of the subject is assayed at a time point after administration of the AAV vector. In some embodiments, the time point after administration is from 1-48 hours. In some embodiments, the time point after administration is 1 to 180 days. In some embodiments, the time point after administration is 1 to 30, 1 to 60, 1 to 90, or 1 to 120 days. In some embodiments, the time point after administration is to 12, 1 to 48, 1 to 72, or 1 to 120 hours. In some embodiments, the time point after administration is about 1, 2, 3, 4, 5, 6, 7, 8, 9, 10, 11, 12, 24, 36, or 48 hours. In some embodiments, the time point after administration is at least about 1, 2, 3, 4, 5, 6, 7, 8, 9, 10, 11, 12, 24, 36, or 48 hours. In some embodiments, the time point after administration is at most about 1, 2, 3, 4, 5, 6, 7, 8, 9, 10, 11, 12, 24, 36, or 48 hours. In some embodiments, the time point after administration is about 1, 2, 3, 4, 5, 6, 7, 8, 9, 10, 11, 12, 24, 36, or 48 days. In some embodiments, the time point after administration is at least about 1, 2, 3, 4, 5, 6, 7, 8, 9, 10, 11, 12, 24, 36, or 48 days. In some embodiments, the time point after administration is at most about 1, 2, 3, 4, 5, 6, 7, 8, 9, 10, 11, 12, 24, 36, or 48 days. In some embodiments, the time point after administration is about 10, 20, 30, 40, 50, 60, 70, 80, 100, 150, 200, 250, 300, 360, or 730 days. In some embodiments, the time point after administration is at least about 10, 20, 30, 40, 50, 60, 70, 80, 100, 150, 200, 250, 300, 360, or 730 days. In some embodiments, the time point after administration is at most about 10, 20, 30, 40, 50, 60, 70, 80, 100, 150, 200, 250, 300, 360, or 730 days.

In some embodiments, a level of RGS10 protein in neurons of the subject is assayed at a time point after administration of the AAV vector. In some embodiments, the time point after administration is from 1-48 hours. In some embodiments, the time point after administration is 1 to 180 days. In some embodiments, the time point after administration is 1 to 30, 1 to 60, 1 to 90, or 1 to 120 days. In some embodiments, the time point after administration is to 12, 1 to 48, 1 to 72, or 1 to 120 hours. In some embodiments, the time point after administration is about 1, 2, 3, 4, 5, 6, 7, 8, 9, 10, 11, 12, 24, 36, or 48 hours. In some embodiments, the time point after administration is at least about 1, 2, 3, 4, 5, 6, 7, 8, 9, 10, 11, 12, 24, 36, or 48 hours. In some embodiments, the time point after administration is at most about 1, 2, 3, 4, 5, 6, 7, 8, 9, 10, 11, 12, 24, 36, or 48 hours. In some embodiments, the time point after administration is about 1, 2, 3, 4, 5, 6, 7, 8, 9, 10, 11, 12, 24, 36, or 48 days. In some embodiments, the time point after administration is at least about 1, 2, 3, 4, 5, 6, 7, 8, 9, 10, 11, 12, 24, 36, or 48 days. In some embodiments, the time point after administration is at most about 1, 2, 3, 4, 5, 6, 7, 8, 9, 10, 11, 12, 24, 36, or 48 days. In some embodiments, the time point after administration is about 10, 20, 30, 40, 50, 60, 70, 80, 100, 150, 200, 250, 300, 360, or 730 days. In some embodiments, the time point after administration is at least about 10, 20, 30, 40, 50, 60, 70, 80, 100, 150, 200, 250, 300, 360, or 730 days. In some embodiments, the time point after administration is at most about 10, 20, 30, 40, 50, 60, 70, 80, 100, 150, 200, 250, 300, 360, or 730 days.

In some embodiments, at the time point after administration, a level of RGS10 protein in the microglia of the subject is increased by at least 5%. In some embodiments, at the time point after administration, a level of RGS10 protein in the microglia of the subject is increased by at least 10%. In some embodiments, at the time point after administration, a level of RGS10 protein in the microglia of the subject is increased by at least 20%. In some embodiments, at the time point after administration, a level of RGS10 protein in the microglia of the subject is increased by at least 30%. In some embodiments, at the time point after administration, a level of RGS10 protein in the microglia of the subject is increased by at least 50%. In some embodiments, at the time point after administration, a level of RGS10 protein in the microglia of the subject is increased by at least 100%. In some embodiments, the increase in RGS10 protein is maintained in a plurality of microglia cells for at least about 1, 2, 3, 4, 5, 6, 7, 8, 9, 10, 11, 12, 13, 14, 15, or 16 weeks following the administration. In some embodiments, the increase in RGS10 protein is maintained in a plurality of microglia cells for at least about 4 weeks following the administration. In some embodiments, a sufficient level of RGS10 mRNA expression is maintained to provide a therapeutically effective amount of RGS10 protein to a plurality of microglia cells in the subject.

In some embodiments, at the time point after administration, a level of RGS10 protein in a plurality of neurons of the subject is increased by at least 5%. In some embodiments, at the time point after administration, a level of RGS10 protein in a plurality of neurons of the subject is increased by at least 10%. In some embodiments, at the time point after administration, a level of RGS10 protein in a plurality of neurons of the subject is increased by at least 20%. In some embodiments, at the time point after administration, a level of RGS10 protein in a plurality of neurons of the subject is increased by at least 30%. In some embodiments, at the time point after administration, a level of RGS10 protein in a plurality of neurons of the subject is increased by at least 50%. In some embodiments, at the time point after administration, a level of RGS10 protein in a plurality of neurons of the subject is increased by at least 100%. In some embodiments, the increase in RGS10 protein is maintained in a plurality of neurons for at least about 1, 2, 3, 4, 5, 6, 7, 8, 9, 10, 11, 12, 13, 14, 15, or 16 weeks following the administration. In some embodiments, the increase in RGS10 protein is maintained in a plurality of neurons for at least about 4 weeks following the administration. In some embodiments, a sufficient level of RGS10 mRNA expression is maintained to provide a therapeutically effective amount of RGS10 protein to a plurality of neurons in the subject.

In some embodiments, at the time point after administration, a level of RGS10 protein in a plurality of neurons and in a plurality of microglia cells of the subject is increased by at least 5%. In some embodiments, at the time point after administration, a level of RGS10 protein in a plurality of neurons and in a plurality of microglia cells of the subject is increased by at least 10%. In some embodiments, at the time point after administration, a level of RGS10 protein in a plurality of neurons and in a plurality of microglia cells of the subject is increased by at least 20%. In some embodiments, at the time point after administration, a level of RGS10 protein in a plurality of neurons and in a plurality of microglia cells of the subject is increased by at least 30%. In some embodiments, at the time point after administration, a level of RGS10 protein in a plurality of neurons and in a plurality of microglia cells of the subject is increased by at least 50%. In some embodiments, at the time point after administration, a level of RGS10 protein in a plurality of neurons and in a plurality of microglia cells of the subject is increased by at least 100%. In some embodiments, the increase in RGS10 protein is maintained in a plurality of neurons and in a plurality of microglia cells for at least about 1, 2, 3, 4, 5, 6, 7, 8, 9, 10, 11, 12, 13, 14, 15, or 16 weeks following the administration. In some embodiments, the increase in RGS10 protein is maintained in in a plurality of neurons and in a plurality of microglia cells for at least about 4 weeks following the administration. In some embodiments, a sufficient level of RGS10 mRNA expression is maintained to provide a therapeutically effective amount of RGS10 protein to a plurality of neurons and to a plurality of microglia cells in the subject.

In some embodiments, the administering of the AAV vector to a plurality of microglia increases microglial responsiveness to metabolic aspects. In some embodiments, the increase in microglial responsiveness to metabolic aspects comprises an increase in microglia insulin sensitivity. In some embodiments, the increase in microglial responsiveness to metabolic aspects comprises an increase in neuronal insulin sensitivity. In some embodiments, the increase in microglial responsiveness to metabolic aspects comprises a decreased effect of metabolic stress on microglia. In some embodiments, the increase in microglial responsiveness to metabolic aspects comprises a decreased effect of metabolic stress on neurons. In some embodiments, the administering of the AAV vector to a subject results in an improvement in glucose tolerance test of the subject.

In some embodiments, the administering of the AAV vector to the brain reduces oxidative stress. In some embodiments, the administering of the AAV vector to a plurality of microglia reduces oxidative stress. In some embodiments, the administering of the AAV vector to a plurality neurons reduces of oxidative stress. In some embodiments, the administering of the AAV vector to a plurality of microglia and neurons reduces oxidative stress. In some embodiments, the reduction in oxidative stress is within the plurality of microglia. In some embodiments, the reduction in oxidative stress is within the plurality of neurons. In some embodiments, the reduction in oxidative stress is within the plurality microglia and neurons. In some embodiments, administering reduces the amount of intracellular ROS in the plurality of microglia. In some embodiments, administering reduces the amount of intracellular RNS in the plurality of microglia. In some embodiments, administering reduces the amount of intracellular ROS and RNS in the plurality of microglia.

In some embodiments, the administering of the AAV vector to the brain results in a reduction in neuroinflammation. In some embodiments, the administering the AAV vector to microglia results in a reduction in neuroinflammation. In some embodiments, the administering the AAV vector to neurons results in a reduction in neuroinflammation. In some embodiments, the administering the AAV vector to microglia and neurons results in a reduction in neuroinflammation. In some embodiments, the reduction in neuroinflammation comprises a reduction in production of a proinflammatory cytokine. In some embodiments, the reduction in neuroinflammation comprises a reduction in secretion of a proinflammatory cytokine. In some embodiments, the pro-inflammatory cytokine comprises IL-1. In some embodiments, the pro-inflammatory cytokine comprises IL-2. In some embodiments, the pro-inflammatory cytokine comprises IL-6. In some embodiments, the pro-inflammatory cytokine comprises IL-12. In some embodiments, the pro-inflammatory cytokine comprises IL-17. In some embodiments, the pro-inflammatory cytokine comprises IL-18. In some embodiments, the pro-inflammatory cytokine comprises IFN-γ. In some embodiments, the pro-inflammatory cytokine comprises TNF-α.

In some embodiments, the administering of the AAV vector results in an increase in RGS10 protein levels in the brain. In some embodiments, the administering of the AAV vector results in an increase in RGS10 mRNA levels in the brain. In some embodiments, the increase comprises increase in neurons, microglia, or both neurons and microglia. In some embodiments, the increase in RGS10 mRNA levels is assayed using standard techniques such as quantitative RT-PCR, northern blotting, or next generation sequencing. In some embodiments, the increase in RGS10 protein levels comprises at least a 1%, 2%, 3%, 4%, 5%, 6%, 7%, 8%, 9%, 10%, 11%, 12%, 13%, 14%, 15%, 16%, 17%, 18%, 19%, 20%, 22%, 25%, 27%, 30%, 35%, 40%, 45%, 50%, 60%, 70%, 80%, or 90% increase in RGS10 protein. In some embodiments, the increase in RGS10 mRNA levels comprises at least a 1%, 2%, 3%, 4%, 5%, 6%, 7%, 8%, 9%, 10%, 11%, 12%, 13%, 14%, 15%, 16%, 17%, 18%, 19%, 20%, 22%, 25%, 27%, 30%, 35%, 40%, 45%, 50%, 60%, 70%, 80%, or 90% increase in RGS10 mRNA.

### Modes of Administration

The therapies for neurodegenerative conditions disclosed herein may be administered to a subject disclosed herein in certain doses, frequency, and by different routes or modes of administration according to various embodiments herein. In some embodiments, the therapy for a neurodegenerative condition comprises administration of a therapeutic agent. In some embodiments, methods disclosed herein comprise administering a therapeutic agent by injection. In some embodiments, the AAV vector is administered via peripheral injection. In some embodiments, the injection comprises intravenous delivery. In some embodiments, the injection comprises intravascular delivery. In some embodiments, the injection comprises intrathecal delivery. In some embodiments, the injection comprises intracisternal delivery. In some embodiments, the injection comprises intraspinal delivery. In some embodiments, the injection comprises intraventricular delivery. In some embodiments, the injection comprises stereotactic delivery. In some embodiments, the injection comprises subpial delivery. In some embodiments, the injection comprises intracerebroventricular delivery. In some embodiments, the AAV vector is administered via stereotaxic injection into the brain parenchyma. In some embodiments, the AAV vector is administered via stereotaxic injection into the spinal cord parenchyma.

In some embodiments, a AAV composition disclosed herein is administered by introduction into the central nervous system of the subject, for example, into the cerebrospinal fluid of the subject. In some embodiments, routes for local delivery closer to a site needing treatment are preferred over systemic routes. Routes, dosage, time points, and duration of administrating therapeutics may be adjusted. In some embodiments, administration of a therapeutic agent is prior to, or after, onset of either, or both, acute and chronic symptoms of the disease or disorder. In some embodiments, administration of a therapeutic agent is after onset of acute symptoms of the disease or disorder. In some embodiments, administration of a therapeutic agent is after onset of chronic symptoms of the disease or disorder.

An effective dose and dosage of a therapeutic agent to prevent or treat the disease or disorder disclosed herein is defined by an observed beneficial response related to the disease or disorder, or symptom of the disease or disorder. Beneficial response comprises preventing, alleviating, arresting, or curing the disease or disorder, or symptom of the disease or disorder. In some embodiments, the beneficial response may be measured by detecting a measurable improvement in the presence, level, or activity, of a biomarker associated with neurodegeneration. An “improvement,” as used herein refers to shift in the presence, level, or activity towards a presence, level, or activity, observed in normal individuals (e.g., individuals who do not suffer from the disease or disorder).

In some embodiments, the therapeutic agent is delivered to the brain by a means that risks minimal damage to brain tissue. In some embodiments, the therapeutic agent is delivered locally to a brain region that has been identified as exhibiting a pathology characteristic of the disease or disorder. In some embodiments, the therapeutic agent is delivered to the brain by a means that both risks minimal damage to brain tissue and is delivered locally to a brain region that has been identified as exhibiting a pathology characteristic of the disease or disorder.

In some embodiments, an AAV composition disclosed herein is administered by lateral cerebroventricular injection into the brain of a subject. The injection can be made, for example, through a burr hole made in the subject's skull. In some embodiments, an AAV composition disclosed herein is administered through a surgically inserted shunt into the cerebral ventricle of a subject. For example, the injection can be made into the lateral ventricles, which are larger, even though injection into the third and fourth smaller ventricles can also be made.

In some embodiments, a AAV composition disclosed herein is delivered to one or more surface or shallow tissues of the brain, cerebrum, or spinal cord. In some embodiments, the targeted surface or shallow tissues of the cerebrum are selected from pia mater tissues, cerebral cortical ribbon tissues, hippocampus, Virchow Robin (VR) space, blood vessels within the VR space, portions of the hypothalamus on the inferior surface of the brain, the optic nerves and tracts, the olfactory bulb and projections, and combinations thereof.

In some embodiments, a AAV composition disclosed herein is delivered to one or more deep tissues of the cerebrum or spinal cord. In some embodiments, the targeted surface or shallow tissues of the cerebrum or spinal cord are located 2 mm, 3 mm, 4 mm, 5 mm, 6 mm, 7 mm, 8 mm, 9 mm, or 10 mm below the surface of the cerebrum. In some embodiments, targeted deep tissues of the cerebrum include the cerebral cortical ribbon. In some embodiments, targeted deep tissues of the cerebrum include one or more of the diencephalon, the hypothalamus, thalamus, prethalamus, subthalamus, striatum, metencephalon, lentiform nuclei, the basal ganglia, caudate, putamen, amygdala, globus pallidus, and combinations thereof.

In some embodiments, a AAV composition disclosed herein is delivered to various cells in the brain including, but not limited to, neurons of the cerebral cortex, neurons of the striatum, hippocampal neurons, glial cells, perivascular cells, or meningeal cells.

In some embodiments, the AAV composition disclosed herein can be administered alone, or in conjunction with other agents, such as antihistamines (e.g., diphenhydramine) or immunosuppressants or other immunotherapeutic agents. A AAV composition described herein can be administered before, at the same time, or after an additional therapeutic agent. For example, the additional therapeutic agent can be mixed into a composition containing the therapeutic protein, and thereby administered together with the therapeutic agent. The agent can be administered separately (e.g., not admixed), but within a short time frame (e.g., within 24 hours) of administration of the therapeutic agent. Non-limiting examples of pharmaceutically active agents suitable for combination with compositions of the disclosure include anti-infectives (e.g., aminoglycosides, antiviral agents, antimicrobials, anti-cholinergics/anti-spasmotics, antidiabetic agents, antihypertensive agents, anti-neoplastics, cardiovascular agents, central nervous system agents, coagulation modifiers, hormones, immunologic agents, and immunosuppressive agents).

An AAV composition disclosed herein can be administered after the onset of symptoms of any condition disclosed herein. An AAV composition disclosed herein can demonstrate increased efficacy if administered at a late stage of disease, rather than at an early stage of disease.

In some embodiments, a AAV composition disclosed herein are administered at intervals greater than 30, 60, 90, 120, 150, 180, 210, 240, 270, 300, 330, 364, 729, or 1094 days.

### Kits for Use in a Method Disclosed Herein

Compositions disclosed herein can be packaged as a kit. In some embodiments, the present disclosure provides a kit comprising a compound disclosed herein, and written instructions on use of the kit in the treatment of a condition described herein.

A kit disclosed herein can comprise a AAV vector comprising an RGS10 transgene regulated by cell-specific promoter described herein for use in the treatment of a synucleinopathy, in a dose and form suitable for administration to a subject. In some embodiments, the kit can comprise a device for delivering the AAV intrathecally. In some embodiments, the kit can comprise a device for delivering the AAV intravascularly.

The kit disclosed herein can comprise instructions for the administration of a composition disclosed herein. In some embodiments, a kit disclosed herein comprises a catheter or other device for intrathecal administration of a composition disclosed herein. In some embodiments, the device comprises a catheter or other device for intravascular administration of the composition. For example, a kit disclosed herein can comprise a catheter preloaded with 10⁹-10¹⁰, 10¹⁰-10¹¹, 10¹¹-10¹², 10¹²-10¹³, 10¹³-10¹⁴, or 10¹⁴-10¹⁵ gc/kg of a therapeutic AAV vector comprising an RGS10 transgene, in a pharmaceutically acceptable formulation.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof. Furthermore, to the extent that the terms “including”, “includes”, “having”, “has”, “with”, or variants thereof are used in either the detailed description and/or the claims, such terms are intended to be inclusive in a manner similar to the term “comprising”.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” or “individual,” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. The term “patient” can refer to a subject. In some instances, a patient has received a diagnosis of a disease or disorder. In some instances, a patient has received a diagnosis of a disease or disorder and will undergo a treatment. In some instances, a patient has received a diagnosis of a disease or disorder and has begun a treatment. In some instances, a patient has received a diagnosis of a disease or disorder and has completed a treatment regimen.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The terms “microglia” and “microglial cells” refer to a type of glia cell found in the central nervous system. These cells can function as the resident macrophages of the CNS and form an aspect of the immune system operating in the CNS. The terms “microglia” and “microglial cells” are herein used interchangeably.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to an intervention regimen for obtaining beneficial or desired results in the recipient. In some cases, this involves gene therapy. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or disorder, delaying or eliminating the onset of symptoms of a disease or disorder, slowing, halting, or reversing the progression of a disease or disorder, or any combination thereof. For prophylactic benefit, a subject at risk of developing a particular disease, or to a subject reporting one or more of the physiological symptoms of a disease may undergo treatment, even though a diagnosis of this disease may not have been made.

The term “therapeutically effective amount” refers to the amount of a viral vector or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician. In some cases, therapeutically effective amount of the viral vector reduces the severity of symptoms of the disease or disorder.

Non-limiting examples of “sample” include any material from which nucleic acids and/or proteins can be obtained. As non-limiting examples, this includes whole blood, peripheral blood, plasma, serum, cerebral spinal fluid, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In some embodiments, the sample comprises tissue from the CNS. In some embodiments, the sample comprises white blood cells. Alternatively, a sample can be obtained through primary patient derived cell lines, or archived patient samples in the form of preserved samples, or fresh frozen samples.

In some embodiments, the terms “polypeptide,” “peptide,” and “protein” are used interchangeably herein to refer to polymers of amino acids of any length. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component.

As used herein, the term “percent (%) identity”, or “percent sequence identity,” with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. As used herein, the term “percent (%) identity”, or “percent sequence identity,” with respect to a reference nucleic acid sequence is the percentage of nucleotides in a candidate sequence that are identical with the nucleotides in the reference nucleic acid sequence, after aligning the sequences and introducing gaps, if necessary, to achieve the maximum percent sequence identity. Alignment for purposes of determining percent sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

A “vector” as used herein refers to a macromolecule or association of macromolecules that comprises or associates with a polynucleotide and which can be used to mediate delivery of the polynucleotide to a cell. Examples of vectors include plasmids, viral vectors, liposomes, and other gene delivery vehicles.

The term “AAV” is an abbreviation for adeno-associated virus and refers to the virus itself or derivatives thereof. The term covers all subtypes and both naturally occurring and recombinant forms, except where required otherwise. The term “AAV” includes AAV types 1 to 12, AAV-DJ, AAV3B, AAV5, AAV8, AAV9, scAAV3,B scAAV5, scAAV8, scAAV9, avian AAV, bovine AAV, canine AAV, equine AAV, primate AAV, non-primate AAV, and ovine AAV.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., a level of mRNA expression or a level of protein expression), or a rate of production (e.g., a rate of mRNA expression between two points in time or a rate of protein expression between two points in time).

The terms, “decreased” or “decrease”, are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

In some embodiments, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

As used herein, “operably linked” or “operable linkage” of a nucleic acid to a regulatory element (e.g., effector sequences of nucleotides, such as promoters or enhancers) refers to the relationship between such nucleic acid, such as DNA, and such sequences of nucleotides. Thus, operatively linked refers to the functional relationship of nucleic acid, such as DNA, with regulatory and effector sequences of nucleotides, such as promoters or enhancers. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA.

The term “biomarker” comprises a measurable substance in a subject whose presence, level, or activity, is indicative of a phenomenon (e.g., phenotypic expression or activity; disease, condition, subclinical phenotype of a disease or disorder, infection; or environmental stimuli). In some embodiments, a biomarker comprises a gene, gene expression product (e.g., RNA or protein), or a cell-type (e.g., microglia).

The section headings used herein are for organizational purposes only and are not to be construed as limiting the subject matter described.

### EXAMPLES

The following illustrative examples are representative of embodiments of the stimulation, systems, and methods described herein and are not meant to be limiting in any way.

### Example 1: Specificity of RGS10 in Microglia

RGS10 is known to be expressed in many cell types of different tissues. However, RGS10 expression is not ubiquitous among all cell types. Additionally, in comparing some tissue types, cell types, or cell states in which RGS10 is naturally expressed, the expression level of RGS10 is often significantly different between various tissue types, cell types, or cell states. This indicates that dynamic regulation of RGS10 expression depending on cell type and physiological context is a component of the endogenous function roles that RGS10 facilitates. As such, the specificity for RGS10 expression in microglia was determined. The relative mRNA abundance of RGS10 was assayed in primary microglia from mice and compared to the mRNA expression of other RGS family members.

First, primary microglial cells were isolated from mice (C57BL/6, postnatal day 2-4 pups, 500,000 cells) and aliquoted as samples according to the protocol in Lee J K. Chung J. McAlpine F E. Tansey M G. J Neurosci. 2011;31(33):11879-11888. Next, total RNA was extracted from primary microglial cell samples and reverse transcribed into cDNA. Quantitative RT-PCR (qRT-PCR) using a SYBR green assay was used with primers allowing specific amplification of RGS1, RGS2, RGS4, RGS5, RGS7, RGS8, RGS9, RGS10, RGS11, RGS12, RGS13, RGS14, RGS16, RGS17, RGS18, RGS19, RGS20, and GAPDH cDNAs from the samples. Primers used in the qRT-PCR assay are listed in Table 4. Relative abundances detected of each RGS cDNA in the samples were normalized against the value for GAPDH as a housekeeping expression control. The measured relative abundance of RGS1 was converted to a value of 1.0 and the measured relative abundances of other RGS cDNAs were calculated in relation to the RGS1 expression levels. The results were graphed in FIG. 1.

Conclusions: RGS10 was the most prominently expressed RGS family member in microglia and RGS10 expression level was significantly higher than the expression levels of all other RGS family members. RGS1 and RGS19 displayed less robust, but still detectable, expression levels in microglia compared to RGS10. RGS2, RGS4, RGS5, RGS7, RGS8, RGS9, RGS11, RGS12, RGS13, RGS14, RGS16, RGS17, RGS18, and RGS20 displayed fractional or non-detectable expression levels in microglia compared to RGS10. This indicated that RGS10 would be the most abundant RGS protein in brain microglia.

TABLE 4
Primers used in RGS family member qRT-PCR

| | SEQ | |
|---|---|---|
| Sequence | ID | |
| Name | NO: | Sequence |
| mRGS1-QF | 20 | CGGCCAAGTCCAAAGACATAC |
| mRGS1-QR | 21 | TGTCTGGTTGGCAAGGAGTTT |
| mRGS2-QF | 22 | ATCAAGCCTTCTCCTGAGGAA |
| mRGS2-QR | 23 | GCCAGCAGTTCATCAATGC |
| mRGS4-QF | 24 | GGGCTGAATCGTTGGAAAAC |
| mRGS4-QR | 25 | ATTCCGACTTCAGGAAAGCTTT |
| mRGS5-QF | 26 | GCGGAGAAGGCAAAGCAA |
| mRGS5-QR | 27 | GTGGTCAATGTTCACCTCTTTAGG |
| mRGS7-QF2 | 28 | GGGAGCCAGAAAACACAGACT |
| mRGS7-QR2 | 29 | TTGCCTTGTTTTGCATTGTTC |
| mRGS8-QF | 30 | GAGCAAGCATCTCTGACCATCT |
| mRGS8-QR | 31 | ACTGTCGTTGACTGGTCCTCAT |
| mRGS9-QF | 32 | CACCCAGCGCCTTTGG |
| mRGS9-QR | 33 | GACAATAAGGTTGCCCAGGTT |
| mRGS10-QF | 34 | TTGGCTAGCGTGTGAAGATTTC |
| mRGS10-QR | 35 | TGGCCTTTTCCTGCATCTG |
| mRGS11-QF3 | 36 | TGCTGAGCTAGACTATGCCATCT |
| mRGS11-QR3 | 37 | CTAAAGCCCCTTGTTTTTGGA |
| mRGS12-QF | 38 | GCTGGATCTCGTTCCGATTAA |
| mRGS12-QR | 39 | CCGCAACACTTCCGTGACT |
| mRGS13-QF | 40 | ATTATACAGACAGTGGCGTGTTTTT |
| mRGS13-QR | 41 | AAATTGCACGACACACCAAGTAC |
| mRGS14-QF | 42 | GCCCCGGCCAGATATGT |
| mRGS14-QR | 43 | GCGCATAGCTGTCGAACTTC |
| mRGS16-QF | 44 | CCTGGTACTTGCTACTCGCTTTT |
| mRGS16-QR | 45 | AGCACGTCGTGGAGAGGAT |
| mRGS17-QF | 46 | GGAGAGCATCCAGGTCCTAGA |
| mRGS17-QR | 47 | TGAGACCAGGACAAGACTTCATC |
| mRGS18-QF | 48 | CTTCTCCTACAGAGCCCTGACTT |
| mRGS18-QR | 49 | GGCCAAGAGGGCAGATCTAC |
| mRGS19-QF | 50 | GCAACCCCCTCCACTCA |
| mRGS19-QR | 51 | TGTGCTGTTTCTCAGCCTCAT |
| mRGS20-QF | 52 | GCCTGCTGCTTCTGTTGGT |
| mRGS20-QR | 53 | GGTCTTCTTGGTTTCTGCCAGTGA |
| mGAPDH QF | 54 | CAAGGTCATCCATGACAACTTTG |
| mGAPDH QR | 55 | GGCCATCCACAGTCTTCTGG |

### Example 2: Microglia Lacking RGS10 Overproduce Proinflammatory Cytokines

BV2 cells are a type of microglial cell line that was originally derived from a C57/B16 mouse. BV2 cells were immortalized by v-raf/v-myc J2 retrovirus. BV2 cells have been extensively studied and have been demonstrated to retain microglia morphology and functional characteristics indicating their utility in serving as a model system for microglia in many experimental settings. In accordance with v-raf/v-myc immortalization, BV2 cells possess proliferation and metabolic rates that exceeds that of many primary microglial cells.

The role of RGS10 in regulating a response to inflammatory and metabolic stresses was assessed in microglia cells. BV2 cells were grown in culture and RGS10 expression in BV2 cells was diminished by CRISPR/Cas9 RGS10 knockout methods. Control cells were obtained from CRISPR/Cas9 controls. BV2 cells with CRISPR/Cas9 RGS10 knockdown (KD) model of microglia showed greatly reduced RGS10 mRNA levels and, consequently, less RGS10 protein produced.

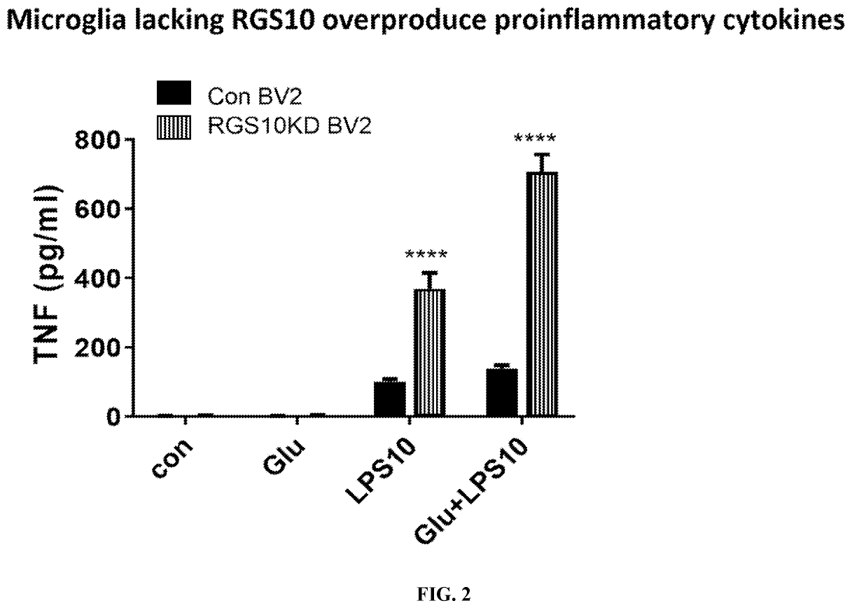

Control and RGS10 KD BV2 cells were allocated into 4 test conditions. The 4 test conditions were 1) Control (no treatment) 2) High glucose treatment (17.5 mM) 3) LPS treatment (10 ng/mL) and 4) High glucose (17.5 mM) plus LPS treatment (10 ng/ml). Cultured cells were subjected to test conditions for 18 hours and then assayed for production of TNF-α using an immunoassay (Meso-Scale Discovery). Cell culture protocols and immunoassays were performed as previously described (Lee J K, McCoy M K, Harms A S, Ruhn K A, Gold S J, Tansey M G. J Neurosci. 2008;28(34):8517-8528). Results were graphed in FIG. 2.

Conclusions: Control and RGS10 KD microglia exhibited low level production of the proinflammatory cytokine TNF-α under Condition 1) entailing no treatment. Condition 2) modeled metabolic stress and Control and RGS10 KD microglia also exhibited low level production of TNF-α. This demonstrated that metabolic stress on its own did not lead to a proinflammatory response in Control and RGS10 KD microglia represented by elevated TNF-α production. Condition 3) modeled an inflammatory stimulus. Control microglia exhibited elevated production of TNF-α. RGS10 KD microglia exhibited a roughly 3-fold increase in the extent of elevated production of TNF-α as compared to control microglia that was a statistically significant difference. This demonstrated a role for RGS10 in microglia to prevent excessive response to an inflammatory stimulus as measured by TNF-α production. Condition 4) modeled metabolic stress in conjunction with an inflammatory stimulus. Control microglia exhibited elevated production of TNF-α as compared to the no treatment condition indicating that microglia do response to combined metabolic stress and inflammatory stimulus with production of an inflammatory cytokine. RGS10 KD microglia exhibited a roughly 5-fold increase in the extent of elevated production of TNF-α as compared to control microglia that was a statistically significant difference. This demonstrated a role for RGS10 in microglia to prevent a synergistic excessive response to combined metabolic stress and inflammatory stimulus as measured by TNF-α production.

### Example 3: RGS10 KD microglia Are Resistant to Antioxidant-Induced Reduction of an Inflammatory Response

Microglia were assayed for their ability to respond to antioxidant treatment by reducing the extent of a proinflammatory characteristic. In this assay, TNF-α production level served as a marker for an inherent proinflammatory characteristic in microglia cells.

Control and RGS10 KD BV2 cells were allocated into 2 test conditions. The 2 test conditions were 1) Control (no treatment) 2) N-acetylcysteine (NAC) treatment (5 mM). NAC is an antioxidant precursor. NAC is a precursor of L-cysteine that results in biosynthesis of glutathione. Glutathione is a powerful, naturally occurring antioxidant. NAC also acts directly as a scavenger of free radicals, especially oxygen radicals, illustrating extensive antioxidant properties of NAC treatment. NAC treatment in cells leads to a reduction in reactive oxidative species (ROS).

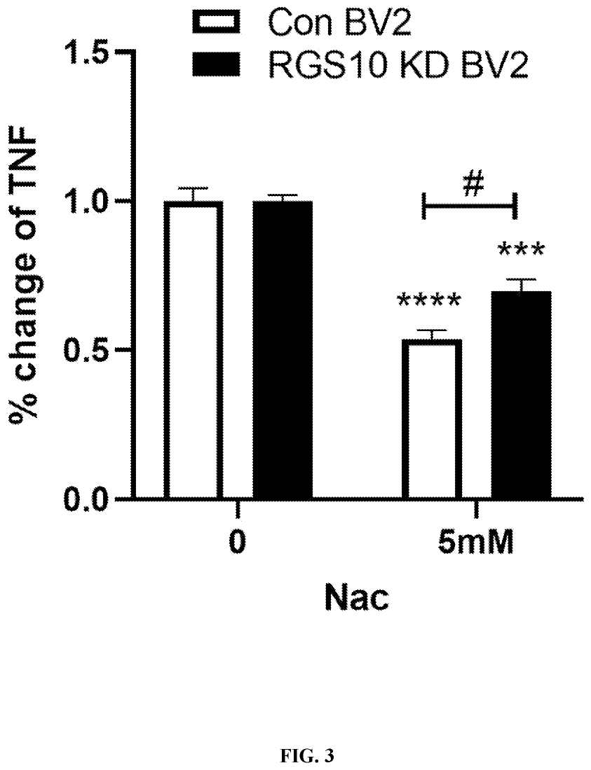

Cultured cells were subjected to test conditions for 18 hours and then assayed for production of TNF-α using an immunoassay (Meso-Scale Discovery). Values for TNF-α production in Control and RGS10 KD BV2 cells under Condition 1) were scaled to value of 1.0. Values for TNF-α production after NAC treatment were represented as a % of change relative to Condition 1) levels and were graphed in FIG. 3.

Conclusions: Control and RGS10 KD microglia exhibited low level production of the proinflammatory cytokine TNF-α under Condition 1) entailing no treatment which represented an inherent proinflammatory characteristic in microglia cells. Under Condition 2), Control microglia responded to NAC treatment with a statistically significant reduction in level of TNF-α production. Under Condition 2), RGS10 KD microglia also responded to NAC treatment with a statistically significant reduction in level of TNF-α production compared to Condition 1). Therefore, RGS10 KD microglia were still capable of responding to antioxidant treatment to reduce a proinflammatory characteristic. Notably, the extent of the response to antioxidant treatment to reduce a proinflammatory characteristic was greatly reduced in RGS10 KD microglia compared to Control microglia. This was a statistically significant difference in response. These results demonstrated that RGS10 KD in microglia leads to increased ROS. This increase in ROS in RGS10 KD microglia reflected increased oxidative stress which could lead to increased risk or severity of a disease or disorder causatively linked to increased oxidative stress. This increase in oxidative stress in RGS10 KD microglia could also inhibit a natural functional role for RGS10 in microglia for maintaining protection of neurons from the damaging effects of insults such as oxidative stress, metabolic stress, and inflammation.

### Example 4: Microglia Lacking RGS10 Display Impaired Clearance of Alpha-Synuclein Aggregates

Microglia were assayed for their ability to clear alpha-synuclein (α-Syn) aggregates. In this assay, measurements were conducted under various test conditions on the extent of intracellular α-Syn. α-Syn aggregates that had been taken up by microglia cells were measured by Western blot.

Control and RGS10 KD BV2 cells cultured according to (Lee J K, Chung J, McAlpine F E, Tansey M G. J Neurosci. 2011;31(33):11879-11888) and were allocated into 2 test conditions. The 2 test conditions were 1) Control (no treatment) and 2) addition of extracellular α-Syn aggregates to the growth media (10 μg/mL). Samples were subjected to either Condition 1) or Condition 2) for 1 hour. Cells were then washed to remove excess α-Syn that was not taken up by microglial cells. After sufficient washes, cells were collected, lysed, and the protein contents were assayed by Western blot.

Western blot procedure. Cells were lysed with 1% NP-40, 10 mM Tris, pH 7.4, 150 mM NaCl, 100 μg/ml PMSF, and protease inhibitor mix (Sigma) for 30 min on ice. Lysates were resuspended in 2× Laemmli sample buffer and loaded on precast 12% SDS-PAGE gels (Bio-Rad), transferred onto PDVF membranes (Millipore), and probed with anti-α-Syn (100 ng/mL) (Abcam) or anti-β-actin (200 ng/mL) antibody (Santa Cruz Biotechnology, Inc.) plus the appropriate HRP-conjugated secondary antibody (1:5000, Jackson ImmunoResearch Laboratory). Immunoreactive bands were visualized with SuperSignal West Femto HRP substrate (Thermo Fisher Scientific) according to the manufacturer's instructions. Membranes were stripped with 0.2 M glycine, 1% SDS and 0.1% Tween 20, pH 2.2 and reprobed as necessary.

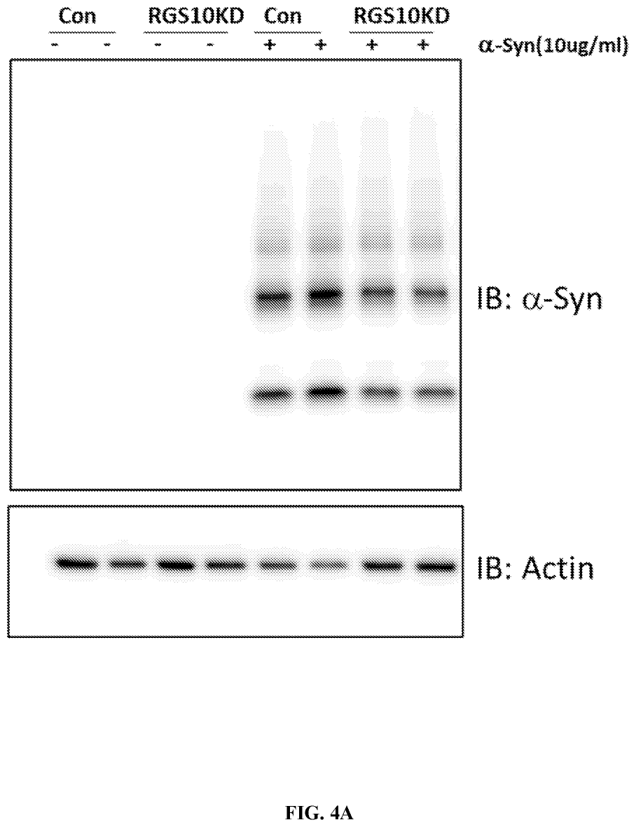

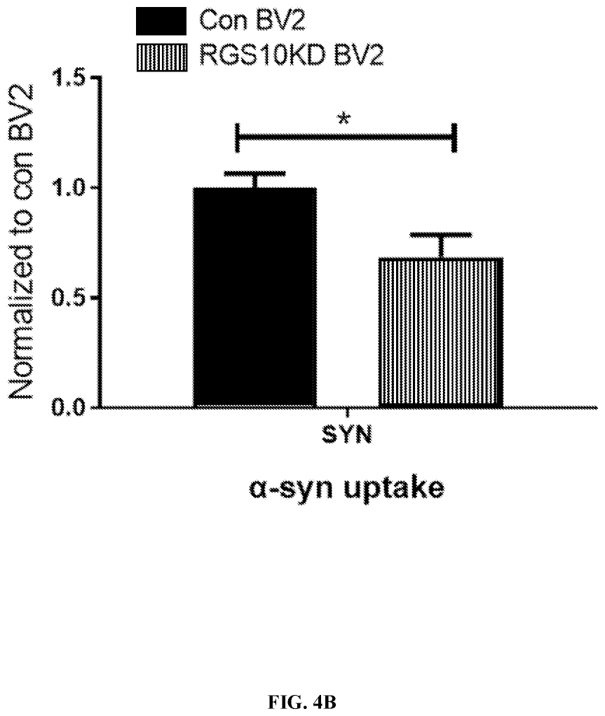

Western blot results are displayed in FIGS. 4A-4B. FIG. 4A is an image of the Western blot showing α-Syn that has been take up by microglia. FIG. 4B shows a graph with quantitation of Control and RGS10 KD microglia ability to mediate α-Syn uptake. Sample amount loaded per lane of the blot were normalized to a measurement of β-actin in each sample. Control values for α-Syn were then normalized to a scale of 1.0 and RGS10 KD α-Syn levels were graphed using the same conversion factor for normalization.

Control and RGS10 KD microglia cultured under Condition 1) showed no detectable intracellular α-Syn indicating that BV2 cells do not endogenously produce detectable intracellular α-Syn under these culture conditions. Control and RGS10 KD microglia cultured under Condition 2) both showed robust uptake of α-Syn. FIG. 4B demonstrates that RGS10 KD microglia had a statistically significant reduction in the amount of α-Syn as compared to Control microglia.

Conclusion: RGS10KD microglia displayed impaired phagocytosis against α-syn aggregates.

### Example 5: Aged RGS10 KO Mice Display Increased Weight Gain and Impaired Metabolic Homeostasis

Mice with a targeted mutation at the RGS10 locus were previously produced and determined to be viable in the homozygous state (Lee J K, McCoy M K, Harms A S, Ruhn K A, Gold S J, Tansey M G, J Neurosci. 2008;28(34):8517-8528). As such, these RGS10 knockout (KO) mice can be studied to determine functions for RGS10 in various biological processes and contexts.

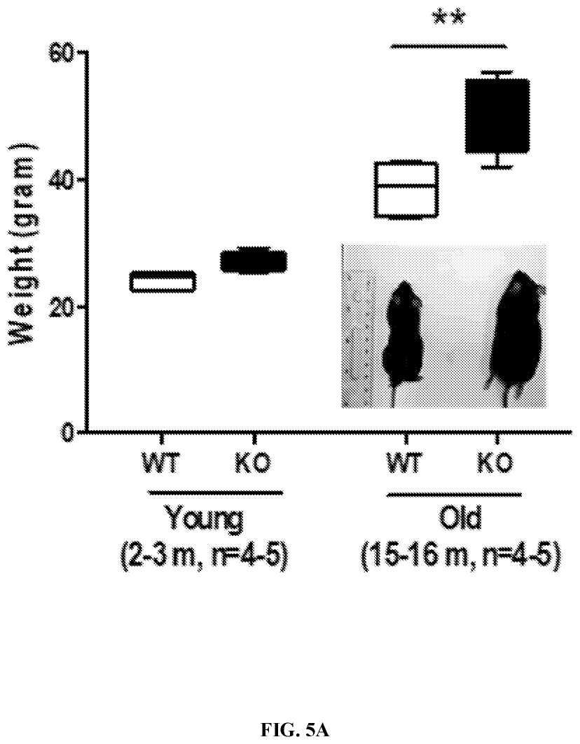

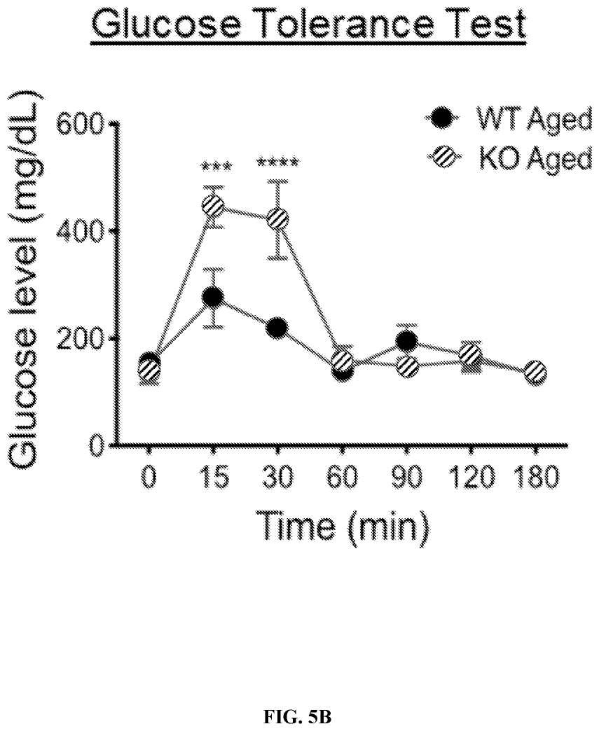

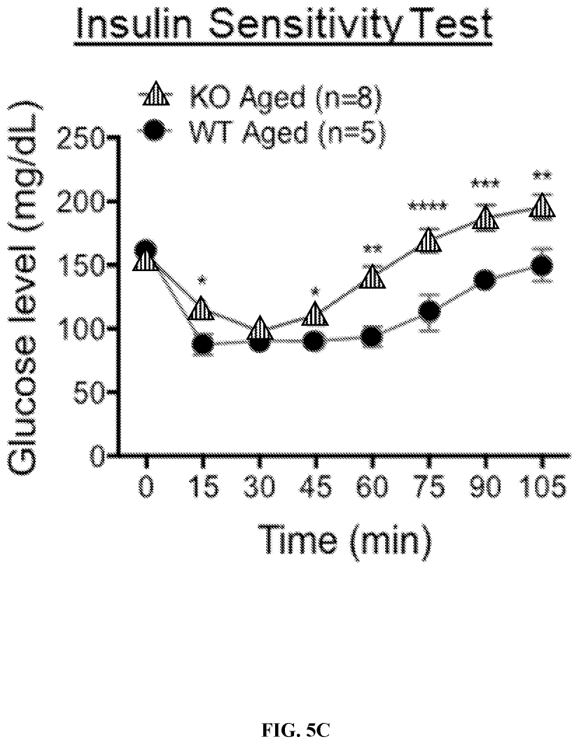

The role of RGS10 in weight control and metabolic homeostasis was assayed. First, weights of wild-type (WT) and RGS10 KO mice were measured and analyzed at different ages. Results are shown in FIG. 5A. Young mice (2-3 months old), showed similar weight profiles with RGS10 KO mice showing a trend toward increased weight compared to WT mice. As the mice aged, this trend continued showing statistical significance in Old mice (15-16 months old). As seen in FIG. 5A, RGS10 old mice were significantly heavier than age-matched WT mice. Next, aged mice (15-16 months old) were subjected to oral glucose tolerance tests (OGTT) and insulin sensitivity tests (IST) following protocols found in (Fang X et al. Depletion of regulator-of-G-protein signaling-10 in mice exaggerates high-fat diet-induced insulin resistance and inflammation, and this effect is mitigated by dietary green tea extract. Nutr Res. 2019 October;70:50-59). OGTT and IST were performed in succession (3 days apart) on the same animals. Mice were fasted for 3 hours prior to the experiments. Following the fasting, glucose (2 g/kg BW, Sigma-Aldrich, St Louis, MO, USA) was administered through oral gavage and blood glucose level was measured using a glucometer (TRUEresult, Nipro Diagnostics, Fort Lauderdale, FL, USA) by serial tail bleeds at various time points (0, 15, 30, 60, 90, and 120 min). Following 3 days of recovery, insulin (0.5 IU/kg, Sigma- Aldrich, St. Louis, MO, USA) was intraperitoneal injected after 3 hours of fasting and blood glucose level was measured at various time points (0, 15, 30, 60, 75, 90, 105, and 120 min. Results of the glucose tolerance tests were graphed in FIG. 5B. RGS10 KO aged mice showed a poor acute response to glucose challenge with significantly elevated blood glucose levels at the 15 and 30 minute time points compared to WT aged mice. Results of the insulin sensitivity tests were graphed in FIG. 5C. RGS10 KO aged mice showed impaired insulin at the majority of time points analyzed with the largest impairment occurring from 60 minutes onward.

Conclusions: RGS10 KO mice demonstrated increased weight with age indicating that metabolic function of RGS10 KO mice is impaired leading to the excessive weight gain. Aged mice also demonstrated an impaired ability to store glucose by removing it from the blood. This effect is seen in Type 2 diabetes. Aged mice also demonstrated impaired insulin resistance. This effect is also seen in Type 2 diabetes. It is possible that increased weight profile for Old mice is the main driver for elevated insulin resistance seen in the Aged mice.

### Example 6: RGS10 Is Directly Involved in Neuronal Activity

The role of RGS10 in regulating learning and memory under conditions of stress was examined. RGS10 KO and WT mice were fed with high fat diet (HFD). To assess learning and memory function, long term potentiation (LTP) was measured as field excitatory postsynaptic potential (fEPSP) in hippocampal neurons on ex vivo brain slices. HFD is known to be stressful to those neurons and decrease fEPSP as a result of impaired LTP. Potential differences between male and female mice were taken into consideration by analyzing the results separately.

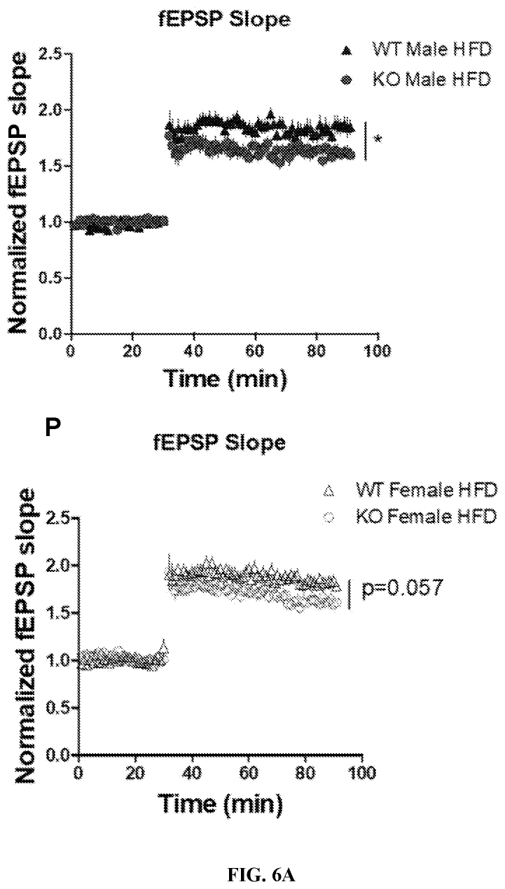

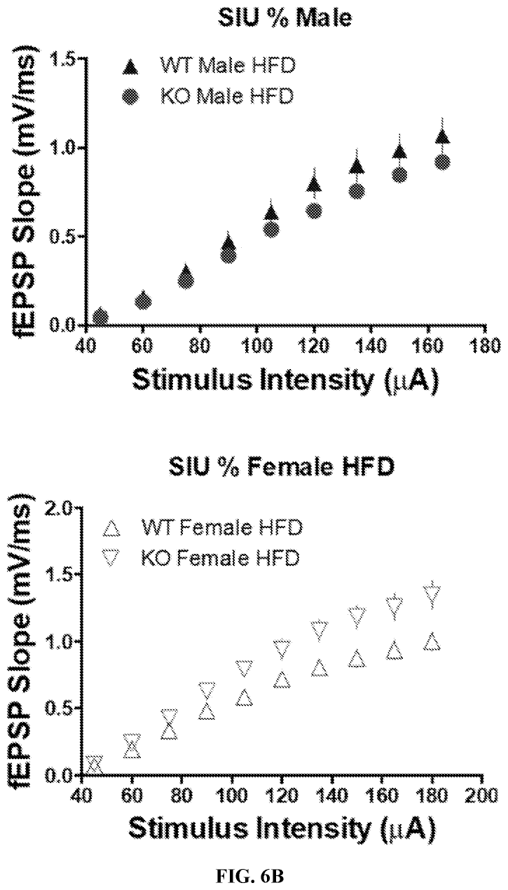

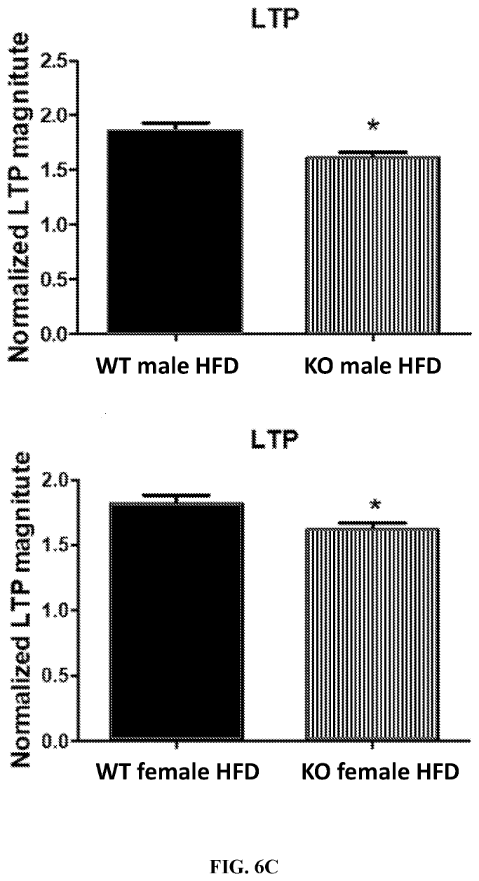

In RGS10 KO mice, fESPS was significantly lower in both male and female (FIG. 6A). The effect of stimulus intensity on fEPSP slope was assayed and graphed in FIG. 6B. At stronger stimulus intensities, the reduction of LTP in RGS10 KO mice was marked greater. FIG. 6C shows graphs of the magnitude of normalized LTP measurements in RGS10 KO and WT mice indicating a statistically significant decrease in LTP in both mice and female RGS10 KO mice subjected to metabolic stress as a result of being fed with HFD.

Conclusions: The results in FIGS. 6A-6C indicated that RGS10 directly regulated synaptic plasticity and ultimately altered learning and memory in response to metabolic and inflammatory stress. These results support the notion that supplementing RGS10 will improve cognitive impairment in patients with neurodegenerative diseases.

### Example 7: Producing AAV-RGS10 Vectors and Validating AAV-Mediated RGS10 Overexpression in a Cell Line

An AAV vector comprising a cell-specific promoter that efficiently drives expression of RGS10 mRNA and protein in microglia is constructed. This AAV vector is transfected in cultured microglial BV2 cells to validate expression of RGS10 protein. A control AAV vector with the same cell-specific promotor is made to express green fluorescent protein (GFP) mRNA instead of RGS10 mRNA.

AAV production: RGS10 cDNA sequence is synthetized and constructed in a plasmid vector with a cell-specific promoter that efficiently drives expression of RGS10 mRNA in microglia thereby increasing expression of RGS10 protein. Cultured cells (e.g., HEK293 cells) are co-transfected with RGS10 plasmid and helper plasmids to package AAV vectors.

BV2 cells are grown in culture and RGS10 mRNA expression is knocked down (KD) by CRISPR/Cas9 methods. Control BV2 cells are transfected with control CRISPR/Cas9 alone. RGS10 KD and control BV2 cells are treated with AAV-RGS10 or AAV-GFP vectors (1×10⁷⁻¹⁰ GC per million cells). Cells are harvested after 3-7 days and subjected to qRT-PCR or western blotting to assay RGS10 mRNA expression or protein levels respectively.

### Example 8: Midbrain Targeting of AAV Expression Vectors and AAV-Mediated RGS10 Overexpression in CNS

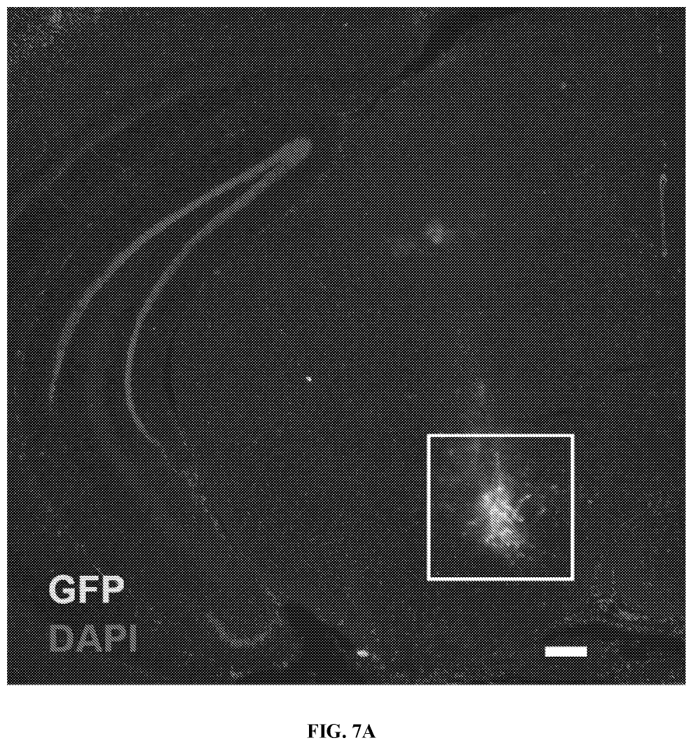

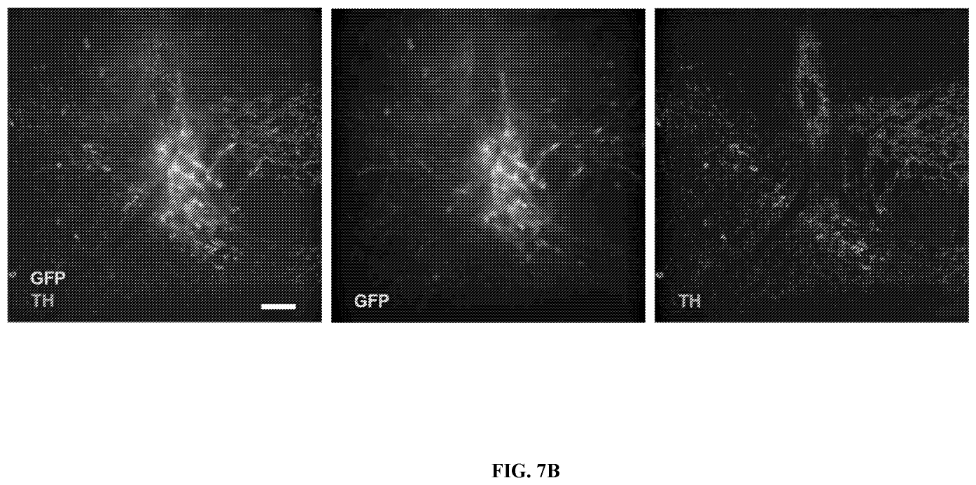

The expression profiles of different AAV serotypes, (e.g., AAV6 vs. AAV9), were investigated in vivo. AAV6-GFP (1 μL of 2×10¹⁰ GC/μL), with GFP expression regulated by a CMV promoter, was injected via a single stereotactic injection into the Substantia nigra pars compacta (SNpc) unilaterally in wildtype (WT) mice (FIGS. 7A-7B). Mice were perfused for histological analysis 3 weeks after injections. GFP expression was assayed by imaging expression pattern and level of GFP fluorescence. GFP expression was found mainly at the injection site of the midbrain (FIG. 7A). Extensive spreading of GFP expression beyond the injection site was not observed following AAV6-GFP injection. Tyrosine hydroxylase (TH) cell labeling was assayed by fluorescent immunohistochemistry by utilizing an anti-TH antibody. Fluorescent immunohistochemistry for TH shows that GFP expressing cells were located within the TH-positive dopaminergic neurons (FIG. 7B). Panels in FIG. 7B were separated by channel of fluorescence signal. Left panel includes GFP and TH channels as an overlay. Center panel shows channel for GFP only. Right panel shows channel for TH only.

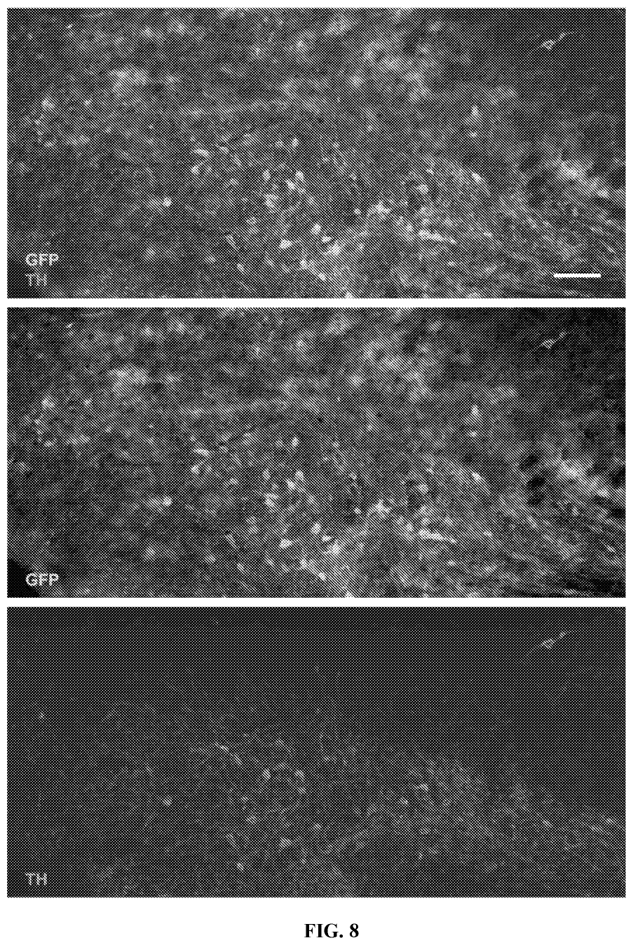

AAV9-GFP, with GFP expression regulated by a CMV promoter, at the same titer as AAV6-GFP was injected into the same brain area of WT mice. After 4 weeks following injection, mice were perfused for histological analysis. GFP expression was assayed by imaging expression pattern and level of GFP fluorescence. Tyrosine hydroxylase (TH) cell labeling was assayed by fluorescent immunohistochemistry by utilizing an anti-TH antibody. Unlike the results observed from AAV6-GFP injected mice, GFP expression in AAV9-GFP injected mice showed extensive spreading of GFP signal across the midbrain including into the dorsal midbrain as is seen by comparing GFP expression to the location of TH-positive dopaminergic neurons in FIG. 8. Panels in FIG. 8 were separated by channel of fluorescence signal. Top panel in FIG. 8 includes GFP and TH channels as an overlay. Center panel in FIG. 8 shows channel for GFP only. Bottom panel in FIG. 8 shows channel for TH only. GFP expression was noted having spread dorsally in the midbrain beyond close proximity to the injection site and beyond labeled TH-positive stained dopaminergic neurons. The extensive spreading of marker gene expression into areas of midbrain not in close proximity to the injection site indicates the effectiveness of AAV9 vectors to disperse expression of a target gene of interest over a larger region of the CNS than was observed using an AAV6 vector. The role of the specific AAV serotype in yielding this unexpected result of widespread expression was observed as the marker gene (GFP) was regulated by the same promoter sequence (CMV) in both the AAV6-GFP vector and the AAV9-GFP vector and the assessment of potential spreading of expression away from the injection site was assayed at similar time points (3 weeks post injection for AAV6-GFP versus 4 weeks post injection for AAV9-GFP). The extensive spreading of marker gene expression in AAV9-GFP resulted in much higher expression levels in AAV9-GFP injected mice compared to AAV6-GFP injected mice following injection with an equivalent volume and titer of vector.

Alternatively in this example, GFP expression is assayed by fluorescent immunohistochemistry for cell labeling and expression level quantitation by utilizing an anti-GFP antibody.

An AAV vector comprising a cell-specific promoter that efficiently drives expression of RGS10 protein in microglia was constructed. This AAV vector was administered to a subject (e.g., a rat or mouse) by a single stereotactic injection into the Substantia nigra pars compacta (SNpc) unilaterally.

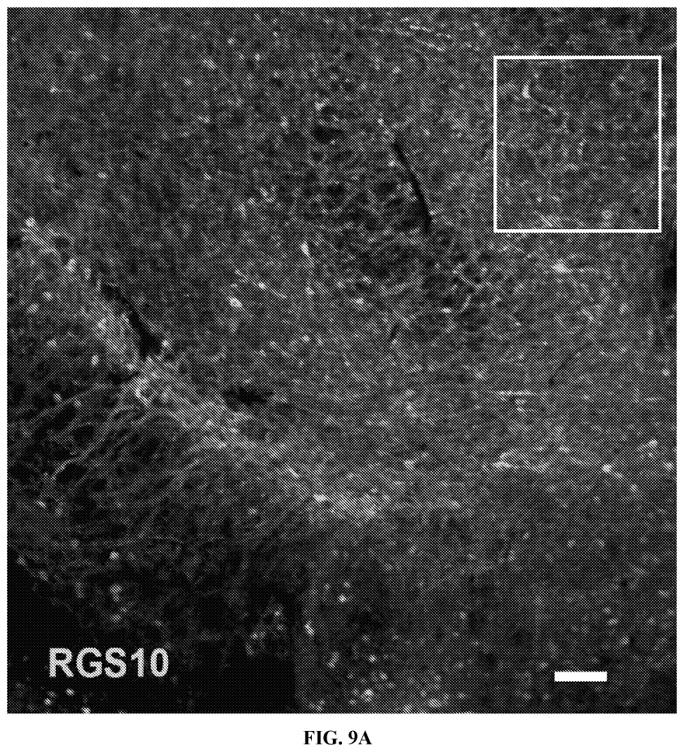

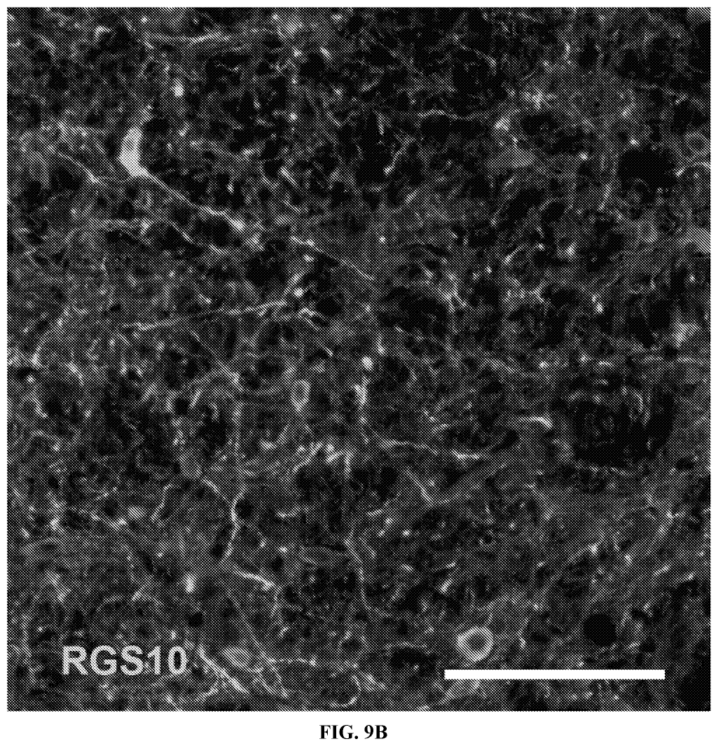

AAV9-RGS10, with RGS10 cDNA expression regulated by a CMV promoter, was produced using the same plasmid vectors and packaging as for AAV9-GFP. The RGS10 cDNA sequence used in this vector comprises the sequence of SEQ ID NO: 1. To ensure AAV-mediated RGS10 expression, RGS10 knock out (KO) mice lacking endogenous RGS10 expression were injected with AAV9-RGS10 (1 μL of 1×10¹⁰ GC/μL). Mice were sacrificed 4 weeks after injections. Immunohistochemistry for RGS10 showed widespread AAV-mediated RGS10 expression in the target brain areas (FIGS. 9A-9B). AAV-mediated RGS10 expression is visualized throughout the midbrain in FIG. 9A. FIG. 9B provides a higher magnification of the white boxed area in FIG. 9A to illustrate subcellular localization of AAV-mediated RGS10 expression in the CNS cells of the midbrain.

Following perfusion, animals are assayed for RGS10 protein expression in microglia cells via fluorescent immunohistochemistry by co-labeling tissue sections with an RGS10 antibody and a microglia-specific marker antibody (e.g., TMEM119, or CD11b and CD45) using immunofluorescent detection and then visualizing the results. RGS10 protein expression level is quantitated by measuring immunofluorescence level in the RGS10 fluorescence channel. Additionally or alternatively, RGS10 protein expression level is quantitated by Western blot.

The following procedures are used for RGS10 expression quantification. Expression of RGS10 is quantified as previously described and whose contents are incorporated by reference with regard to protocols and procedures to be used to determine levels of RGS10 expression (Lee J K et al. Regulator of G-protein signaling-10 negatively regulates NF-κB in microglia and neuroprotects dopaminergic neurons in hemiparkinsonian rats. J Neurosci. 2011 Aug. 17;31(33):11879-88). RGS10 expression is analyzed on images taken with a fluorescence microscope under a 20× objective lens. To achieve this, ImageJ software is used and thresholding analysis is applied to calculate the gray values indicating the intensity of RGS10 expression in each field. This analysis is performed on six distinct brain sections, with 4-6 randomly selected fields per section, from both the injection side and equivalent regions of the contralateral side. Each group of different AAV serotypes or promoters involves 3 RGS10 KO mice. Four groups of different AAV serotypes or promoters are tested. In Group 1 subjects, AAV6-mouse Iba1 promoter-RGS10 (1 μL of 2×10¹⁰ GC/μL), with RGS10 expression regulated by a mouse Iba1 promoter, RGS10 expression distribution and quantitative expression levels are tested. In Group 2 subjects, AAV9-mouse Iba1 promoter-RGS10 (1 μL of 2×10¹⁰ GC/μL), with RGS10 expression regulated by a mouse Iba1 promoter, RGS10 expression distribution and quantitative expression levels are tested. In Group 3 subjects, AAV6-CMV promoter-RGS10 (1 μL of 2×10¹⁰ GC/μL), with RGS10 expression regulated by a CMV promoter, RGS10 expression distribution and quantitative expression levels are tested. In Group 4 subjects, AAV9-CMV promoter-RGS10(1 μL of 2×10¹⁰ GC/μL), with RGS10 expression regulated by a CMV promoter, RGS10expression distribution and quantitative expression levels are tested.

Additionally or alternatively, stereology analysis is performed for RGS10-expressing cells to determine RGS10 expression level quantification. StereoInvestigator analysis software (MicroBrightField Inc.) is utilized to conduct unbiased stereological cell counts of RGS10-labeled cell bodies in the Substantia Nigra pars compacta (SNpc) using the optical fractionator method whose protocols and methods for stereology analysis are herein incorporated by reference (Lee J K et al. Regulator of G-protein signaling-10 negatively regulates NF-κB in microglia and neuroprotects dopaminergic neurons in hemiparkinsonian rats. J Neurosci. 2011 Aug. 17;31(33):11879-88 and West M J et al., Unbiased stereological estimation of the total number of neurons in the subdivisions of the rat hippocampus using the optical fractionator. Anat Rec. 1991 December;231(4):482-97). To ensure impartiality, the treatment of the various brain sections is blinded to the observer. For systematic analysis, serial sections spanning the extent of the SNpc are cut on a cryostat and mounted six per slide, with a cut thickness of 30 μm and a final mounted thickness of 22 μm. To facilitate unbiased sampling, counting frames of size 50×50 μm are randomly placed on a counting grid measuring 190×130 μm. An 18 μm optical dissector with 2 μm upper and lower guard zones is used for sampling. Every other slide is stained for RGS10 and DAPI to facilitate the identification of relevant cells. The boundary of the SNpc is outlined at 4× magnification. Cell counting is performed using a 40× oil-immersion objective lens.

Conclusions: These data collectively demonstrated that AAV-RGS10 dispersed across different distances from the injection site depending on serotypes, transduced different cell types, and expressed RGS10 in TH neurons and non-TH neural cells. AAV9-based vectors yielded delivery of a higher overall level of expression of a target cDNA of interest (e.g., RGS10) over a more extensive area of CNS following focal injection into the substantia nigra pars compacta (SNpc). The demonstrated targeted AAV-mediated RGS10 expression may be sufficient for therapeutic applications described herein.

Injection protocol used in FIGS. 7A-9B: The stereotaxic surgery protocol is followed according to (Lee J K, Chung J, McAlpine F E, Tansey M G. J Neurosci. 2011;31(33):11879-11888).

For this example, 1-2 μL of AAV-RGS10 vector is injected at a final viral titer of between 0.5-2.5×10⁸⁻¹⁰ IU/mL. In mice tested for FIGS. 9A-9B, 1 μL of 1×10¹⁰ GC/μL of AAV9-RGS10 vector was injected.

### Example 9: Effects of RGS10 Overexpression in PFF Model

Given that idiopathic Parkinson's disease (PD) is not generally associated with an increase in total α-syn protein levels, synucleinopathy that arises in the context of normal endogenous α-syn levels could be a relevant model in which to test the effects of increasing RGS10 expression in improving the symptoms associated with PD or providing a measurable extent of neuronal protection from neurodegeneration. In idiopathic PD, decreases in soluble monomeric α-syn with concurrent increases in soluble phosphorylated α-syn (pSyn) along with increases in membrane-bound α-syn have been observed. The α-syn preformed fibril (PFF) model represents an approach in which synucleinopathy is induced in an environment of normal α-syn protein levels. This model is adapted for use both in vitro and in vivo (mouse and rats).

Following a protocol provided in (Duffy M F et al. J Neuroinflammation. 2018 May 1;15(1):129. doi: 10.1186/s12974-018-1171-z. Erratum in: J Neuroinflammation. 2018 May 29;15(1):169) α-syn preformed fibrils are internalized by neurons, recruit endogenous α-syn and accumulate as inclusions of insoluble pSyn. The pSyn inclusions eventually lead to neuronal dysfunction and degeneration in rat neurons.

An AAV vector comprising a cell-specific promoter that efficiently drives expression of RGS10 mRNA and protein in microglia is constructed. This AAV-RGS10 vector is administered to a subject (e.g., a rat PFF model of a synucleinopathy) by a single stereotactic injection into the Substantia nigra pars compacta (SNpc) unilaterally.

Following a period of 3 weeks, animals are perfused and assayed for RGS10 mRNA or protein expression levels in microglia. Dopaminergic fibers were identified and quantitated after immunohistochemical detection of tyrosine hydroxylase (TH) in sections from rat SNpc.

This example demonstrates that an AAV vector can efficiently deliver and induce expression of an untagged version of RGS10 mRNA and protein at a targeted location within the CNS and that this elevated RGS10 expression can mitigate the loss of dopaminergic neurons in a rat model of Parkinson's disease that models some observed synucleinopathies as having an altered ratio of soluble to insoluble α-syn but not an overall increase in α-syn levels that accompany the neurodegeneration.

### Example 10: AAV-Mediated Delivery of RGS10 Using a Microglia-Specific Promoter

To develop microglial expression of RGS10, a microglia-specific promoter is used in an AAV plasmid construct. Ionized calcium-binding adapter molecule 1 (IBA1), also known as Allograft inflammatory factor 1 (AIF-1), is a protein encoded by the AIF1 gene within the major histocompatibility complex class III region on chromosome 6 in humans. IBA1 is specifically expressed in macrophages/microglia and is upregulated during the activation of these cells.

Mouse Iba1 promoter comprising a 1,678-bp fragment from the 5′-flanking region and exon 1 of the mouse Iba1 (Aif1) gene is used to construct an AAV vector operatively-linked to an RGS10-encoding sequence. This mouse promoter region sequence drives transcription of an operatively-linked target sequence of interest (e.g., a sequence encoding an RGS10 protein, or functional fragment thereof) in microglia cells. Mouse Iba1 promoter sequence for use in this construct is described in Table 3. This AAV vector can drive transcription of RGS10 in rodent and human microglia cells. The mouse Iba1 promoter sequence for use in this construct is prepared in an AAV6 serotype vector (AAV6-mouse Iba1 promoter-RGS10). Separately, the mouse Iba1 promoter sequence for use in this construct is prepared in an AAV9 serotype vector (AAV9-mouse Iba1 promoter-RGS10). The sequence encoding an RGS10 protein, or functional fragment thereof for use in both constructs is selected from SEQ ID NO: 1-5.

Alternatively, human Aif-1 promoter is used to construct an AAV vector directing microglial expression of RGS10. The Aif-1 promoter has been identified and characterized for potential binding sites and a 1,760-bp fragment has been characterized to analyze transcription variations. Human IBA1 promoter sequence for use in this construct is described in Table 3. The human IBA1 promoter sequence for use in this construct is prepared in an AAV6 serotype vector (AAV6-human IBA1 promoter-RGS10). Separately, the human IBA1 promoter sequence for use in this construct is prepared in an AAV9 serotype vector (AAV9-human IBA1 promoter-RGS10). The sequence encoding an RGS10 protein, or functional fragment thereof for use in both constructs is selected from SEQ ID NO: 1-5.

Alternatively, any one promoter sequence described in Table 3, or a sequence at least 90% identical to a contiguous portion of a sequence listed in Table 3, may be operatively-linked to an RGS10-encoding sequence to construct an AAV vector capable of transcribing RGS10 in microglia following administering of the AAV-RGS10 construct to a subject.

A comparison in targeted RGS10 expression in specific cell types following administration of a microglia-specific promoter driven AAV vector is undertaken. The expression profiles of different AAV serotypes, (e.g., AAV6 vs. AAV9), driven by a microglia-specific promoter are investigated in vivo. In Group 1 subjects, AAV6-mouse Iba1 promoter-RGS10 (1 μL of 2×10¹⁰ GC/μL), with RGS10 expression regulated by a mouse Iba1 promoter, is injected via a single stereotactic injection into the Substantia nigra pars compacta (SNpc) unilaterally in RGS10 KO mice. In Group 2 subjects, AAV9-mouse Iba1 promoter-RGS10 (1 μL of 2×10¹⁰ GC/μL), with RGS10 expression regulated by a mouse Iba1 promoter, is injected via a single stereotactic injection into the Substantia nigra pars compacta (SNpc) unilaterally in RGS10 KO mice. Mice are sacrificed 4 weeks after injections. Immunohistochemistry for RGS10 is performed to determine extent of spreading and level of expression in the target brain areas. AAV-mediated RGS10 expression is visualized throughout the midbrain. A comparison is made between Group 1 and Group2 subjects to determine the results of spreading and expression levels of RGS10 by group. Group 1 and Group 2 subjects are further assayed for AAV-mediated expression of RGS10by cell type. Coronal sections of midbrain are assayed by double-label fluorescent immunohistochemistry thereby labeling individual cells using RGS10 antibody together with antibodies selected from the following list of specific cell type markers:
NeuN—mature neurons (e.g., post-mitotic neurons), excluding Golgi cells, Purkinje cells, Olfactory bulb mitral cells, Retinal photoreceptor cells, Inferior olivary and dentate nucleus neurons, and Sympathetic ganglion cells
MAP2—mature neurons (e.g., post-mitotic neurons)
GFAP—astrocytes
Iba1—microglia
Trem2—microglia.

To assess cell-specificity of viral expression of RGS10, tissues are fixed and processed for immunohistochemistry (IHC). Sections are labeled with RGS10 antibodies along with one of microglia (Iba1), astrocyte (GFAP), and neuron (MAP-2) markers. Fluorescence imaging analysis is analyzed for the intensity of RGS10 expression above a threshold level in each neural cell type, using a double channel threshold method using RGB threshold function at ImageJ software.

A further comparison of targeted RGS10 expression in specific cell types following administration of a microglia-specific promoter driven AAV vector compared to targeted RGS10 expression in specific cell types following administration of a CMV promoter driven AAV vector is undertaken. Group 1 and Group 2 subjects are tested and assayed as above in this example.

In Group 3 subjects, AAV6-CMV promoter-RGS10 (1 μL of 2×10¹⁰ GC/μL), with RGS10 expression regulated by a CMV promoter, is injected via a single stereotactic injection into the Substantia nigra pars compacta (SNpc) unilaterally in RGS10 KO mice. In Group 4 subjects, AAV9-CMV promoter-RGS10 (1 μL of 2×10¹⁰ GC/μL), with RGS10 expression regulated by a CMV promoter, is injected via a single stereotactic injection into the Substantia nigra pars compacta (SNpc) unilaterally in RGS10 KO mice. Mice from Groups 1-4 are sacrificed 4 weeks after injections. Immunohistochemistry for RGS10 is performed to determine extent of spreading and level of expression in the target brain areas. AAV-mediated RGS10 expression is visualized throughout the midbrain. A comparison is made between Group 1, Group 2, Group 3, and Group 4 subjects to determine the results of spreading and expression levels of RGS10 by group. Group 1, Group 2, Group 3, and Group 4 subjects are further assayed for AAV-mediated expression of RGS10 by cell type. Coronal sections of midbrain are assayed by double-label immunohistochemistry thereby labeling individual cells using RGS10 antibody together with antibodies selected from the following list of specific cell type markers:
NeuN—mature neurons (e.g., post-mitotic neurons), excluding Golgi cells, Purkinje cells, Olfactory bulb mitral cells, Retinal photoreceptor cells, Inferior olivary and dentate nucleus neurons, and Sympathetic ganglion cells
MAP2—mature neurons (e.g., post-mitotic neurons)
GFAP—astrocytes
Iba1—microglia
Trem2—microglia.

Results are compared between Groups 1-4 by identified cell type either expressing or not expressing AAV-mediated RGS10 to determine efficiency of targeting AAV-mediated RGS10 expression in mature neurons, astrocytes, and microglia or extent of exclusion of AAV-mediated RGS10 expression in mature neurons, astrocytes, and microglia.

### Example 11: Determination of Doses of AAV-RGS10

To determine the optimal titer for clinical applications, AAV-RGS10 is delivered via intravenous or intrathecal injections. RGS10 KO mice are used to quantify AAV-induced RGS10 levels. The titer level is determined based on the comparison of in vivo transduction efficiency from intranigral injection (1 μL of 2×10¹⁰ GC/μL) of different AAV serotypes (e.g., AAV6 vs. AAV9) and promoters (e.g., CMV vs mouse Iba1). Intrathecal titers are 5×, 50× and intravenous titers are 10×, 100× from intranigral injections. After 2 weeks, mice are sacrificed and microglia are isolated for RT-PCR and western blot analysis to quantify RGS10 expression levels. A titer that restores the baseline level of RGS10 expression in WT microglia is selected for each route.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A method for increasing a Regulator of G-protein signaling 10 (RGS10) protein level in a brain of a subject in need thereof, the method comprising administering an adeno-associated virus (AAV) vector comprising a nucleic acid sequence encoding a RGS10 protein or a functional fragment thereof to the subject, wherein the subject has been diagnosed with or is at risk for developing a neurodegenerative disease.

2. The method of claim 1, wherein the subject has been diagnosed with a neurodegenerative disease.

3. The method of claim 2, wherein the neurodegenerative disease is characterized by an amyloid aggregate pathology.

4. The method of claim 3, wherein the amyloid aggregate pathology is α-synuclein pathology, β amyloid peptide (Aβ) pathology, or a combination thereof.

5. The method of claim 2, wherein the neurodegenerative disease is Parkinson's Disease, Dementia with Lewy Body, Essential Tremor, Multiple System Atrophy, Pure Autonomic Failure, Alzheimer's Disease, or Alzheimer's Disease with Amygdalar Restricted Lewy Bodies (AD/ALB).

6. A method for treating a disease or disorder, the method comprising administering an adeno-associated virus (AAV) vector comprising a nucleic acid sequence encoding a Regulator of G-protein signaling 10 (RGS10) protein or a functional fragment thereof to a subject in need thereof.

7. The method of claim 6, wherein the administering increases expression of RGS10 protein or a functional fragment thereof in a plurality of neurons and a plurality of microglia.

8. The method of claim 6 or 7, wherein the increased expression of RGS10 protein or a functional fragment thereof decreases an amount of secretion of pro-inflammatory cytokines.

9. The method of any one of claims 6-8, wherein the increased expression of RGS10 protein or a functional fragment thereof increases an amount of secretion of a neuroprotective substance.

10. The method of claim 6, wherein the disease or disorder comprises a synucleinopathy.

11. The method of claim 10, wherein the synucleinopathy comprises Parkinson's Disease, Dementia with Lewy Body, Essential Tremor, Multiple System Atrophy, Pure Autonomic Failure, Alzheimer's Disease with Amygdalar Restricted Lewy Bodies (AD/ALB), or any combination thereof.

12. The method of any one of claims 6-11, wherein the administering decreases a level of α-syn aggregates in a brain by at least 5%.

13. The method of claim 12, wherein the level of α-syn aggregates comprises intracellular α-syn aggregates, extracellular α-syn aggregates, or a combination thereof.

14. The method of claim 13, wherein the level of α-syn aggregates is measured quantitatively using an antibody that detects phosphorylated α-syn.

15. The method of claim 14, wherein the disease or disorder comprises a metabolic condition.

16. The method of any one of claims 6-15, wherein the subject has Type 2 diabetes, diabetic neuropathy, or obesity, or any combination thereof.

17. A method for enhancing neuron protection, the method comprising administering an adeno-associated virus (AAV) vector comprising a nucleic acid sequence encoding a Regulator of G-protein signaling 10 (RGS10) protein or a functional fragment thereof to a subject in need thereof.

18. The method of claim 17, further comprising determining a neuron activity in the subject before the administering, after the administering, or before and after the administering.

19. The method of claim 18, wherein the neuron activity is determined by measuring long term potentiation (LTP).

20. The method of claim 19, wherein the neuron activity is determined by measuring a field excitatory postsynaptic potential (fEPSP) in hippocampal neurons.

21. The method of claim 18, wherein the neuron activity is determined through magnetic resonance imaging (MRI).

22. The method of any one of claims 17-21, wherein the subject has a disease or disorder.

23. The method of claim 22, wherein the disease or disorder comprises a synucleinopathy.

24. The method of claim 23, wherein the disease or disorder further comprises Type 2 diabetes, diabetic neuropathy, or obesity, or any combination thereof.

25. The method of claim 23-24, wherein the synucleinopathy comprises Parkinson's Disease, Dementia with Lewy Body, Essential Tremor, Multiple System Atrophy, Pure Autonomic Failure, or Alzheimer's Disease with Amygdalar Restricted Lewy Bodies (AD/ALB).

26. The method of any one of claims 17-25, wherein a quantitative measurement of neuron activity comprises two or more measurements in the subject at different points in time.

27. The method of any one of claims 1-26, wherein a plurality of neurons are protected from a pathological rate of neurodegeneration in the subject.

28. The method of any one of claims 1-27, wherein a plurality of dopaminergic neurons of the midbrain are protected from a pathological rate of neurodegeneration in the subject.

29. The method of any one of claims 1-28, wherein the AAV vector is administered via peripheral injection.

30. The method of any one of claims 1-28, wherein the AAV vector is administered directly to the central nervous system (CNS) of the subject.

31. The method of any one of claims 1-30, wherein the AAV vector is administered directly to the CNS of the subject via intravenous delivery, intravascular delivery, intrathecal delivery, intracisternal delivery, intraspinal delivery, subpial delivery, or intracerebroventricular delivery.

32. The method of any one of claims 1-31, wherein the AAV vector is administered via stereotaxic injection into the brain parenchyma or the spinal cord parenchyma.

33. The method of any one of claims 1-32, comprising delivering the nucleic acid sequence to a plurality of neurons, a plurality of microglia, a plurality of astrocytes or a combination thereof.

34. The method of claim 33, wherein the delivering comprises delivering the nucleic acid sequence to a plurality of microglia.

35. The method of any one of claims 1-34, wherein the administering increases a level of RGS10 protein in a plurality of neurons, a plurality of microglia, a plurality of astrocytes, or a combination thereof by at least 5%.

36. The method of any one of claims 1-35, wherein the administering increases a level of RGS10 protein in a plurality of neurons, in a plurality of microglia, or a combination thereof to an extent that compensates for an age-related decrease in brain RGS10 protein levels.

37. The method of claim 35 or 36, wherein the administering reduces the secretion of proinflammatory cytokines in the plurality of microglia.

38. The method of claim 37, wherein the proinflammatory cytokine is tumor necrosis factor alpha (TNFα).

39. The method of any one of claims 36-38, wherein the administering reduces an amount of intracellular reactive oxygen species (ROS) in the plurality of microglia.

40. The method of any one of claims 1-39, wherein the subject demonstrates an improvement in a result from a glucose tolerance test.

41. The method of any one of claims 1-40, wherein the administering increases long term potentiation (LTP) in a plurality of neurons.

42. The method of claim 41, wherein the increase in LTP is measured in a hippocampus.

43. The method of claim 41 or 42, wherein the increase in LTP is not abrogated by metabolic stress, by inflammatory stress, or by a combination thereof.

44. The method of any one of claims 1-43, wherein the administering results in an improvement of α-syn aggregate pathology in a preformed fibrils (PFFs) model.

45. The method of claim any one of claims 1-44, wherein the AAV vector is selected from AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, and AAV2/AAV12.

46. The method of claim 45, wherein the AAV vector is AAV-9.

47. The method of claim 45, wherein the AAV vector is a single stranded AAV (ssAAV).

48. The method of claim 47, wherein the ssAAV vector comprises a single chain vector of serotype AAV9.

49. The method of any one of claims 45-47, wherein the AAV vector further comprises an AAV capsid.

50. The method of claim 49, wherein the AAV capsid comprises an AAV serotype AAV-9.

51. The method of claim 49, wherein the AAV capsid comprises a pseudotyped AAV capsid.

52. The method of any one of claims 1-51, wherein the AAV vector comprises a cell-specific promoter sequence.

53. The method of claim 52, wherein the cell-specific promoter sequence is a pan promoter.

54. The method of claim 52, wherein the cell-specific promoter sequence is a pan neuronal or neuron subtype-specific promoter.

55. The method of claim 54, wherein the neuron subtype-specific promoter is a dopaminergic neuron-specific promoter.

56. The method of claim 52, wherein the cell-specific promoter sequence is a cell-type specific promoter for microglia.

57. The method of claim any one of claims 52-56, wherein the cell-specific promoter sequence is selected from the group consisting of CamKII, CD68, CMV, F4/80, CX3CR1, CSFR1, Ms4a3, Tmem119, CAG, and IBA1.

58. The method of claim 56, wherein the cell-type specific promoter for microglia comprises an IBA1 promoter.

59. The method of claim 58, wherein the IBA1 promoter is a human IBA1 promoter.

60. The method of claim 58, wherein the IBA1 promoter is a rodent Iba1 promoter.

61. The method of any one of claims 58-60, wherein the IBA1 promoter drives sufficient expression of RGS10 in cells of the CNS.

62. The method of any one of claims 58-61, wherein the IBA1 promoter drives sufficient expression of RGS10 in a plurality of cells of the midbrain.

63. The method of any one of claims 58-62, wherein the IBA1 promoter drives sufficient expression of RGS10 in a plurality of microglia cells of the midbrain.

64. The method of any one of claims 1-63, wherein sufficient expression of RGS10 is maintained for at least 1, 2, 3, 4, 5, 6, 7, 8, 9, 10, 11, or 12 weeks following the administering.

65. The method of any one of claims 1-64, wherein sufficient expression of RGS10 is maintained for at least 4 weeks following the administering.

66. The method of any one of claims 1-65, wherein the nucleic acid sequence comprises a selected from SEQ ID NO: 1-5.

67. The method of any one of claims 1-66, wherein the nucleic acid sequence comprises a sequence that is at least 80%, 85%, 90%, 95% or 99% identical to SEQ ID NO: 1.

68. The method of any one of claims 1-67, wherein the nucleic acid sequence comprises a sequence that is at least 80%, 85%, 90%, 95% or 99% identical to SEQ ID NO: 2 or SEQ ID NO: 4.

69. The method of any one of claims 1-68, wherein the nucleic acid sequence comprises a sequence that is at least 80%, 85%, 90%, 95% or 99% identical to SEQ ID NO: 3 or SEQ ID NO: 5.

70. The method of any one of claims 1-69, wherein the nucleic acid sequence comprises a sequence encoding an amino acid sequence selected from SEQ ID NO: 6-8, or a functional fragment thereof.

71. The method of any one of claims 1-70, wherein the nucleic acid sequence comprises a sequence having at least 80%, 81%, 82%, 83%, 84%, 85%, 86%, 87%, 88%, 89%, 90%, 91%, 92, 93%, 94%, 95%, 96%, 97%, 98%, or 99% identity to a sequence encoding an amino acid sequence selected from SEQ ID NO: 6-8, or a functional fragment thereof.

72. The method of any one of claims 1-71, wherein an RGS10 mRNA expression level is elevated in a plurality of neurons, in a plurality of microglia, or in a combination thereof by at least 5% after the administering of the viral vector.

73. The method of any one of claims 1-72, wherein a level of RGS10 protein in the microglia of the subject is increased by at least 5%, 10%, 20%, 30%, 50%, or 100% after the administering of the viral vector.

74. The method of any one of claims 1-73, wherein the administering comprises confining to or concentrating within one or more targeted sites in the CNS of the subject an RGS10 mRNA sequence by administering an AAV6-RGS10 vector to the one or more targeted sites in the CNS of the subject.

75. The method of any one of claims 1-73, wherein the administering comprises spreading an RGS10 mRNA sequence to one or more CNS regions adjacent to one or more targeted sites in the CNS of the subject by administering an AAV9-RGS10 vector to the one or more targeted sites in the CNS of the subject.

76. An adeno-associated virus (AAV) vector comprising a nucleic acid sequence encoding a Regulator of G-protein signaling 10 (RGS10) protein or a functional fragment thereof.

77. The AAV vector of claim 76, wherein the AAV vector is selected from AAV9, AAV1, AAV2, AAV3, AAV4, AAV5, AAV6, AAV7, AAV8, AAVrh10, AAV11, AAV12, AAV2/1, AAV2/2, AAV2/5, AAV2/6, AAV2/7, AAV2/8, AAV2/9, AAV2/rh10, AAV2/AAV11, AAV-PHP.B, AAV-PHP.EB, and AAV2/AAV12.

78. The AAV vector of claim 77, wherein the AAV vector is AAV-9.

79. The AAV vector of claim 76, wherein the AAV vector is a single stranded AAV (ssAAV).

80. The AAV vector of claim 79, wherein the ssAAV vector comprises a single chain vector of serotype AAV9.

81. The AAV vector of any one of claims 76-80, wherein the AAV vector further comprises an AAV capsid.

82. The AAV vector of claim 81, wherein the AAV capsid comprises an AAV serotype AAV-9.

83. The AAV vector of claim 82, wherein the AAV capsid comprises a pseudotyped AAV capsid.

84. The AAV vector of any one of claims 76-83, wherein the viral vector comprises a cell-specific promoter sequence.

85. The AAV vector of claim 84, wherein the cell-specific promoter sequence is a pan neuronal promoter.

86. The AAV vector of claim 84, wherein the cell-specific promoter sequence is a neuron subtype-specific promoter.

87. The AAV vector of claim 84, wherein the cell-specific promoter sequence is cell-type specific promoter for microglia.

88. The AAV vector of any one of claims 84-87, wherein the cell-specific promoter sequence is selected from the group consisting of CamKII, CD68, CMV, F4/80, CX3CR1, CSFR1, Ms4a3, CAG, and IBA1.

89. The AAV vector of claim 87, wherein the cell-type specific promoter for microglia comprises an IBA1 promoter.

90. The AAV vector of claim 89, wherein the IBA1 promoter is a human IBA1 promoter or a rodent Iba1 promoter.

91. The AAV vector of any one of claims 84-90, wherein the cell-specific promoter sequence comprises a nucleic acid promoter sequence at least about 90%, 91%, 92%, 93%, 94%, 95%, 96%, 97%, 98%, or 99% identical to a contiguous portion between 15-5522 bp in length of a sequence selected from SEQ ID NO: 9-19.

92. The AAV vector of any one of claims 84-91, wherein the cell-specific promoter sequence comprises a nucleic acid promoter sequence identical to a contiguous portion between 15-5522 bp in length of a sequence selected from SEQ ID NO: 9-19.

93. The AAV vector of any one of claims 84-92, wherein the cell-specific promoter sequence comprises a nucleic acid promoter sequence at least about 90%, 91%, 92%, 93%, 94%, 95%, 96%, 97%, 98%, or 99% identical to a sequence selected from SEQ ID NO: 9-19.

94. The AAV vector of any one of claims 84-93, wherein the cell-specific promoter sequence comprises a nucleic acid promoter sequence identical to a sequence selected from SEQ ID NO: 9-19.

95. The AAV vector of any one of claims 84-94, wherein the cell-specific promoter sequence comprises the nucleic acid promoter sequence of SEQ ID NO: 18.

96. The AAV vector of any one of claims 84-95, wherein the cell-specific promoter sequence comprises the nucleic acid promoter sequence of SEQ ID NO: 19.

97. The AAV vector of any one of claims 76-96, wherein the nucleic acid sequence encoding the RGS10 protein or functional fragment thereof comprises one of SEQ ID NO: 1-5.

98. The AAV vector of any one of claims 76-97, wherein the nucleic acid sequence encoding the RGS10 protein or functional fragment thereof comprises a sequence that is at least 80%, 85%, 90%, 95% or 99% identical to SEQ ID NO: 1.

99. The AAV vector of any one of claims 76-98, wherein the nucleic acid sequence encoding the RGS10 protein or functional fragment thereof comprises a sequence encoding an amino acid sequence selected from SEQ ID NO: 6-8, or a functional fragment thereof.

100. The AAV vector of any one of claims 76-99, wherein the nucleic acid sequence encoding the RGS10 protein or functional fragment thereof comprises a sequence having at least 80%, 81%, 82%, 83%, 84%, 85%, 86%, 87%, 88%, 89%, 90%, 91%, 92, 93%, 94%, 95%, 96%, 97%, 98%, or 99% identity to a sequence encoding an amino acid sequence selected from SEQ ID NO: 6-8, or a functional fragment thereof.

101. The AAV vector of any one of claims 76-100, wherein the AAV vector is formulated for direct administration to a central nervous system (CNS) of a subject.

102. The AAV vector of any one of claims 76-101, wherein the AAV vector is formulated for direct administration to the microglia of a subject.

103. A kit comprising: i) a composition comprising the AAV vector of any one of claims 74-100, and ii) instructions for use.

104. The kit of claim 103, further comprising a device for delivery of the AAV vector.

105. The kit of claim 104, wherein the device comprises a catheter or other device for intrathecal administration of the composition.

106. The kit of claim 104, wherein the device comprises a catheter or other device for intravascular administration of the composition.


SEQUENCE LISTING (55 sequences)

SEQ ID NO: 1 - DNA, 522 bp, synthetic construct
  source 1..522: mol_type = other DNA
atgcagtctgaactttgctttgcagacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgcttcttaaagtctgacttgtttttaaaacacaagcgaaccgaggaagaggaagaagatttgcctgatgctcaaactgcagctaaaagagcttccagaatttataacacatga

SEQ ID NO: 2 - DNA, 748 bp, synthetic construct
  source 1..748: mol_type = other DNA
tttcttcctcctcgggcgcccgcggcgatgttcaaccgcgccgtgagccggctgagcaggaagcggccgccgtcagacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgcttcttaaagtctgacttgtttttaaaacacaagcgaaccgaggaagaggaagaagatttgcctgatgctcaaactgcagctaaaagagcttccagaatttataacacatgagcccccaaaaagccgggactggcagctttaagaagcaaaggaatttcctctcaggaccgtgccgggtttatcattgctttgttatttgtaaggactgaaatgtacaaaacccttcaatgggatgtgtgttttattaactgcttcaccagtaaattttgcatgatggctaagctaa

SEQ ID NO: 3 - DNA, 664 bp, synthetic construct
  source 1..664: mol_type = other DNA
ggattgttggtctgcgtggaacttctcaggtggacaccagagcatggaacacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgctttcttaagtctgacttgtttttaaaacacaagcgaaccgaggaagaggaagaagatttgcctgatgctcaaactgcagctaaaagagcttccagaatttataacacatgagcccccaaaaagccgggactggcagctttaagaagcaaaggaatttcctctcaggacgtgccgggtttatcattgctttgttatttgtaaggactgaaatgtacaaaacccttcaat

SEQ ID NO: 4 - DNA, 573 bp, synthetic construct
  source 1..573: mol_type = other DNA
tttcttcctcctcgggcgcccgcggcgatgttcaaccgcgccgtgagccggctgagcaggaagcggccgccgtcagacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgcttcttaaagtctgacttgtttttaaaacacaagcgaaccgaggaagaggaagaagatttgcctgatgctcaaactgcagctaaaagagcttccagaatttataacacatga

SEQ ID NO: 5 - DNA, 547 bp, synthetic construct
  source 1..547: mol_type = other DNA
ggattgttggtctgcgtggaacttctcaggtggacaccagagcatggaacacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgctttcttaagtctgacttgtttttaaaacacaagcgaaccgaggaagaggaagaagatttgcctgatgctcaaactgcagctaaaagagcttccagaatttataacacatga

SEQ ID NO: 6 - protein, 173 aa, Homo sapiens
MQSELCFADIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 7 - protein, 181 aa, Homo sapiens
MFNRAVSRLSRKRPPSDIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 8 - protein, 167 aa, Homo sapiens
MEHIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 9 - DNA, 1289 bp, Mus musculus
  source 1..1289: mol_type = genomic DNA
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 10 - DNA, 821 bp, Homo sapiens
  source 1..821: mol_type = genomic DNA
aaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatgaggctggctgtgcttttctcgggggccctgctggggctactggcaggtaaggaggaaggaggctgaggggagggggcccctgggagggagcctgccctgggttgctaaccatctcctctctgccaaaagcccaggggac

SEQ ID NO: 11 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 12 - DNA, 315 bp, Homo sapiens
  source 1..315: mol_type = genomic DNA
caatgaaagaaggtgttgagagatgggagatagatgttgatgggaaagaaaaagggagagaccatgggaaggagagagagagagaatgatatacattaggcagattaacttctctttattgtgaagaaagggggaaattaggattttctaggctgtcttttagttgcagcaagtcagcaaaaagggctatgatgaccaggcagcattgagcaactctgactccgccttcccctcttcgcacaccttctggttactgaaaacccagcgttagtagaaaagtttcttttctttgaatgacagaactacagcataatg

SEQ ID NO: 13 - DNA, 2025 bp, Homo sapiens
  source 1..2025: mol_type = genomic DNA
gcaacctccaccttccggtttcaagcgattctcctgcctcagcctcccaagtagttgggattacaggcacccgccaccacgcctggctaatttttatatttttagtaaagacagggtttcaccattttggccaggctggtcttgaacccctgatctcgtgatccacccacgttggcctcccaaagtgctggaattacaggcgtgagccaccatgcctggcccacattacattcttactcacctccccctaccatggaattttattccacagatatgctattggtttagctactatatgtatatctgtgttttatacataaagcacaagaaccttccagaaccaattttcgccaccttggaagtaataccacctctactaagaatgcacagcatagaccataaaacctcaatgctaagttcaaatattggccctaccacacatgagctgtgtggtcttgtacaagttacataacttctcctccttgtctcaaactcctcacatataagatgaggataataatagtacctgcggccacacacagtggcttaaacgtgtaatcccagcactttgggaggctgcggcagaaggatcactcaaactcaggagttcaagagcagcctgggtacatggcgaaactctgtctctacaaaaaatacaaaaattagctgggtgtggtgatgtgtgcctgtagttccagctacttgggaggctgaggtgagaggatcgttcgagcccaggagatcaaggatgcagtgagctatgatcatgtggctgcactccagcctggataacagagccagaccctgtctgaaagaaacaaaaacaaaaacattagcacctgcatcatagggtcactgggggcactacatgagttcatgtacatcgaggacttaggacattgcctcaggcagacctagtgctgcacaattgcttatgtaattattcccaaatttctccagggcccacagaagaacatggaagtatcttggtttggcaattaaggtgaatcacattctcactctccttttctgcatctctaccccacattcccacaaagctttattcacaccaagtctccagtccttgcctgcattgtgtgatgggtgcctgcagtgatgggtggggacacccatcactgtccagggcgtccccaccatcctcacagcctctctgtctggcctcctgcctttgagccagcccaccacactctcatttctctgcccagcagaaaccaaactgtcctctgcatttactgtctcaactggaagagaaatgcagaatgacaaagaacttgtgaacaagggtcagctccaacagagagtgaagccaaaggggctgggcagaaagagagatgaagacgggggtctgaggaataaggctgtaccagagtgagagtacgggggaggggttgaacaagagttcagggaggagagaattcccagcgctgagccagagactcctttacagaggcccaaggaggcgtggagggagggggaaggctgccaaggctctttctgtctccatgagtgtgtcaagaatgcaaagcactaatgctcttcacttggtccatcttgcagggttgagtttgcagtgagcaaccttgaaggatgagctgacatctcgctcagggccaaataaccgacttgcttactgcttgctataaaatggcacgttacccaaggtcagagttcccttcctataacctccccatccctcacacattcacaggtatctatccaagccatggcatcactctgtggggcttgggggcaaggcaactgacactgcacgctggttctcatgcttgccaagcatgaagccctgtgctgctagcagctgtggaacatagccgttagctttaaaagagggtaaaatcacgtcctggacaggacagccaggtgagttgggaagggaagagagcctgccacgggcacaggcatgttgggggaagtggaagtggtgagagcacagtaggaagtgagaaggggcgggccgtgcttaccaggccgtggacttaaaccagg

SEQ ID NO: 14 - DNA, 5522 bp, Homo sapiens
  source 1..5522: mol_type = genomic DNA
ccaagtcctcagcttaattcatatgggaaagaaaaaaaaagagatgtaggatcttagcccagcagagttagagaggtcccaagaatctcctggtgtgcccgctcccaccaatggacatctggtctgcagcacagtgggatggggaggtgccatgcctcagtttccccacctataaaacagggacaatcatcgtacccactcattggtatgttaggcattttaaatgcatgggaagactttagaacatcacctgtcaccaaataaatggctcagtaagagttagtcactatgtttattacctagtctacacccagcagctcaccccggcacccccacacatctgtgtttactgggaagccaatctcttttaaggaaaattttcttgaaggagaaaagagaacttgaaacccctatcaggctgtccgggaagaaggcccgttgcctctgcagcgtcagcatttttccagggcctaaaataggggcaaaggagggaagtgagagctgattcaagcctccgaggggctgccaggcgccccccagcctgtgtgggcggaaacacattgttggcgtcaggctcagcccccggtttccgctcagcctgtggcccttctacagggatgttctcacttctgcttccccagcctcctagccactctcctctctccaggcagggagagggagtgccacttgcggggcagctgtgaggctgggctctgacgctgacccactggctgaccctggccatccaactcctcagctctggcccttcctcttcccagtgatgggggcacaatgacaggccagccccgagggctttcggtgaattcagggtatctccattgttctctgcaaacccccttgaaatggaaattccccctgccccaccatagagcccagaattggaaaggtttcctctctcaagtcctttcaggatggcttaattcacgttttgattttgtattcatcagaatgtgcatcccttctccccaagactctgatcagcaaacgagattgagtgaattgtgaatactgccaagcctgccacctggggttgtagccctcctatgcagtgcaactgccaggaagagggacaagtgtcatttctagcagcctttttagcaaatctcaacagagctggggctgccctatcactgctgcagcaatctagggcccccaggccggggttttatagcaggaaacagcttggagtgtgggctcacaccaacttgggtttggatcttggttcttcccttaccagctgggttagtttcttaacctctttgagcctcaggctattcttctgtctgatgggaataatcccagctcccaccccaaaaacttcagctgaaggttgagtgagatgatgtatagaactactcctgccctgggcacctgctttaattgctacatttctaatgtcccttctgggccaactctgatcgctcaagattttaaccttggattctgtgattccatttgttccttttttacacctgcctcacaccttacagtgggcttctcccatcccttcccccagcctgtctctctagaccctgccttggcctccaggaaatgtggtcagggagcggtgtctgccctattttgtcctatcctattgtcccaggtattcacgcaggataaggctgatacccgcaacttccgcagccaacttcccctgctggcttggctagcagggaaggaagccggcttggcccccatcctcctcggggtgggggcatgggcttatgaacattaactgaggtccccttctgtgcctgtccattcttcccccctctcatctccactccccaattctccccatgccccacacctctccactagctcccacttgcctggaagcccctccctcatcacttccagaatgtgccccaatctctactcttctctcccaacacacacacacacacacacacacacacacacacacacacacacacacacactgcacactaaggctggacacagcccctcttcagcccaggatctcatcgcagtaatgaagagagagcccccctctctctctagggtcctccaataccatccccagtctcctcacagtggcccctcatccactcatcattggtctgttgggctgaaaacttaggtttccagcctccccatctcttctttctcttgggactggaccctggaaaaccatctggcccccctcccacacaccatgcccctgtgaagggaaaataaaaacttgggaccctaattcactctgccaaaaggaaaaatcaagctggaaactgagtcatgcaagaagctgccttgccttttattcctaagcagatagcgacagataaaaggtaaaatatcccacaggcagctcctctatgttcactttccctatgtaaagagccaatttactgaaccgagataattgactgttcccctacctgctgcttttcttttgcaacatgtggattaccacaacctccccttttcccctccagcccactttcccctttaaatattaaagccctcgaattcatccagaatggcacagaccacagaatgtttttgtgactccatgtttttttctcgccagcatgtccttaaccttggcaaaataaacttctaaattgattgaagccaggcacagtggctcatgcctgtaatcccagcactttgggacgccaaggcagggggatcgcttgagttcaggagttcgagaccagcatggccagcatgataaaaccctgtctctaccaaaaataaaaaaattagctgggtgtggtggcaggcaccagtaatcacagctactcaggagtggcagaattgcttgaaccccggaggcggaggttgcagtgagccgagatccatgccattgcactccagcctgggcaacagagcaagactcggtctcaaaaagatagaaaaaagaaagaaagaaagaaagaaagaaagaaagaaagaaagaaagaaagaaggaaggaaggaaggaaggaaggaaggaaggaaggaaggaaggaaggaaggaaggaaggaaggaagaaagaaaggaaggagatggagtcttgctctgtcgcctaggctggagtgcagtggcatgatctcagctcactgcaacctctgcctcccgggttcaagtgattctcccacctcagcctcctgagtagctggaactacaggaacccaccaccacacccggctaatttttgtattttttagtagagagggggcttcaccatattggcgaggctggtctgaaactcctgatcttgtgatccacccgcctcggcctcccagagtgctgggattacaggtgtgagccaccatgcctggccacaagactccatctcaataaaaaaaataaaattaaattgagacctggctcagatacttttccgtttttgccccacaggcaggttgtgggtgtaaggataggacaaggcagtgaaatcaaacctgtgggactctctgagatgaagggggtggcgggtacactgtgtgtgcatttgtcagacctcattgaacttaagaacctgtgctttctactgcatgtaagttacattgcaaaataagaagtccaggccgggcgccgtgactcacaccagtaatcccaacactttgggaggccaaggcaggtggatcacctgaggtcaggagttcgagaccagcttggccaacatggtgaaatcccatctctactaaaaatacaaaaattagccagtcatgatggtgtatgcctgtaatcccagcttgggaggctgagacaggagaaacacttgaacccgggaggtggaggttgcagtgagccgagattgtgccactgtactccagcctggtgacagagagagactctgtctcaggaaaaaaaaaaaaaaaacccaaatactcaagctcagtgtgattgcctgggccagagaaggatggtcagtgggccatctcctcctcatcagggaccagaagcgccacggccttagggtccttctgtgaagaggccagtatgaggctgtcctttctccccttcaatccccatcctcccccaaggtccctttgaataagcaggcaatagaaatgtgtttgttgggccaacaagccaagcacagcagcccctcacctctagtcatgttccctcccagatggaaattctgcagaactgctacagcctggggacctcctgagacttggcagcctgtgtctgggagggctctggtccaggagaccctcactggcctcttcccaatccctcctctagccctatacctatttcaccccgaattcccctctcctgagccctgggccagagaggccaacctcttgtctctcccctctgaccccttacacaagccacccaaggaagaaccaccacccctatcaggcccttccaaatcagatcttgcatagaatcatcgaatcacaggctggcagagctgccagagccatcaggagctctccaggtcctcccaactcagtgtaggtctggaccagcagtgccggcatctcacatctcccaggagtgtgttggatatacagaatctcaggtcgcacctgagaccgactgagtctgaatctgcattgtaacaaaatcctcaggtaacttgtatgcacaggaaagtatgaaaagcactgctctggtctagtctatcactgtccccctgcccccaacacctgcaattgaatagaagtggacactgaagcctcaagagtatcagaggcttgctcaggacacacagtgagtcgctggcagagctgggacagatccacagcaccaggtggaccagttccaccacacccatctcttttcaccccactacctctccaaagcagatacccacaagcctgcagtccagtgttggggagactaaaacagcctgaggacacccagtgaggggcaaggactgaatcctttcacaggggtcttctccatcacaccccaacaaagtcccccaccccccgttctgctcttaccatgccaagctgtggccaccagcaggagcagcagaactcctgggcccatggcctcggtggggaagtggcaggcaggtgcagggctgcaaggtgcccagggcacagagctctcagctactagctccgcagggatcgggacactggacacacgttcctctcctctgcactggctgtttgtcttgttttcctcttcctcctccttgggctgatcctcttcttccccttttagctaggcaaggcaaccacagagtttggaaatcttgggtctttaagaagaaaacaaagccttgaacccaagagggaggggtcgcagtctggggcagggccagcctgagcttggggggagctagctaagtttggggcccttctaaagccatcttcaaggggtcttgggagaagagacatgtccaagagcagccagaccgttagtcagtagcacaaatgagggtcttcagagtgatcaggtggctctggcagatcccaaagagaagttaggttgcatgatattgtgacatatatattttttctttaccctgatgtcctggcttacaactcctaaaatccttggaatctccaaagta

SEQ ID NO: 15 - DNA, 1402 bp, Homo sapiens
  source 1..1402: mol_type = genomic DNA
ttgttgcttgtgtttcctgttccagcaatgtgttaagcaacatctgaaagccagaagacagttatccctcacaccagtttcctaatcatcactaagccctggggaaacacaatggatcagcgaggccccaaatgcagatgtgattagcacactggtcagtcagcccgcaaaacactttcaccaagtcctctaaagtgttgcataaggcaaagataacgttaagtctgtggaggcttccgttatcgggaaaagatgctgtagtgatcttttctgagtgtctcctacttgcgacaaggtggacttgggaggaaagccgtctgccaaagcctgaagcctccaaggtatagattccctctccatagacattcccctttcttttatatctttcgttaaaatgcctcccagtggactactggggatgtctgcaaaggtctctgtttggttgctatgtagaggtttggaacatttaactgtgaacatggcttctctttaggatgtgatgattcctttggagccatgataaaaagtgttagacataataatacagcaatttctttgggaaaatgtgggtatggatacacacacatgcacacgcacctcttaggtacattaaatcccactggctgcacctgagtttgtctgccagctctgatgatgaggggtggtctcgatatgcctgaagtatggggtctgcaaagtgcattttagcatagctcaggggacagtggagtgtaatgatagcttgaggttctgatttttagcttctcaaattgctggctgtgtgggcttgggaaagctacttaacttctttgttcctcagcttcttcatctgttagatggaaaggatcatagcagtagtacccattcatctgatgttgtgagaactaaattagtaaacatggataaagcccttagaacaaatgcctgccacatactaagagctggagagatgaacttaactggcagtttcctagaaaatctacatttctctgattttgtgtgtgtgtgtgtgtgtgtgagagagagacagagtctcgctctgtcacccaggctggagtgcaatggcacgatttcggctcactgcaaactccgcctcatgggttcaaacaattctcctgcctcagcctcctgagtagctgggattacaggcgcgtgctgtcacaaccagctaattttatgtgtttttagtagaaacagggtttcaccatgttagcaaggatgatctcaatctcctgaccttgagatcctcccgcctctgcctcccaaagtgctgggattacaggcatgagccactgcacccggaccatttctctgatttttttaaaaataaataactgatttaaccaaattttagaaaacatagtttgtttcacctggaggctgaaaattctccctgattatt

SEQ ID NO: 16 - DNA, 3002 bp, Homo sapiens
  source 1..3002: mol_type = genomic DNA
cacatgcacacaccacacatatatgcatacactccacatacacacaaccatacacatacaccacacacactcacctacacaaccacacacacatgcacacaccacacactctgagccaggaatggcttgttcccgctggtggacatcacctgtcagatgctaaatgaggaaaggaccccctcacctctctggcacctgttagcaaaggttcctacagacaaccttgagctttggggtctgtgacctgccagtcacctccctgcctcccacccccagtcaagcctaggagagagcttttgtggcaacaactaccacttctcactgcagagggcagctgggaccatgggtggctggagacccactcaagttctggctgggtcaagaatcccggctccatcgcatccagctctgtggtctggctcctgtgggcctcactttccccatccgtaagttgggacagggtgaccacaggtagcacctctcagagctgttagggccaaatgagacaactttcagagagtgcctggtgcgaagtagctgagccccgaggaaagatagggtctggggtagctgaattccttccctagccgaactccaggttcagccagcaatttcccaccctgagtcgacaaatgagcaccatcgccggatgggaacatgcgctcactgtctggtccggccgccacccagtgaaagaccaaccttttcaccccgttcagtgaaacctctttagctctggccgccaaggaatgcagaaccaaacacacacatgcacacacatacacatacacacacatgcacacatacacctacacacacatatacatacaacacacacacacacacacacacgaagtcagaggcagagggcaagaaaggagaacacacaaactgccactggctctttaaggctgagaccctgactctcagagtcccctgcaccccacaacccctcctggcttccagggaaaggtgggggcaacgtatggaggacccagccccacctgcagtcagcagagcctcaaaaaagatggttgtgtggccctggacggcccctgccctctctgggcctcagtttcccatctgtggaatgaagcactgttctgggcagctctgaagtcattattctgcctacctccccccggaccctaattttttcaaggactcagaatctaacaggccattaggagctggggtcaggcccaacgcctcttgcttcaccttagaggtaactcaggtccagagactggaaacagcctgcccaggtcacacagaaagtcaggggtgagcccagcagagccctggacagagaagcctgtcttggtatatcccagaggaggtcacttcccggctatgtgtccttgggctgggcatgtcacctctctgagcctcggtttccatatctgtacaatggggataagagccacctccctggactgctgttaggacaagatgcggccatgcctggtgcacagtgggtgccaattagcaggtaccacgtgattgcccaggactccctgcctggggacatgtcaggcagggctggccgtgtgtaagtgtggcaaatgccctaagatggtcctgagtctgtggaggagtgcagatgcacccctgaggcctgggggccccaagacttgaagctaggggaccgggggctgccccacccccacccagagactggtggcctttgggggaggggcggcccacggaaacctggcttcaatcagtggccttgtgggggaggggtgttctggttggagcactgcacatgcccccgaccccagggccagccaaggacagggacctgggccctggaggtctggatggggaggggggtcctgggggcacaatccccctccacacacacacacacacacagagcagcatggacacacaatataaagacacacaacacaggcacaccgactcacacagcactgagacacccaacagcactctcatggcccatgcacacaatacacagagaacacacacggacacacaacacatatacatacacaaccccctgacacacagccgatccctgcacacaggacaataccgtccacatcaccctcacacagatcctggaagcaacagcctggcatccacttgcagaaaagtggaagcaacacaccccctacaccaaggccccagccgcacacacacaacactccggcccaccctggcccaccccagcaccgcacacgggactcagattccaccacactcatgcacacaacaccaaacacacactcaaagcccacacacacttagagaggccccccagagtgctccaacagtccccagtccctaagctccaagatagctccaggctctcaggacagaggaggggaccccaggactcaccagttccttggcgtacaggactgggtgctcccgccgaatggagcgagagtggggggcacctgggctggaagagagagtccgtgctggtgcccggggctgctctgggctggcgcctgcccgccctccctcctcccgtccctcctcccgtccctcctccttcctccctccccggctgggccgcagggccagcttgatttcattaagagggtttggggtggggacagggggaggggaggggctactctcgggccacacgcaatcccacccttgctcacactctcgctctggggaagaccagatgcggcacttcctccctgacctcgggtccccagggcaggctgagacagacagacaaggctgtcctgcagcgctcccccagacagtgagctggggttctgcccacgctgctgcccaataccctccaaaggggacaagctcagggaggttcagaggcctctggaagtgggaaggaaaattccccgtccttgtgtggctcaggcttccaggctcagggtgcagcgtgggagctgggattcgttttagagacaggcctgatggtgggtccgagcctgagacagggagcccaactctgcccttccctctccagacctcagtttcccatttgtagggat

SEQ ID NO: 17 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 18 - DNA, 1760 bp, Homo sapiens
  source 1..1760: mol_type = genomic DNA
atatctgttgagtgaacgtatgaataaagaagtgagttcctcccagcaggcactgagaacattgggagtacagggttgcagctctctctgcagcaggagaatgtagctgcaataaagggaagtcaagaagccagagtccagccaggtgcagtggctcatgcctgtaatcccagcactttgggaggctgaggtgggtggatcacaaggtcaagagatagagaccatcctggccaacatggcgaaaccccatctgtactaaaaatacaaaaattagctgggcgtggtggtgggcgcctgtagtcccagctactcaggaggctgaggtaggagaattgcttgaacccaggaggcagtggttgcagtgagccgagattgcaccattgcactcccgcctgggcgacagagcaagactccgactcaaaaaaaaaaaaaaaagcagcagcagcagccagaggccactccagcatctcccctacctggcttgggtcagggagagggcagtgagaagtgaaaactcccagctacagaaaaggaaatatgttggggggaagggagaaggaaaggtgtcttcatcaatgccggggcagggtagatggagccctgggcagggagtttggaccaggaaatctcaatgagggaaatgtgctgtcctcacctctccaagaagcgactggccaaacagagtgacagaggggataaaggttatgcctagggaggcatgtgtcagaggctatcatccactctgttgaacccacagtgaccagcaccaccatcacacaaacatgcctgcatgtgtgcacgcacgtgcagtgtgcaaacctgatgtcagcctcactccctggctcttctgtccacaaacgctgtttctttaagtaccactttcagttcctccaaagaatctacttaaactcttaaattcctgatctctatagattttactaaagatttcaaaggagataagatgagagggttacgttgcacattctaaagcaaacaaattaaaatgttttgttagacatttccatatttttaagggcctccttggagctgccaggctgggagtgaggtttctctccctttctaaaccctgtgcccatcttgtcaccctcctggagctgccagcagacttcagattcttctccgatctacagagcagaaaaattcagccagcccttccttgtcttcctatccacagctgcctgcccagactcatgaaacctgacaaaatgcaaggtcttatcattacctgaaccttggacctgttcaaaaatactagttcctgagaataaatatccctggtgtcttcctgcccttcctgcacacctccagtggcttatcaaaatatttgtttcatgcgcacactgggctctcatttaagaggaatttgggagaatgttattttctaatctgcatttcacaccaggctccccctccttcctggggtgctagtgtcagcagaacctgatggggaagtgaggtctgggaggcagaggaggaaggaatgaggggaaaggggaagtttgggaggaaggcttctgagaagactggtgggagagaaggagagcctgcagacagaggcctccagcttggtctgtctccccacctctaccagcatctgctgagctatgagccaaaccagggatttacagggtagggagggtgggataggcagcggcattagatcggaggaatgagatggacagacctgggctgtgggctaggagggcagtcagctggcctagggtagcccgggctggtgt

SEQ ID NO: 19 - DNA, 1678 bp, Mus musculus
  source 1..1678: mol_type = genomic DNA
tactataggatgcatcgtgaaaacctcgtttccaccaggaactgaggttgctgctggaggaaatctcccatgaagagctgttaactggggaactggttggatccagctcctacaagctctcctttaatttccatacacatgccaagctgtgaacacctgcacacacagtacaaataaaacagtagaaatgaaatgaaaattaaaacaaaaacagcatactttctcctttctacccacatatctcctgcatccctgagagcaatttcttggccatcttccataatcatgctattcaaacctgccctctaagtacagagctctgaatggagaccacgggagcagacttttcatctgttggctccctgggcctagcagtgtaccctgcaaacaggaaaccttcgctcatcatttatcacatgaatgtggctagagaactgtccccaccagacactgagagcctctcacagggccagagggtggctgcctcaaaggcaggcgagaggctgcagtcttctgcagagtggacagatactgcctgccatacaaggggacatagcctggggcggggggcacaagggaaggggcctgagtgcattggagtcacagccctccctcccctggtgacccagtgactgaggggaccaaggctatccctggtatgaggggagggcatttgcatgagttagacccctgacagcccatggtggggacaggaagtagctctgtctactgtctctttcagtctcactttctgttcctccaaagtgtctcttcaaactctcaaatgactttgtagattcagcctaagatctcaaagcaggtgaggcaggaggctggcactcaagagcctgacgaacacaaacccaagtccttctgggacatgtctccatgtgtccccagtgctcctctgtagcccctgagagaggtttctttcctttccagggcgctaggctcagctcaccccattcctggagcagcctgcagacttcatcctctctcttccatcccggggaaagtcagccagtcctcctcagctgcctgtcttaacctgcatcatgaagcctgaggagatttcaagtaaaccctcccaagccccacctcaggatctggggaaagccactgtctaccgcatccttggtttgagacaggttcttactgtgttggcctggctgggttccaactcccaggatcctctagcctcagcctctcttaggtgctagattgcaggcctagggtatcacgctcggtccctcagatcctggtgcctttccacacctccgggagctgatctaagtctttctcccacgtacagtctgcttctttggaggagctgtgtggagctaggtgtgttacagggctgtagctccagccatctgggaggctgaggcaggatggctacttcaattctcaagagcctggataggagaccctgcccccaccccacccccaaagtaatttttcagcacattacttcttcatctcctctctcaacccgttctgccttctcctggggtgctggtgtcagcagaagctgatgtggaagtgatgcctgggagttagcaagggaatgagtggaaaggggaagtgtgagaacggtcccagaagagactggggagctggtggagagaggacccagcggacagactgccagcctaagacaaccagcgtctgaggagcc

SEQ ID NO: 20 - DNA, 21 bp, synthetic construct
  source 1..21: mol_type = other DNA
cggccaagtccaaagacatac

SEQ ID NO: 21 - DNA, 21 bp, synthetic construct
  source 1..21: mol_type = other DNA
tgtctggttggcaaggagttt

SEQ ID NO: 22 - DNA, 21 bp, synthetic construct
  source 1..21: mol_type = other DNA
atcaagccttctcctgaggaa

SEQ ID NO: 23 - DNA, 19 bp, synthetic construct
  source 1..19: mol_type = other DNA
gccagcagttcatcaatgc

SEQ ID NO: 24 - DNA, 20 bp, synthetic construct
  source 1..20: mol_type = other DNA
gggctgaatcgttggaaaac

SEQ ID NO: 25 - DNA, 22 bp, synthetic construct
  source 1..22: mol_type = other DNA
attccgacttcaggaaagcttt

SEQ ID NO: 26 - DNA, 18 bp, synthetic construct
  source 1..18: mol_type = other DNA
gcggagaaggcaaagcaa

SEQ ID NO: 27 - DNA, 24 bp, synthetic construct
  source 1..24: mol_type = other DNA
gtggtcaatgttcacctctttagg

SEQ ID NO: 28 - DNA, 21 bp, synthetic construct
  source 1..21: mol_type = other DNA
gggagccagaaaacacagact

SEQ ID NO: 29 - DNA, 21 bp, synthetic construct
  source 1..21: mol_type = other DNA
ttgccttgttttgcattgttc

SEQ ID NO: 30 - DNA, 22 bp, synthetic construct
  source 1..22: mol_type = other DNA
gagcaagcatctctgaccatct

SEQ ID NO: 31 - DNA, 22 bp, synthetic construct
  source 1..22: mol_type = other DNA
actgtcgttgactggtcctcat

SEQ ID NO: 32 - DNA, 16 bp, synthetic construct
  source 1..16: mol_type = other DNA
cacccagcgcctttgg

SEQ ID NO: 33 - DNA, 21 bp, synthetic construct
  source 1..21: mol_type = other DNA
gacaataaggttgcccaggtt

SEQ ID NO: 34 - DNA, 22 bp, synthetic construct
  source 1..22: mol_type = other DNA
ttggctagcgtgtgaagatttc

SEQ ID NO: 35 - DNA, 19 bp, synthetic construct
  source 1..19: mol_type = other DNA
tggccttttcctgcatctg

SEQ ID NO: 36 - DNA, 23 bp, synthetic construct
  source 1..23: mol_type = other DNA
tgctgagctagactatgccatct

SEQ ID NO: 37 - DNA, 21 bp, synthetic construct
  source 1..21: mol_type = other DNA
ctaaagccccttgtttttgga

SEQ ID NO: 38 - DNA, 21 bp, synthetic construct
  source 1..21: mol_type = other DNA
gctggatctcgttccgattaa

SEQ ID NO: 39 - DNA, 19 bp, synthetic construct
  source 1..19: mol_type = other DNA
ccgcaacacttccgtgact

SEQ ID NO: 40 - DNA, 25 bp, synthetic construct
  source 1..25: mol_type = other DNA
attatacagacagtggcgtgttttt

SEQ ID NO: 41 - DNA, 23 bp, synthetic construct
  source 1..23: mol_type = other DNA
aaattgcacgacacaccaagtac

SEQ ID NO: 42 - DNA, 17 bp, synthetic construct
  source 1..17: mol_type = other DNA
gccccggccagatatgt

SEQ ID NO: 43 - DNA, 20 bp, synthetic construct
  source 1..20: mol_type = other DNA
gcgcatagctgtcgaacttc

SEQ ID NO: 44 - DNA, 23 bp, synthetic construct
  source 1..23: mol_type = other DNA
cctggtacttgctactcgctttt

SEQ ID NO: 45 - DNA, 19 bp, synthetic construct
  source 1..19: mol_type = other DNA
agcacgtcgtggagaggat

SEQ ID NO: 46 - DNA, 21 bp, synthetic construct
  source 1..21: mol_type = other DNA
ggagagcatccaggtcctaga

SEQ ID NO: 47 - DNA, 23 bp, synthetic construct
  source 1..23: mol_type = other DNA
tgagaccaggacaagacttcatc

SEQ ID NO: 48 - DNA, 23 bp, synthetic construct
  source 1..23: mol_type = other DNA
cttctcctacagagccctgactt

SEQ ID NO: 49 - DNA, 20 bp, synthetic construct
  source 1..20: mol_type = other DNA
ggccaagagggcagatctac

SEQ ID NO: 50 - DNA, 17 bp, synthetic construct
  source 1..17: mol_type = other DNA
gcaaccccctccactca

SEQ ID NO: 51 - DNA, 21 bp, synthetic construct
  source 1..21: mol_type = other DNA
tgtgctgtttctcagcctcat

SEQ ID NO: 52 - DNA, 19 bp, synthetic construct
  source 1..19: mol_type = other DNA
gcctgctgcttctgttggt

SEQ ID NO: 53 - DNA, 24 bp, synthetic construct
  source 1..24: mol_type = other DNA
ggtcttcttggtttctgccagtga

SEQ ID NO: 54 - DNA, 23 bp, synthetic construct
  source 1..23: mol_type = other DNA
caaggtcatccatgacaactttg

SEQ ID NO: 55 - DNA, 20 bp, synthetic construct
  source 1..20: mol_type = other DNA
ggccatccacagtcttctgg